(19) United States
(12) Patent Application Publication
(10) Pub. No.: US 2013/0095097 A1
(43) Pub. Date: Apr. 18, 2013

(54) Polypeptide Heterodimers and Uses Thereof

(71) Applicants: John W. Blankenship, Seattle, WA (US); Philip Tan, Edmonds, WA (US)

(72) Inventors: John W. Blankenship, Seattle, WA (US); Philip Tan, Edmonds, WA (US)

(73) Assignee: Emergent Product Development Seattle, LLC, Seattle, WA (US)

(21) Appl. No.: 13/519,740

(22) PCT Filed: Dec. 29, 2010
(86) PCT No.: PCT/US2010/062404

§ 371 Date: Jun. 28, 2012

Related U.S. Application Data
(60) Provisional application No. 61/290,840, filed on Dec. 29, 2009.
(60) Provisional application No. 61/365,266, filed on Jul. 16, 2010.
(60) Provisional application No. 61/366,743, filed on Jul. 22, 2010.

(51) Int. Cl. C07K 16/46

(52) U.S. Cl. CPC C07K 16/46
USPC 424/133.1; 530/387.3; 435/320.1; 435/328; 435/69.6

(57) ABSTRACT

The present disclosure provides polypeptide heterodimers formed between two different single chain fusion polypeptides via natural heterodimerization of an immunoglobulin CH1 region and an immunoglobulin light chain constant region (CL). One chain of a heterodimer comprises a binding domain that specifically binds a target (e.g., a receptor). In addition, both chains of a heterodimer further comprise an Fc region portion. The present disclosure also provides nucleic acids, vectors, host cells and methods for making polypeptide heterodimers as well as methods for using such polypeptide heterodimers, such as in reducing T cell activation, inhibiting solid malignancy growth, and treating autoimmune or inflammatory conditions.

### CROSS-REFERENCE(S) TO RELATED APPLICATION(S)

This application claims the benefit under 35 U.S.C. §119(e) of U.S. Provisional Patent Application No. 61/366,743, filed Jul. 22, 2010, which is herein incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 910180_{—}419PC SEQUENCE_LISTING.txt. The text file is 667 KB, was created on Dec. 29, 2010, and is being submitted electronically via EFS-Web, concurrent with the filing of the specification.

### BACKGROUND

1. Technical Field

The present disclosure generally provides polypeptide heterodimers, compositions thereof, and methods for making and using such polypeptide heterodimers. More specifically, the polypeptide heterodimers provided herein are formed, in part, via natural heterodimerization between an immunoglobulin CH1 region and an immunoglobulin light chain constant region (CL). In addition, one single chain polypeptide of the polypeptide heterodimers provided herein comprises a binding domain that specifically binds a target. Furthermore, both single chain polypeptides of the polypeptide heterodimers provided herein each comprise an Fc region portion (e.g., immunoglobulin CH2 and CH3 domains).

2. Description of the Related Art

The process of signal transduction often involves receptor proteins that have extracellular domains, transmembrane domains, and intracellular domains. During ligand binding, receptor molecules often oligomerize or multimerize (also referred to as “cross-link”) to transmit effectively the signal to the intracellular component of the cell. The stimulation or blockade of the interaction between a receptor and a ligand or the subsequent oligomerization or multimerization of receptors has important therapeutic implications for a wide variety of diseases.

Molecules useful in modulating receptor and ligand interactions include antibodies or molecules derived from antibodies. For instance, an antibody or its derivative may function as a receptor antagonist that binds to a cell surface receptor and inactivates it by blocking the binding site of an activating ligand or preventing receptor dimerization or multimerization required for activation.

An example of an antibody derivative functioning as a receptor antagonist is Genmab UNIBODY®. UNIBODY® is a half-molecule of conventional IgG. It consists of one heavy and one light IgG chain only by deleting the core hinge region of human IgG4. UNIBODY® molecules bind only one antigen molecule and preclude cross-linking of antigen molecules. However, UNIBODY® molecules have no cytolytic function, such as the antibody-dependent cell-mediated cytotoxicity (ADCC) and complement dependent-cytotoxicity (CDC), and thus may be ineffective for treating certain diseases.

Another example of an antibody derivative functioning as a receptor antagonist is Genentech's one armed monoclonal antibodies developed using so called “knobs into holes” engineering of antibody CH3 domains. Although such molecules may retain Fc activities, they require at least three polypeptide chains. Coexpression of multiple polypeptide chains in a recombinant cell generally results in a mixture of both homodimers and heterodimers. The costs associated with recovery and purification of heterodimers from the mixture has limited the commercial application of this technology.

Accordingly, there remains a need in the art for alternative polypeptide heterodimers and efficient methods for producing the same.

### BRIEF SUMMARY

The present disclosure provides polypeptide heterodimers formed between two different single chain polypeptides via natural heterodimerization of an immunoglobulin CH1 region and an immunoglobulin light chain constant region (CL). The present disclosure also provides nucleic acids, vectors, host cells and methods for making polypeptide heterodimers as well as methods for using such polypeptide heterodimers, such as in reducing T cell activation, inhibiting solid malignancy growth, and treating autoimmune or inflammatory conditions.

In one aspect, the present disclosure provides a polypeptide heterodimer that comprises (a) a first single chain polypeptide comprising a binding domain that specifically binds a target, a hinge, a first immunoglobulin heterodimerization domain, and an Fc region portion; and (b) a second single chain polypeptide comprising a hinge, a second immunoglobulin heterodimerization domain that is not the same as the first immunoglobulin heterodimerization domain of the first single chain polypeptide, and an Fc region portion; wherein the first and second immunoglobulin heterodimerization domains associate with each other to form a polypeptide heterodimer comprised of the first and the second single chain polypeptides, and (i) the first immunoglobulin heterodimerization domain comprises a first immunoglobulin CH1 region and the second immunoglobulin heterodimerization domain comprises a first immunoglobulin CL region, or (ii) the first immunoglobulin heterodimerization domain comprises a first immunoglobulin CL region and the second immunoglobulin heterodimerization domain comprises a first immunoglobulin CH1 region, and wherein the Fc region portion comprises an immunoglobulin CH2 domain and CH3 domain of IgG1, IgG2, IgG3, IgG4, IgA1, IgA2, IgD, or any combination thereof; an immunoglobulin CH3 domain of IgG1, IgG2, IgG3, IgG4, IgA1, IgA2, IgD, IgE, IgM, or any combination thereof, an immunoglobulin CH3CH4 domain of IgE, IgM, or a combination thereof

In certain embodiments, the binding domain of the polypeptide heterodimer is a single chain Fv (scFv).

In certain embodiments, the binding domain is amino terminal to the Fc region portion. In certain other embodiments, the binding domain is carboxyl terminal to the Fc region portion.

In certain embodiments, the binding domain specifically binds to c-Met, RON, CD3, CEACAM6, EGFR, ErbB3, ErbB4, EphA2, GITR, IGF1R, GHRHR, GHR, FLT1, KDR, FLT4, CD44v6, CD151, TGFBR2, TGFBR1, IL6R, gp130, TNFR1, TNFR2, PD1, PD-L1, PD-L2, BTLA, HVEM, RANK, TNFRSF4, CD40, CD137, TWEAK-R, LTβR, LIFRβ, OSMRβ, TCRα, TCRβ, CD19, CD28, CD80, CD81, CD86, TLR7, TLR9, PTCH1, LRP5, Frizzled-1, or Robo1.

In certain embodiments, the first immunoglobulin heterodimerization domain comprises the first immunoglobulin CH1 region and the second immunoglobulin heterodimerization domain comprises the first immunoglobulin CL region. The first CH1 region may be amino terminal to the Fc region portion of the first single chain polypeptide, and the first CL region may be amino terminal to the Fc region portion of the second single chain polypeptide. Alternatively, the first CH1 region may be carboxyl terminal to the Fc region portion in the first single chain polypeptide, and the first CL region may be carboxyl terminal to the Fc region portion in the second single chain polypeptide.

In certain embodiments in which the first immunoglobulin heterodimerization domain comprises the first immunoglobulin CH1 region and the second immunoglobulin heterodimerization domain comprises the first immunoglobulin CL region, the first single chain polypeptide may further comprise a second CH1 region, the second single chain polypeptide may further comprise a second CL region, and the second CH1 region of the first single chain polypeptide and the second CL region of the second single chain polypeptide associate with each other in the polypeptide heterodimer. For example, in one embodiment, the Fc region portion of the first single chain polypeptide is disposed between the first and second CH1 regions, and the Fc region portion of the second single chain polypeptide is disposed between the first and second CL regions. In another embodiment, both the first and second CH1 regions are amino terminal to the Fc region portion in the first single chain polypeptide, and both the first and second CL regions are amino terminal to the Fc region portion in the second single chain polypeptide. In yet another embodiment, both the first and second CH1 regions are carboxyl terminal to the Fc region portion in the first single chain polypeptide, and both the first and second CL regions are carboxyl terminal to the Fc region portion in the second single chain polypeptide.

In certain other embodiments in which the first immunoglobulin heterodimerization domain comprises the first immunoglobulin CH1 region and the second immunoglobulin heterodimerization domain comprises the first immunoglobulin CL region, the first single chain polypeptide may further comprise a second CL region, the second single chain polypeptide may further comprises a second CH1 region, and the second CL region of the first single chain polypeptide and the second CH1 region of the second single chain polypeptide associate with each other in the polypeptide heterodimer. In one embodiment, in the first single chain polypeptide, the first CH1 region is amino terminal to the Fc region portion, and the second CL region is carboxyl terminal to the Fc region portion; and in the second single chain polypeptide, the first CL region is amino terminal to the Fc region portion, and the second CH1 region is carboxyl terminal to the Fc region portion. In another embodiment, in the first single chain polypeptide, the first CH1 region is carboxyl terminal to the Fc region portion, and the second CL region is amino terminal to the Fc region portion; and in the second single chain polypeptide, the first CL region is carboxyl terminal to the Fc region portion, and the second CH1 region is amino terminal to the Fc region portion. In another embodiment, in the first single chain polypeptide, both the first CH1 region and the second CL regions are amino terminal to the Fc region portion, and the first CH1 region is amino terminal to the second CL region; and in the second single chain polypeptide, both the first CL region and the second CH1 region are amino terminal to the Fc region portion, and the first CL region is amino terminal to the second CH1 region. In another embodiment, in the first single chain polypeptide, both the first CH1 region and the second CL regions are amino terminal to the Fc region portion, and the second CL region is amino terminal to the first CH1 region; and in the second single chain polypeptide, both the first CL region and the second CH1 region are amino terminal to the Fc region portion, and the second CH1 region is amino terminal to the first CL region. In another embodiment, in the first single chain polypeptide, both the first CH1 region and the second CL regions are carboxyl terminal to the Fc region portion, and the first CH1 region is amino terminal to the second CL region; and in the second single chain polypeptide, both the first CL region and the second CH1 region are carboxyl terminal to the Fc region portion, and the first CL region is amino terminal to the second CH1 region. In another embodiment, in the first single chain polypeptide, both the first CH1 region and the second CL regions are carboxyl terminal to the Fc region portion, and the second CL region is amino terminal to the first CH1 region; and in the second single chain polypeptide, both the first CL region and the second CH1 region are carboxyl terminal to the Fc region portion, and the second CH1 region is amino terminal to the first CL region.

In certain embodiments, the first immunoglobulin heterodimerization domain comprises a first immunoglobulin CL region and the second immunoglobulin heterodimerization domain comprises a first immunoglobulin CH1 region. The first CL region may be amino terminal to the Fc region portion of the first single chain polypeptide, and the first CH1 region may be amino terminal to the Fc region portion of the second single chain polypeptide. Alternatively, the first CL region may be carboxyl terminal to the Fc region portion in the first single chain polypeptide, and the first CH1 region may be carboxyl terminal to the Fc region portion in the second single chain polypeptide.

In certain embodiments in which the first immunoglobulin heterodimerization domain comprises a first immunoglobulin CL region and the second immunoglobulin heterodimerization domain comprises a first immunoglobulin CH1 region, the first single chain polypeptide may further comprise a second CL region, the second single chain polypeptide may further comprise a second CH1 region, and the second CL region of the first single chain polypeptide and the second CH1 region of the second single chain polypeptide associate with each other in the polypeptide heterodimer. In one embodiment, the Fc region portion of the first single chain polypeptide is disposed between the first and second CL regions, and wherein the Fc region portion of the second single chain polypeptide is disposed between the first and second CH1 regions. In another embodiment, both the first and second CL regions are amino terminal to the Fc region portion in the first single chain polypeptide, and both the first and second CH1 regions are amino terminal to the Fc region portion in the second single chain polypeptide. In yet another embodiment, both the first and second CL regions are carboxyl terminal to the Fc region portion in the first single chain polypeptide, and both the first and second CH1 regions are carboxyl terminal to the Fc region portion portion in the second single chain polypeptide.

In certain embodiments, the first CL region is a Cκ region. In certain other embodiments, the first CL region is a Cλ region.

In certain embodiments, the second CL region is a Cκregion. In certain other embodiments, the second CL region is a Cλ region.

In certain embodiments, the Cκ region is a wild type human immunoglobulin Cκ region.

In certain embodiments, the Cκ region is an altered human immunoglobulin Cκ region in which one or more amino acids of a wild type human Cκ region are substituted at N29, N30, Q52, V55, T56, T56, S68, or T70. For example, the one or more amino acid substitutions are selected from Ala (A), Arg (R), Trp (W), Tyr (Y), Glu (E), Gln (Q), Lys (K), Asp (D), Met (M), Ser (S), and Phe (F).

In certain embodiments, the Cκ region is an altered human immunoglobulin Ck region with the cysteine residue of a wild type human Cκ region that is involved in forming a disulfide bond with a wild type human immunoglobulin CH1 region is deleted or substituted.

In certain embodiments, the Ck region is selected from polypeptides comprising SEQ ID NOS:141-178 and 202.

In certain embodiments, the Cλ region is a wild type human immunoglobulin Cλ region.

In certain embodiments, the Cλ region is an altered human immunoglobulin Cλ region with the cysteine residue of a wild type human Cλ region that is involved in forming a disulfide bond with a wild type human immunoglobulin CH1 region is deleted or substituted.

In certain embodiments, the Cλ region is a polypeptide comprising SEQ ID NO:140.

In certain embodiments, the first CH1 region or the second CH1 region when present is a wild type human immunoglobulin CH1 region. In certain other embodiments, the first CH1 region or the second CH1 region when present is an altered human immunoglobulin CH1 region with the cysteine of a wild type human immunoglobulin CH1 region that is involved in forming a disulfide bond with a wild type human immunoglobulin CL region is deleted or substituted.

In certain embodiments, the first CH1 region and the second CH1 region when present is a polypeptide comprising SEQ ID NO:114.

In certain embodiments, the CH1 region is an altered human immunoglobulin CH1 region comprising an amino acid substitution by which Val (V) at position 68 is substituted by Lys (K), Arg (R) or His (H), and wherein the Ck region is an altered human immunoglobulin Ck region comprising an amino acid substitution by which Leu (L) at position 29 is substituted by Asp (D) or Glu (E). In certain other embodiments, the CH1 region is an altered human immunoglobulin CH1 region comprising an amino acid substitution by which Val (V) at position 68 is changed to Asp (D) or Glu (E), and wherein the Ck region is an altered human immunoglobulin Ck region comprising an amino acid substitution by which Leu (L) at position 29 is changed to Lys (K), Arg (R) or His (H).

In certain embodiments, the Fc region portion comprises an immunoglobulin CH2 domain, such as an IgG1 CH2 domain or an IgG2, IgG3, IgG4, IgA1, IgA2, or IgD CH2 domain.

In certain embodiments, the Fc region portion comprises an immunoglobulin CH3 domain, such as an IgG1 CH3 domain or an IgG2, IgG3, IgG4, IgA1, IgA2, IgD, IgE or IgM CH3 domain.

In certain embodiments, the Fc region portion comprises an immunoglobulin CH2 domain and an immunoglobulin CH3 domain, such as IgG1, IgG2, IgG3, IgG4, IgA1, IgA2, or IgD CH2 and CH3 domains.

In certain embodiments, the Fc region portion comprises an immunoglobulin CH2 domain and an immunoglobulin CH3 domain, the immunoglobulin CH3 domain is linked to the CH1 domain immediately carboxyl terminal to the immunoglobulin CH3 domain in one single chain polypeptide via a peptide comprising SEQ ID NO:787, 788, 789 or 790, and the immunoglobulin CH3 domain is linked to the CL domain immediately carboxyl terminal to the immunoglobulin CH3 domain in the other single chain polypeptide via a peptide comprising SEQ ID NO:787, 791, 792, or 793.

In certain embodiments, the Fc region portion comprises IgM or IgE CH3 and CH4 domains.

In certain embodiments, the CH2 domain is an altered human IgG1, IgG2, IgG3, or IgG4 CH2 domain that comprises an amino acid substitution at position 297 and at least one additional substitution or deletion at positions 234 to 238. In certain other embodiments, the CH2 domain is an altered human IgG1, IgG2, IgG3, or IgG4 CH2 domain that comprises one or more amino acid mutations at positions 234-238, 255, 256, 257, 258, 290, 297, 318, 320, 322, 331, and 339. In certain other embodiments, the CH2 domain is an altered human IgG1, IgG2, IgG3, or IgG4 CH2 domain that comprises one or more amino acid mutations at positions 234, 235, 237, 318, 320 and 322.

In certain embodiments, the CH3 domain is an altered human IgG1, IgG2, IgG3, or IgG4 molecule that comprises an amino acid substitution or deletion at position 405 or 407.

In certain embodiments, the hinge of both the first and second single chain polypeptides is an immunoglobulin hinge region, such as an IgG1, IgG2, IgG3, IgG4, IgA1, IgA2, IgD, or IgE hinge.

In certain embodiments, the immunoglobulin hinge is a wild type immunoglobulin hinge. In certain other embodiments, the immunoglobulin hinge is an altered immunoglobulin hinge, such as those set forth in SEQ ID NOS:229-240.

In certain embodiments, the immunoglobulin hinge region is present at the amino terminal to the Fc region portion. In certain embodiments, the immunoglobulin hinge region is disposed between the binding domain and the immunoglobulin heterodimerization domain. In certain embodiments, the immunoglobulin hinge region is disposed between the immunoglobulin heterodimerization domain and the Fc region portion.

In certain embodiments, at least one of the first and second single chain polypeptide hinges is a C type lectin hinge region, such as a NKG2A or NKG2D peptide, or a derivative thereof.

In certain embodiments, the hinges of the first and second single chain polypeptides are identical. In certain other embodiments, the hinges of the first and second single chain polypeptides are different.

In certain embodiments, the first single chain polypeptide comprises amino acids 21-609 of SEQ ID NO:26, and the second single chain polypeptide comprises amino acids 21-363 of SEQ ID NO:137; the first single chain polypeptide comprises amino acids 21-716 of SEQ ID NO:46, and the second single chain polypeptide comprises amino acids 21-461 of SEQ ID NO:48; the first single chain polypeptide comprises amino acids 21-716 of SEQ ID NO:46 and the second single chain polypeptide comprises amino acids 21-461 of SEQ ID NO:64, the first single chain polypeptide comprises amino acids 21-716 of SEQ ID NO:62, and the second single chain polypeptide comprises amino acids 21-461 of SEQ ID NO:48; or the first single chain polypeptide comprises amino acids 21-716 of SEQ ID NO:62, and the second single chain polypeptide comprises amino acids 21-461 of SEQ ID NO:64; the first single chain polypeptide comprises SEQ ID NO:139, and the second single chain polypeptide comprises amino acids of 21-461 of SEQ ID NO:48; the first single chain polypeptide comprises SEQ ID NO:263, and the second single chain polypeptide comprises amino acids of 21-461 of SEQ ID NO:48; the first single chain polypeptide comprises SEQ ID NO:267, and the second single chain polypeptide comprises amino acids of 21-461 of SEQ ID NO:48, the first single chain polypeptide comprises SEQ ID

NO:769, and the second single chain polypeptide comprises SEQ ID NO:765; the first single chain polypeptide comprises SEQ ID NO:769, and the second single chain polypeptide comprises SEQ ID NO:766; the first single chain polypeptide comprises SEQ ID NO:769, and the second single chain polypeptide comprises SEQ ID NO:767; the first single chain polypeptide comprises SEQ ID NO:769, and the second single chain polypeptide comprises SEQ ID NO:768; the first single chain polypeptide comprises SEQ ID NO:781, and the second single chain polypeptide comprises SEQ ID

NO:778; and the first single chain polypeptide comprises SEQ ID NO:780; and the second single chain polypeptide comprises SEQ ID NO:779.

In certain embodiments, the first single chain polypeptide comprises amino acids 21-609 of SEQ ID NO:22, and the second single chain polypeptide comprises SEQ ID NO:91, 92, 193, 98, 99, 101, or 103, or amino acids 21-361 of SEQ ID NO:129, 131 or 133.

In certain embodiments, the first single chain polypeptide comprises amino acids 21-595 of SEQ ID NO:135, and the second single chain polypeptide comprises amino acids 21-361 of SEQ ID NO:24, 133 or 131.

In another aspect, the present disclosure provides a composition that comprises polypeptide heterodimers provided herein and a pharmaceutically acceptable excipient.

In another aspect, the present disclosure provides expression vectors capable of expressing the polypeptide heterodimers provided herein.

In another aspect, the present disclosure provides a host cell that comprises the expression vector capable of expressing the polypeptide heterodimers provided herein.

In a related aspect, the present disclosure provides a host cell that comprises first and second expression vectors capable of expressing the first and second single chain polypeptides, respectively, of the polypeptide heterodimers provided herein.

In another aspect, the present disclosure provides methods for making a polypeptide heterodimer, comprising (a) culturing host cells provided herein under conditions suitable to express two different single chain polypeptides, and (b) optionally isolating or purifying the heterodimers formed from the first and second single chain polypeptides from the culture.

In another aspect, the present disclosure provides methods for reducing T cell activation, comprising administering to a patient in need thereof an effective amount of a polypeptide heterodimer as provided herein, wherein the binding domain of the polypeptide heterodimer specifically binds CD28.

In another aspect, the present disclosure provides methods for inhibiting growth of a solid malignancy, comprising administering to a patient in need thereof an effective amount of a polypeptide heterodimer provided herein, wherein the binding domain of the polypeptide heterodimer specifically binds EGFR, ErbB3, ErbB4, c-Met, RON, CEACAM6, EphA2, IGF1R, GHRHR, GHR, VEGFR1, VEGFR2, VEGFR3, CD44v6, CD151, TGFBR2, IL6R, gp130, TNFR2, PD1, TWEAK-R, OSMRbeta, Patched-1, Frizzled, or Robo1. In certain embodiments, the method further comprises administering to a patient in need thereof a chemotherapeutic agent or ionizing radiation.

In another aspect, the present disclosure provides methods for treating an autoimmune or inflammatory condition, comprising administering to a patient in need thereof an effective amount of a polypeptide heterodimer provided herein, wherein the binding domain of the polypeptide heterodimer specifically binds TGFBR2, TGFBR1, IL6R, gp130, TNFR1, TNFR2, PD1, HVEM, OX40, CD40, CD137, TWEAK-R, LTbetaR, LIFRbeta, OSMRbeta, CD3, TCRalpha, TCRbeta, CD19, CD28, CD80, CD81, CD86, TLR7, or TLR9.

In certain embodiments, the methods for using the polypeptide heterodimers provided herein may further comprise administering to a patient in need thereof a second active agent, such as a second polypeptide heterodimer, or a monoclonal antibody, or an immunoglobulin-derived fusion protein.

### BRIEF DESCRIPTION OF THE DRAWINGS

FIGS. 1A and 1B show a schematic of (A) a class 1 polypeptide heterodimer (Interceptor) (i.e., having a binding domain at the amino terminus) and (B) a class 2 Interceptor (i.e., having a binding domain at the carboxyl terminus), as described in Example 1.

FIGS. 2A-2C show a schematic of various exemplary class 1 Interceptors, including (A) one having a CH1-Cκ pair at the amino terminus (X0124), (B) one having a CH1-Cκ pair at the carboxyl terminus (X0126), and (C) one having a CH1-Cκ pair at the amino terminus and another CH1-Cκ pair at the carboxyl terminus (X0128).

FIG. 3 shows that when X0124 was expressed by co-transfecting X0112 and X0113, wherein only the heterodimer and homodimer of the light chain were expressed. “NR” stands for “Non-Reduced,” and “R” stands for “Reduced.”

FIG. 4 shows analysis of X0124 by mass spectrometry, indicating that the light chain homodimer and heterodimer were expressed at approximately 1:1 ratio with no evidence of the presence of the heavy chain homodimer.

FIG. 5 shows that when X0126 was expressed by co-transfecting X0114 and X0115, wherein only the heterodimer and homodimer of the light chain were expressed. “NR” stands for “Non-Reduced,” and “R” stands for “Reduced.”

FIG. 6 shows that when X0128 was expressed by co-transfecting X0120 and X0121, wherein only the heterodimer and homodimer of the light chain were expressed. “NR” stands for “Non-Reduced,” and “R” stands for “Reduced.”

FIG. 7A shows that when X0125 was expressed by co-transfecting X0116 and X0117, wherein in addition to heterodimer and homodimer of the light chain, monomers of the light and heavy chains were expressed. “NR” stands for “Non-Reduced,” and “R” stands for “Reduced.”

FIG. 7B shows that when X0127 was expressed by co-transfecting X0119 and X0118, wherein in addition to heterodimer and homodimer of the light chain, monomers of the light and heavy chains were also expressed. “NR” stands for “Non-Reduced,” and “R” stands for “Reduced.”

FIG. 8A shows that when X0138 was expressed by co-transfecting X0137 and X0136, wherein in addition to heterodimer and homodimer of the light chain, monomer of the light chain was also expressed. “NR” stands for “Non-Reduced,” and “R” stands for “Reduced.”

FIG. 8B shows that when X0141 was expressed by co-transfecting X0140 and X0139, wherein in addition to heterodimer and homodimer of the light chain, monomer of the light chain was also expressed.

FIG. 9 shows ELISA results in which a plate coated with CD28 mIg was contacted with Interceptors specific for CD28 (X0124, X0125, X0126, X0127, X0128, and X0129), an anti-CD28 SMIP protein (M0039), or the negative control of a homodimer of light chain X0113, and then binding was detected with anti-human IgG HRP.

FIG. 10 shows ELISA results in which a plate coated with CD28 mIg was contacted with Interceptors specific for CD28 (X00124, X0125, X0126, X0127, X0128, and X0129), an anti-CD28 SMIP protein (M0039), or the negative control of a homodimer of light chain X0113, and then binding was detected with anti-human Cκ HRP.

FIG. 11 shows cation exchange chromatography used to separate the heterodimer X0124 from the homodimer of the light chain.

FIG. 12 is SDS-PAGE analysis of X0124 that shows a higher heterodimer content after repurification with the cation exchange column. “NR” stands for “Non-Reduced,” and “R” stands for “Reduced.”

FIG. 13 is SDS-PAGE analysis of X0124 and X0126 Interceptors before and after protein L purification, showing that greater than 95% heterodimer was obtained after the second step protein L purification. “NR” stands for “Non-Reduced,” and “Red” stands for “Reduced.”

FIG. 14A is a schematic of X0142 in which Cκ of X0124 was replaced with a Cλ.

FIG. 14B is a schematic of X0143 in which Cκ of X0126 was replaced with a Cλ.

FIG. 15 shows SDS-PAGE results of X0142 and X0143, showing both heterodimer and light chain homodimer are formed when a Cλ heterodimerization domain is used in place of Cκ. “NR” stands for “Non-Reduced,” and “Red” stands for “Reduced.”

FIG. 16 shows a schematic representation of expression of X0130 alone, expression of X0131 alone, and co-expression of X0130 (long chain) and X0131 (short chain) that produced X0132. Expression of X0130 alone yielded no protein and expression of X0131 yielded little protein, whereas co-expression of X0130 and X0131 (especially at a 2:1 ratio) yielded pure heterodimer.

FIG. 17 shows SDS-PAGE results of X0132 using 1:1 X0130 (long) and X0131 (short) ratio or 2:1 X0130 and X0131 ratio for transfection.

FIG. 18 shows the mass spectra of X0132, which demonstrates that 100% heterodimer is formed.

FIG. 19 shows schematic representations of exemplary Interceptors with two pairs of Cκ/Cλ-CH1 combinations, X0132, X0166, X0165 and X0149.

FIG. 20 shows SDS-PAGE results of Interceptors X0132, X0166, X0165 and X0149 with Cκ-Ch1 and Cλ-CH1 combinations, demonstrating that heterodimers were greater than 90% pure.

FIG. 21 shows SEC results of Interceptors X0132, X0165, X0166 and X0145 with different Cκ-CH1 and Cλ-CH1 combinations.

FIG. 22 shows binding of selected Interceptors (X0124, X0128 and X0132) on Jurkat T cell lines.

FIG. 23 shows that anti-CD28 in different molecular formats blocked primary MLR.

FIG. 24 shows that Interceptors block secondary MLR.

FIG. 25 shows that bivalent anti-CD28 molecules (SMIP and 2E12 Mab), but not Interceptors, synergize with a suboptimal concentration of PMA in stimulating purified human T cells.

FIGS. 26A-26D show direct binding to immobilized CD28 by (A) 2E12 antibody fragment (Fab), (B) 2E12 single-chain variable fragment (scFv), and 2E12 heterodimeric monovalent polypeptides (C) X0124 and (D) X0132, with response units (Ru) plotted against time.

FIGS. 27A-27B show binding of bivalent 2E12 binding polypeptides. 1:1 binding of directly immobilized CD28 by 2E12 monoclonal antibody (mAb) (FIG. 27A), and 2E12 SMIP protein (M0039) (FIG. 27B), with response units (Ru) plotted against time (top).

FIG. 28 shows SDS-PAGE results of X0171. “NR” stands for “Non-Reduced,” and “Red” stands for “Reduced.”

FIG. 29 shows a mass spectrum of Interceptor X0171 that demonstrates that the heterodimer is the predominant species.

FIG. 30 shows cation exchange chromatography of homodimer/heterodimer mixtures obtained after initial protein A affinity purification. Individual experimental traces are shown overlaid in a stack plot; individual absorbances have not been scaled. Individual peaks isolated and shown to be heterodimeric are labeled with an asterisk (*).

FIG. 31 shows cation exchange chromatography of predominantly heterodimeric proteins obtained after either initial protein A affinity purification (X0132, X0171, X0172) or after secondary protein L purification (X0124, compare to FIG. 30). Individual experimental traces are shown overlaid in a stack plot; individual absorbances have not been scaled. Heterodimeric species are labeled with an asterisk (*).

FIG. 32 shows crystal structure of Ck-Ck overlaid with Ck-CH1.

FIG. 33 shows the hydrogen bond network found in the Ck-Ck interface.

FIG. 34 shows the seven residues involved in the Hydrogen bonding at the Ck-CK interface.

FIG. 35 shows SDS-PAGE results of single residue alanine scanning on X0124. “NR” stands for “Non-Reduced,” and “Red” stands for “Reduced.”

FIG. 36 shows SDS-PAGE results of double alanine scanning of selected Cκ residues. “NR” stands for “Non-Reduced,” and “Red” stands for “Reduced.”

FIG. 37 shows SDS-PAGE results of triple alanine scanning of selected Cκ residues. “NR” stands for “Non-Reduced,” and “Red” stands for “Reduced.”

FIG. 38 shows SDS-PAGE results of Interceptors containing mutations that introduce bulky amino acid side chains at four different positions: 52, 56, 68 and 70.

FIG. 39 shows SDS-PAGE results of Interceptors with combinations of bulky side chain amino acid mutations and alanine mutations introduced at selected Cκ residues.

FIG. 40 shows SDS-PAGE results of Interceptors with additional combinations of bulky side chain amino acid mutations and alanine mutations introduced at selected Cκ residues.

FIG. 41 shows SDS-PAGE analysis under non-reducing conditions of

Interceptor with combinations of bulky side chain amino acid mutations and alanine mutations introduced at positions 29, 30, 55 and 70. The left panel shows results of Interceptors with Cκ heterdimerization domains near the N-terminus of short chains (i.e., do not contain an scFv). The right panel shows results of Interceptors with Cκ heterodimerization domains near the C-terminus of the short chain.

FIG. 42 shows anti-c-Met (5D5) SMIP and Interceptor activity on HT-29 cells in a c-Met phosphorylation ELISA assay.

### DETAILED DESCRIPTION

The present disclosure provides polypeptide heterodimers formed between two different single chain polypeptides via natural heterodimerization of an immunoglobulin CH1 region and an immunoglobulin light chain constant region (CL). The longer chain of a heterodimer has a binding domain that specifically binds a target (e.g., a receptor or a ligand). In addition, both chains of a heterodimer further each comprise an Fc region portion (e.g., immunoglobulin CH2 and/or CH3 domains). The present disclosure also provides nucleic acids, vectors, host cells and methods for making polypeptide heterodimers as well as methods for using such polypeptide heterodimers, such as in reducing T cell activation, inhibiting solid malignancy growth, and treating autoimmune or inflammatory conditions.

The heterodimerization technology described herein has one or more of the following advantages: (1) minimal immunogenicity of the polypeptide heterodimers because the dimers are formed via natural heterodimerization of an immunoglobulin CH1 region and an immunoglobulin CL region; (2) efficient production and purification of polypeptide heterodimers of the present disclosure is possible by co-expressing the two different single chain polypeptides, as shown in the examples; (3) the ability to mediate Fc effector functions (e.g., CDC, ADCC, ADCP), which can be modulated up or down by mutagenesis, and a longer serum half life because each chain of a polypeptide heterodimer according to the present disclosure has an Fc region portion (e.g., immunoglobulin CH2 and CH3 domains); and (4) polypeptide heterodimers of the present disclosure having a size that is typically smaller than an antibody molecule, which can allow for better tissue penetration, such as into a solid malignancy.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents defines a term that contradicts that term's definition in the application, the definition that appears in this application controls.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±20% of the indicated range, value, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual heterodimers derived from various combinations of the components (e.g., domains, regions, hinges and linkers) described herein, are disclosed by the present application to the same extent as if each single chain polypeptide or heterodimer were set forth individually. Thus, selection of particular components of individual single chain polypeptides or heterodimers is within the scope of the present disclosure.

As used herein, a protein “consists essentially of” several domains (e.g., a binding domain that specifically binds a target, a hinge, an immunoglobulin heterodimerization domain, and an Fc region constant domain portion) if the other portions of the protein (e.g., amino acids at the amino- or carboxy-terminus or between two domains), in combination, contribute to at most 20% (e.g., at most 15%, 10%, 8%, 6%, 5%, 4%, 3%, 2% or 1%) of the length of the protein and do not substantially affect (i.e., do not reduce the activity by more than 50%, such as more than 40%, 30%, 25%, 20%, 15%, 10%, or 5%) the activities of various domains (e.g., the target binding affinity of the binding domain, the activities of the Fc region portion, and the capability of the heterodimerization domain in facilitating heterodimerization). In certain embodiments, a protein (e.g., a single chain polypeptide) consists essentially of a binding domain that specifically binds a target, an immunoglobulin heterodimerization domain, a hinge, and an Fc region portion may comprise junction amino acids at the amino- and/or carboxy-terminus of the protein or between two different domains (e.g., between the binding domain and the immunoglobulin heterodimerization domain, between the immunoglobulin heterodimerization domain and the hinge, and/or between the hinge and the Fc region portion).

A “polypeptide heterodimer,” “heterodimer,” or “Interceptor,” as used herein, refers to a dimer formed from two different single chain polypeptides, comprising at least one chain longer (long chain) than the other (short chain). This term does not include an antibody formed from four single chain polypeptides (i.e., two light chains and two heavy chains). A “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis).

A “single chain polypeptide” is a single, linear and contiguous arrangement of covalently linked amino acids. It does not include two polypeptide chains that link together in a non-linear fashion, such as via an interchain disulfide bond (e.g., a half immunoglobulin molecule in which a light chain links with a heavy chain via a disulfide bond). In certain embodiments, a single chain polypeptide may have or form one or more intrachain disulfide bonds.

An “immunoglobulin heterodimerization domain,” as used herein, refers to an immunoglobulin domain (“first immunoglobulin heterodimerization domain”) that preferentially interacts or associates with a different immunoglobulin domain (“second immunoglobulin heterodimerization domain”) wherein the interaction of the different heterodimerization domains substantially contributes to or efficiently promotes heterodimerization (i.e., the formation of a dimer between two different polypeptides, which is also referred to as a heterodimer). Representative immunoglobulin heterodimerization domains of the present disclosure include an immunoglobulin CH1 region, an immunoglobulin CL region (e.g., Cκ or Cλ isotypes), or derivatives thereof, as provided herein. In certain embodiments, a polypeptide heterodimer comprises (i) a single chain polypeptide (“first single chain polypeptide”) having a first immunoglobulin heterodimerization domain and (ii) another single chain polypeptide (“second single chain polypeptide”) having a second immunoglobulin heterodimerization domain that is not the same as the first immunoglobulin heterodimerization domain, wherein the first and second immunoglobulin heterodimerization domains substantially contribute to or efficiently promote formation of the polypeptide heterodimer. The interaction(s) between the first and second heterodimerization domains substantially contributes to or efficiently promotes the heterodimerization of the first and second single chain polypeptides if there is a statistically significant reduction in the dimerization between the first and second single chain polypeptides in the absence of the first heterodimerization domain and/or the second heterodimerization domain. In certain embodiments, when the first and second single chain polypeptides are co-expressed, at least about 60%, for instance, at least about 60% to about 70%, at least about 70% to about 80%, at least about 80% to about 90%, 91%, 92%, 93%, 94%, 95%, 96%, 97%, 98%, 99%, or 100%, and at least about 90% to about 92%, 93%, 94%, 95%, 96%, 97%, 98%, or 99% of the first and second single chain polypeptides form heterodimers with each other.

A “binding domain” or “binding region,” as used herein, refers to a protein, polypeptide, oligopeptide, or peptide that possesses the ability to specifically recognize and bind to a target (e.g., CD3, CD28, c-Met, RON). A binding domain includes any naturally occurring, synthetic, semi-synthetic, or recombinantly produced binding partner for a biological molecule or another target of interest. Exemplary binding domains include single chain antibody variable regions (e.g., domain antibodies, sFv, scFv, Fab), receptor ectodomains (e.g., c-Met, RON), or ligands (e.g., cytokines, chemokines). A variety of assays are known for identifying binding domains of the present disclosure that specifically bind a particular target, including Western blot, ELISA, and Biacore analysis.

A binding domain and a fusion protein thereof “specifically binds” a target if it binds the target with an affinity or Ka (i.e., an equilibrium association constant of a particular binding interaction with units of 1/M) equal to or greater than 10⁵ M⁻¹, while not significantly binding other components present in a test sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10^{11 M}^{-1,} at least 10^{12 M}⁻¹ or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

“T cell receptor” (TCR) is a molecule found on the surface of T cells that, along with CD3, is generally responsible for recognizing antigens bound to major histocompatibility complex (MHC) molecules. It consists of a disulfide-linked heterodimer of the highly variable α and β chains in most T cells. In other T cells, an alternative receptor made up of variable γ and δ chains is expressed. Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see, Abbas and Lichtman, Cellular and Molecular Immunology (5th Ed.), Editor: Saunders, Philadelphia, 2003; Janeway et al., Immunobiology: The Immune System in Health and Disease, 4^{th} Ed., Current Biology Publications, p 148, 149, and 172, 1999). TCR as used in the present disclosure may be from various animal species, including human, mouse, rat, or other mammals.

“CD3” is known in the art as a multi-protein complex of six chains (see, Abbas and Lichtman, 2003; Janeway et al., p 172 and 178, 1999). In mammals, the complex comprises a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. It is believed the ITAMs are important for the signaling capacity of a TCR compelx. CD3 as used in the present disclosure may be from various animal species, including human, mouse, rat, or other mammals.

“TCR complex,” as used herein, refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain.

“A component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CDγ, CD3δ, and two CD3ε chains).

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

For example, the terms “VL” and “VH” refer to the variable binding region from an antibody light and heavy chain, respectively. The variable binding regions are made up of discrete, well-defined sub-regions known as “complementarity determining regions” (CDRs) and “framework regions” (FRs).The term “CL” refers to an “immunoglobulin light chain constant region” or a “light chain constant region,” i.e., a constant region from an antibody light heavy chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 of the heavy chain linked to the light chain via an inter-chain disulfide bond.

As used herein, “an Fc region constant domain portion” or “Fc region portion” refers to the heavy chain constant region segment of the Fc fragment (the “fragment crystallizable” region or Fc region) from an antibody, which can include one or more constant domains, such as CH2, CH3, CH4, or any combination thereof. In certain embodiments, an Fc region portion includes the CH2 and CH3 domains of an IgG, IgA, or IgD antibody and any combination thereof, or the CH3 and CH4 domains of an IgM or IgE antibody and any combination thereof. In one embodiment, the CH2CH3 or the CH3CH4 structures are from the same antibody isotype, such as IgG, IgA, IgD, IgE, or IgM. By way of background, the Fc region is responsible for the effector functions of an immunoglobulin, such as ADCC (antibody-dependent cell-mediated cytotoxicity), ADCP (antibody-dependent cellular phagocytosis), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al., Nature, 337:525 (1989)). In certain embodiments, an Fc region portion found in polypeptide heterodimers of the present disclosure will be capable of mediating one or more of these effector functions.

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2.

According to crystallographic studies, an IgG hinge domain can be functionally and structurally subdivided into three regions: the upper, the core or middle, and the lower hinge regions (Shin et al., Immunological Reviews 130:87 (1992)). Exemplary upper hinge regions include EPKSCDKTHT (SEQ ID NO:227) as found in IgG1, ERKCCVE (SEQ ID NO:211) as found in IgG2, ELKTPLGDTT HT (SEQ ID NO:245) or EPKSCDTPPP (SEQ ID NO:246) as found in IgG3, and ESKYGPP (SEQ ID NO:247) as found in IgG4. Exemplary middle or core hinge regions include CPPCP (SEQ ID NO:228) as found in IgG1 and IgG2, CPRCP (SEQ ID NO:248) as found in IgG3, and CPSCP (SEQ ID NO:249) as found in IgG4. While IgG1, IgG2, and IgG4 antibodies each appear to have a single upper and middle hinge, IgG3 has four in tandem—one being ELKTPLGDTTHTCPRCP (SEQ ID NO:250) and three being EPKSCDTPPP CPRCP (SEQ ID NO:251).

IgA and IgD antibodies appear to lack an IgG-like core region, and IgD appears to have two upper hinge regions in tandem (see SEQ ID NOS:222 and 252).

Exemplary wild type upper hinge regions found in IgA1 and IgA2 antibodies are set forth in SEQ ID NOS:215 and 216.

IgE and IgM antibodies, in contrast, lack a typical hinge region and instead have a CH2 domain with hinge-like properties. Exemplary wild-type CH2 upper hinge-like sequences of IgE and IgM are set forth in SEQ ID NO:253 (VCSRDFTPPTVKILQSSSDGGGHFPPTIQLLCLVSGYTPGTINITWLEDG QVMDVDLSTASTTQEGELASTQSELTLSQKHWLSDRTYTCQVTYQGHTFE DSTKKCalif,) and SEQ ID NO:254 (VIAELPPKVSVFVPPRDGFFGNPRKSKLIC QATGFSPRQIQVSWLREGKQVGSGVTTDQVQAEAKESGPTTYKVTSTLTI KESDWLGQSMFTCRVDHRGLTFQQNASSMCVP), respectively.

As used herein, a “hinge region” or a “hinge” refers to (a) an immunoglobulin hinge region (made up of, for example, upper and core regions) or a functional variant thereof, including wild type and altered immunoglobulin hinges, (b) a lectin interdomain region or a functional variant thereof, (c) a cluster of differentiation (CD) molecule stalk region or a functional variant thereof, or (d) a portion of a cell surface receptor (interdomain region) that connects immunoglobulin V-like or immunoglobulin C-like domains.

As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human. In certain embodiments, the wild type immunoglobulin hinge region comprises a human IgG hinge region. Exemplary human wild type immunoglobulin hinge regions are set forth in SEQ ID NOS:215 (IgA1 hinge), 216 (IgA2 hinge), 217 (IgD hinge), 218 (IgG1 hinge), 219 (IgG2 hinge), 220 (IgG3 hinge) and 221 (IgG4 hinge).

An “altered wild type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as set forth in SEQ ID NOS:228, 248, or 249.

A “peptide linker” refers to an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of about five to about 35 amino acids, for instance, about 15 to about 25 amino acids.

“Junction amino acids” or “junction amino acid residues” refer to one or more (e.g., about 2-10) amino acid residues between two adjacent regions or domains of a single chain polypeptide, such as between a hinge and an adjacent Fc region portion or between a hinge and an adjacent binding domain or between a peptide linker that links two immunoglobulin variable domains and an adjacent immunoglobulin variable domain. Junction amino acids may result from the construct design of a single chain polypeptide (e.g., amino acid residues resulting from the use of a restriction enzyme site during the construction of a nucleic acid molecule encoding a single chain polypeptide).

A “linker between CH3 and CH1 or CL” refers to one or more (e.g., about 2-12) amino acid residues between the C-terminus of CH3 (e.g., a wild type CH3 or a mutated CH3) and the N-terminus of CH1 or CL (e.g., Ck).

A “wild type immunoglobulin region” or “wild type immunoglobulin domain” refers to a naturally occurring immunoglobulin region or domain (e.g., a naturally occurring VL, VH, hinge, CL, CH1, CH2, CH3, or CH4) from various immunoglobulin classes or subclasses (including, for example, IgG1, IgG2, IgG3, IgG4, IgA1, IgA2, IgD, IgE, and IgM) and from various species (including, for example, human, sheep, mouse, rat, and other mammals). Exemplary wild type human CH1 regions are set forth in SEQ ID NOS:114, 186-192 and 194, wild type human Cκ region in SEQ ID NO:112, wild type human Cλ regions in SEQ ID NO:113 and 224-226, wild type human CH2 domains in SEQ ID NOS:115, 195-201 and 203, wild type human CH3 domains in SEQ ID NOS:116, 204-210 and 212, and wild type human CH4 domains in SEQ ID NO:213 and 214.

An “altered immunoglobulin region” or “altered immunoglobulin domain” refers to an immunoglobulin region with a sequence identity to a wild type immunoglobulin region or domain (e.g., a wild type VL, VH, hinge, CL, CH1, CH2, CH3, or CH4) of at least 75% (e.g., 80%, 82%, 84%, 86%, 88%, 90%, 91%, 92%, 93%, 94%, 95%, 96%, 97%, 98%, 99%, or 99.5%). For example, an “altered immunoglobulin CH1 region” or “altered CH1 region” refers to a CH1 region with a sequence identity to a wild type immunoglobulin CH1 region (e.g., a human CH1) of at least 75% (e.g., 80%, 82%, 84%, 86%, 88%, 90%, 91%, 92%, 93%, 94%, 95%, 96%, 97%, 98%, 99%, or 99.5%). Similarly, an “altered immunoglobulin CH2 domain” or “altered CH2 domain” refers to a CH2 domain with a sequence identity to a wild type immunoglobulin CH1 region (e.g., a human CH2) of at least 75% (e.g., 80%, 82%, 84%, 86%, 88%, 90%, 91%, 92%, 93%, 94%, 95%, 96%, 97%, 98%, 99%, or 99.5%).

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values are generated by the NCBI BLAST2.0 software as defined by Altschul et al. (1997) “Gapped BLAST and PSI-BLAST: a new generation of protein database search programs,” Nucleic Acids Res. 25:3389-3402, with the parameters set to default values.

In certain embodiments, an altered immunoglobulin domain only contains conservative amino acid substitutions of a wild type immunoglobulin domain. In certain other embodiments, an altered immunoglobulin domain only contains non-conservative amino acid substitutions of a wild type immunoglobulin domain. In yet other embodiments, an altered immunoglobulin domain contains both conservative and non-conservative amino acid substitutions.

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:N.Y. (1975), pp.71-77; Lewin, Genes IV, Oxford University Press, NY and Cell Press, Cambridge, Mass. (1990), p. 8). In certain embodiments, a conservative substitution includes a leucine to serine substitution.

As used herein, the term “derivative” refers to a modification of one or more amino acid residues of a peptide by chemical or biological means, either with or without an enzyme, e.g., by glycosylation, alkylation, acylation, ester formation, or amide formation. Generally, a “derivative” differs from an “analogue” in that a parent polypeptide may be the starting material to generate a “derivative,” whereas the parent polypeptide may not necessarily be used as the starting material to generate an “analogue.” A derivative may have different chemical, biological or physical properties of the parent polypeptide. For example, a derivative may be more hydrophilic or it may have altered reactivity (e.g., a CDR having an amino acid change that alters its affinity for a target) as compared to the parent polypeptide.

As used herein, unless otherwise provided, a position of an amino acid residue in a variable region of an immunoglobulin molecule is numbered according to the Kabat numbering convention (Kabat, Sequences of Proteins of Immunological Interest, 5^{th} ed. Bethesda, Md.: Public Health Service, National Institutes of Health (1991)), and a position of an amino acid residue in a constant region of an immunoglobulin molecule is numbered according to EU nomenclature (Ward et al., 1995 Therap. Immunol. 2:77-94).

A “receptor” is a protein molecule present in the plasma membrane or in the cytoplasm of a cell to which a signal molecule (i.e., a ligand, such as a hormone, a neurotransmitter, a toxin, a cytokine) may attach. The binding of the single molecule to the receptor results in a conformational change of the receptor, which ordinarily initiates a cellular response. However, some ligands merely block receptors without inducing any response (e.g., antagonists). Some receptor proteins are peripheral membrane proteins, many hormone and neurotransmitter receptors are transmembrane proteins that embedded in the phospholipid bilayer of cell membranes, and another major class of receptors are intracellular proteins such as those for steroid and intracrine peptide hormone receptors.

“Treatment,” “treating” or “ameliorating” refers to either a therapeutic treatment or prophylactic/preventative treatment. A treatment is therapeutic if at least one symptom of disease in an individual receiving treatment improves or a treatment may delay worsening of a progressive disease in an individual, or prevent onset of additional associated diseases.

A “therapeutically effective amount (or dose)” or “effective amount (or dose)” of a specific binding molecule or compound refers to that amount of the compound sufficient to result in amelioration of one or more symptoms of the disease being treated in a statistically significant manner. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously.

The term “pharmaceutically acceptable” refers to molecular entities and compositions that do not produce allergic or other serious adverse reactions when administered using routes well known in the art.

A “patient in need” refers to a patient at risk of, or suffering from, a disease, disorder or condition that is amenable to treatment or amelioration with a polypeptide heterodimer or a composition thereof provided herein.

The term “immunoglobulin-derived fusion protein,” as used herein, refers to a fusion protein that comprises at least one immunoglobulin region, such as a VL, VH, CL, CH1, CH2, CH3, and CH4 domain. The immunoglobulin region may be a wild type immunoglobulin region or an altered immunoglobulin region. Exemplary immunoglobulin-derived fusion proteins include single chain variable antibody fragment (scFv) (see, e.g., Huston et al., Proc. Natl. Acad. Sci. USA 85: 5879-83, 1988), small modular immunopharmaceutical (SMIP™) proteins (see, U.S. Patent Publication Nos. 2003/0133939, 2003/0118592, and 2005/0136049), PIMS proteins (see, PCT Application Publication No. WO 2009/023386), and multi-functional binding proteins (such as SCORPION™ and Xceptor proteins) (see, PCT Application Publication No. WO 2007/146968, U.S. Patent Application Publication No. 2006/0051844, and U.S. Pat. No. 7,166,707).

Additional definitions are provided throughout the present disclosure.

### Polypeptide Heterodimers

In one aspect, the present disclosure provides a polypeptide heterodimer formed by the association of two different single chain polypeptides. The first or long single chain polypeptide comprises, consists essentially of, or consists of a binding domain that specifically binds a target, a hinge, a first immunoglobulin heterodimerization domain, and an Fc region portion, whereas the second or short single chain polypeptide comprises, consists essentially of, or consists of a hinge, a second immunoglobulin heterodimerization domain, an Fc region portion, and does not comprise a target binding domain. The hinge in the first single chain polypeptide may or may not be the same as the hinge in the second single chain polypeptide. The first immunoglobulin heterodimerization domain in the first single chain polypeptide is different from the second immunoglobulin heterodimerization domain in the second single chain polypeptide. The Fc region portion of the first single chain polypeptide may be the same as the Fc region portion of the second single chain polypeptide. The individual components of the polypeptide heterodimers of the present disclosure are described in detail herein.

### Binding Domains

As indicated above, a long single chain polypeptide of the polypeptide heterodimer of the present disclosure comprises a binding domain that specifically binds a target. Binding of a target by the binding domain may block the interaction between the target (e.g., a receptor or a ligand) and another molecule, and thus interfere, reduce or eliminate certain functions of the target (e.g., signal transduction).

A binding domain may be any peptide that specifically binds a target of interest. Sources of binding domains include antibody variable regions from various species (which can be formatted as antibodies, sFvs, scFvs, Fabs, or soluble VH domain or domain antibodies), including human, rodent, avian, and ovine. Additional sources of binding domains include variable regions of antibodies from other species, such as camelid (from camels, dromedaries, or llamas; Ghahroudi et al. (1997) FEBS Letters 414(3):521-526; Vincke et al. (2009) Journal of Biological Chemistry (2009) 284:3273-3284; Hamers-Casterman et al. (1993) Nature, 363:446 and Nguyen et al. (1998) J. Mol. Biol., 275:413), nurse sharks (Roux et al. (1998) Proc. Nat'l. Acad. Sci. (USA) 95:11804), spotted ratfish (Nguyen et al. (2002) Immunogenetics, 54:39), or lamprey (Herrin et al., (2008) Proc. Nat'l. Acad. Sci. (USA) 105:2040-2045 and Alder et al. (2008) Nature Immunology 9:319-327). These antibodies can apparently form antigen-binding regions using only heavy chain variable region, i.e., these functional antibodies are homodimers of heavy chains only (referred to as “heavy chain antibodies”) (Jespers et al. (2004) Nature Biotechnology 22:1161-1165; Cortez-Retamozo et al. (2004) Cancer Research 64:2853-2857; Baral et al. (2006) Nature Medicine 12:580-584, and Barthelemy et al. (2008) Journal of Biological Chemistry 283:3639-3654).

An alternative source of binding domains of this disclosure includes sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), ankyrin repeat proteins (Binz et al. (2003) Journal of Molecular Biology 332:489-503 and Binz et al. (2004) Nature Biotechnology 22(5):575-582), fibronectin binding domains (Richards et al. (2003) Journal of Molecular Biology 326:1475-1488; Parker et al. (2005) Protein Engineering Design and Selection 18(9):435-444 and Hackel et al. (2008) Journal of Molecular Biology 381:1238-1252), cysteine-knot miniproteins (Vita et al. (1995) Proc. Nat'l. Acad. Sci. (USA) 92:6404-6408; Martin et al. (2002) Nature Biotechnology 21:71-76 and Huang et al. (2005) Structure 13:755-768), tetratricopeptide repeat domains (Main et al. (2003) Structure 11:497-508 and Cortajarena et al. (2008) ACS Chemical Biology 3:161-166), leucine-rich repeat domains (Stumpp et al. (2003) Journal of Molecular Biology 332:471-487), lipocalin domains (see, e.g., WO 2006/095164, Beste et al. (1999) Proc. Nat'l. Acad. Sci. (USA) 96:1898-1903 and Schonfeld et al. (2009) Proc. Nat'l. Acad. Sci. (USA) 106:8198-8203), V-like domains (see, e.g., US Patent Application Publication No. 2007/0065431), C-type lectin domains (Zelensky and Gready (2005) FEBS J. 272:6179; Beavil et al. (1992) Proc. Nat'l. Acad. Sci. (USA) 89:753-757 and Sato et al. (2003) Proc. Nat'l. Acad. Sci. (USA) 100:7779-7784), mAb² or Fcab™ (see, e.g., PCT Patent Application Publication Nos. WO 2007/098934; WO 2006/072620), or the like (Nord et al. (1995) Protein Engineering 8(6):601-608; Nord et al. (1997) Nature Biotechnology 15:772-777; Nord et al. (2001) European Journal of Biochemistry 268(15):4269-4277 and Binz et al. (2005) Nature Biotechnology 23:1257-1268).

Exemplary anti-CD3 antibodies from which the binding domain of this disclosure may be derived include Cris-7 monoclonal antibody (Reinherz, E. L. et al. (eds.), Leukocyte typing II., Springer Verlag, New York, (1986)), BC3 monoclonal antibody (Anasetti et al. (1990) J. Exp. Med. 172:1691), OKT3 (Ortho multicenter Transplant Study Group (1985) N. Engl. J. Med. 313:337) and derivatives thereof such as OKT3 ala-ala (Herold et al. (2003) J. Clin. Invest. 11:409), visilizumab (Carpenter et al. (2002) Blood 99:2712), and 145-2C11 monoclonal antibody (Hirsch et al. (1988) J. Immunol. 140: 3766). An exemplary anti-TCR antibody is H57 monoclonal antibody (Lavasani et al. (2007) Scandinavian Journal of Immunology 65:39-47).

Binding domains of this disclosure can be generated as described herein or by a variety of methods known in the art (see, e.g., U.S. Pat. Nos. 6,291,161 and 6,291,158). For example, binding domains of this disclosure may be identified by screening a Fab phage library for Fab fragments that specifically bind to a target of interest (see Hoet et al. (2005) Nature Biotechnol. 23:344). Additionally, traditional strategies for hybridoma development using a target of interest as an immunogen in convenient systems (e.g., mice, HuMAb mouse®, TC mouse™, KMmouse®, llamas, chicken, rats, hamsters, rabbits, etc.) can be used to develop binding domains of this disclosure.

In some embodiments, a binding domain is a single chain Fv fragment (scFv) that comprises VH and VL regions specific for a target of interest. In certain embodiments, the V_{H} and V_{L} domains are human. Exemplary VH regions include the VH region of 2E12 (anti-CD28) scFv as set forth in SEQ ID NO:106, the VH region of P2C2 (anti-CD79b) scFv as set forth in SEQ ID NO:184, the VH region of 5D5 (anti-c-Met) scFv as set forth in SEQ ID NO:258. Exemplary VL domains are the VL region of 2E12 scFv as set forth in SEQ ID NO:107, the VL region of P2C2 scFv as set forth in SEQ ID NO:182, the VL region of 5D5 (anti-c-Met) scFv as set forth in SEQ ID NO:259.

In certain embodiments, a binding domain comprises or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (V_{L}) (e.g., SEQ ID NOS:107, 182 and 259) or to a heavy chain variable region (V_{H}) (e.g., SEQ ID NOS:106, 184 and 258), or both, wherein each CDR comprises zero changes or at most one, two, or three changes, from a monoclonal antibody or fragment or derivative thereof that specifically binds to target of interest (e.g., c-Met, RON, CD28, CD79b, HER3).

In certain embodiments, a binding domain VH region of the present disclosure can be derived from or based on a VH of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of a known monoclonal antibody. The insertion(s), deletion(s) or substitution(s) may be anywhere in the VH region, including at the amino- or carboxy-terminus or both ends of this region, provided that each CDR comprises zero changes or at most one, two, or three changes and provided a binding domain containing the modified VH region can still specifically bind its target with an affinity similar to the wild type binding domain.

In further embodiments, a VL region in a binding domain of the present disclosure is derived from or based on a VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known monoclonal antibody. The insertion(s), deletion(s) or substitution(s) may be anywhere in the VL region, including at the amino- or carboxy-terminus or both ends of this region, provided that each CDR comprises zero changes or at most one, two, or three changes and provided a binding domain containing the modified V_{L} region can still specifically bind its target with an affinity similar to the wild type binding domain.

The VH and VL domains may be arranged in either orientation (i.e., from amino-terminus to carboxyl terminus, VH-VL or VL-VH) and may be joined by an amino acid sequence (e.g., having a length of about five to about 35 amino acids) capable of providing a spacer function such that the two sub-binding domains can interact to form a functional binding domain. In certain embodiments, an amino acid sequence that joins the VH and VL domains (also referred to herein as a “linker”) includes those belonging to the (GlyₙSer) family, such as (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5. In certain embodiments, the linker is GGGGSGGGGS GGGGS (SEQ ID NO:183) or GGGGSGGGGS GGGGSGGGGS (SEQ ID NO:108). In certain embodiments, these (GlyₙSer)-based linkers are used to link the VH and VL domains in a binding domain, but are not used to link a binding domain to an immunoglobulin heterodimerization domain or to an Fc region portion.

Exemplary binding domains specific for CD28 include a 2E12 scFv as set forth in SEQ ID NO:109, binding domains specific for CD79b include a P2C2 scFv as set forth in SEQ ID NO:185, binding domains specific for c-Met include a 5D5 scFv as set forth in SEQ ID NO:257, binding domains specific for RON include a 4C04 scFv as set forth in SEQ ID NO:261 and a 11H09 scFv as set forth in SEQ ID NO:265, and binding domains specific for CD3 include a humanized Cris7 scFv as set forth in SEQ ID NO:786.

The light chain amino acid sequence of the 4C04 scFv is set forth in SEQ ID NO:602, and its CDR1, CDR2, and CDR3 are set forth in SEQ ID NOS:604-606, respectively. The heavy chain amino acid sequence of the 4C04 scFv is set forth in SEQ ID NO:603, and its CDR1, CDR2, and CDR3 are set forth in SEQ ID NOS:607-609, respectively.

The light chain amino acid sequence of the 11H09 scFv is set forth in SEQ ID NO:610, and its CDR1, CDR2, and CDR3 are set forth in SEQ ID NOS:612-614, respectively. The heavy chain amino acid sequence of the 11H09 scFv is set forth in SEQ ID NO:611, and its CDR1, CDR2, and CDR3 are set forth in SEQ ID NOS:615-617, respectively.

Additional binding domains specific for c-Met comprise anti-c-Met light chain CDRs, anti-c-Met heavy chain CDRs, or both anti-c-Met light and heavy chain CDRs as shown in Tables 1 and 2. For example, a c-Met-specific binding domain may comprise: (a) light chain CDR1, CDR2 and CDR3 as set forth in SEQ ID NOS:296-298, respectively, (b) heavy chain CDR1, CDR2 and CDR3 as set forth in SEQ ID NOS:464-466, respectively, or (c) both light chain CDR1, CDR2 and CDR3 as set forth in SEQ ID NOS:296-298, respectively, and heavy chain CDR1, CDR2 and CDR3 as set forth in SEQ ID NOS:464-466, respectively,

TABLE 1
Anti-c-Met Light Chain Complementarity Determining Regions

| | | SEQ | | SEQ | | SEQ |
|---|---|---|---|---|---|---|
| Binding | | ID | | ID | | ID |
| Domain | CDR1 | NO. | CDR2 | NO. | CDR3 | NO. |
| TRU(H)-301 | QGDSLRNYHPS | 597 | GKNNRPS | 268 | NSRDSSGNLVF | 269 |
| TRU(H)-302 | SGDKLGDKYAS | 270 | QDRKRPS | 271 | QAWDSNTVV | 272 |
| TRU(H)-303 | SGSSSNIGSDYVH | 273 | RNNKRPS | 274 | AAWDDSLNGWV | 275 |
| TRU(H)-304 | SGDKLGDKYAS | 276 | EDNKRPS | 277 | QTWASGTVL | 278 |
| TRU(H)-305 | SGSSSNIGSNTVN | 279 | ANNQRPS | 280 | AVWDDSLNAWV | 281 |
| TRU(H)-306 | SGDKLENKYTS | 282 | EDIERPS | 283 | QAWDSNIAVV | 284 |
| TRU(H)-307 | SGGNSNIGSHYVY | 285 | RDNQRPS | 286 | AAWDDSLGGPV | 287 |
| TRU(H)-308 | SGSSSNIGRNAVN | 288 | NNNQRPS | 289 | AAWDDSLNGWV | 593 |
| TRU(H)-309 | GGNNIGDKSVH | 290 | EDKNRPA | 291 | QVWDSSTDHHV | 292 |
| TRU(H)-310 | GGNNIGTTSVQ | 293 | YGSDRPS | 294 | QTWVKGAGI | 295 |
| TRU(H)-311 | RASQSIRNYLN | 296 | AASSLQS | 297 | QQSYVTPLT | 298 |
| TRU(H)-312 | RASQSVNSLN | 299 | GISSLRR | 300 | QQSHSVPLT | 301 |
| TRU(H)-313 | RASQGIRNDLG | 302 | AASSLQS | 303 | LQHNSYPPT | 304 |
| TRU(H)-314 | RASQSVSSDLA | 305 | DAFKRAT | 306 | QQRSNWPLT | 307 |
| TRU(H)-315 | RASQSVSSYLA | 308 | DASNRAT | 309 | QQRSNWPLT | 310 |
| TRU(H)-316 | RSSQSLLYSNGYNYLD | 311 | LGSNRAS | 312 | MQALQSPLT | 313 |
| TRU(H)-317 | RASQSVSSSYLA | 314 | GASSRAT | 315 | QQRSI | 316 |
| TRU(H)-318 | RTSQYIRTNLA | 317 | DGSNRAT | 318 | QQRSNWPLT | 319 |
| TRU(H)-319 | RTSQQIMTYLN | 320 | VASRLQG | 321 | QQSFWTPLT | 322 |
| TRU(H)-320 | QASQDIDNYLN | 333 | DAYNLKA | 334 | QVFDDLSVT | 335 |
| TRU(H)-321 | RASQGIKNDLG | 336 | AASSLQS | 337 | QQSNSFPLT | 338 |
| TRU(H)-322 | QASHDINNYLN | 339 | DASNLQS | 340 | QQYDTLPVT | 341 |
| TRU(H)-323 | AGSSSNIGSNSVY | 342 | SNNKRPS | 343 | AAWDDSLRSVV | 344 |
| TRU(H)-324 | SGSSSTIGSNFVN | 345 | TNNQRPS | 346 | ATWDDNLLGPV | 347 |
| TRU(H)-325 | RASEGISSRLA | 348 | ATSSLQS | 349 | LQANTLPLT | 350 |
| TRU(H)-326 | RASLGVSNYLA | 351 | AASILQT | 352 | QHYQGYPYT | 353 |
| TRU(H)-327 | RASQSIDTYLN | 354 | AASKLED | 355 | QQSYSSPGIT | 356 |
| TRU(H)-328 | QASQDISDYLN | 357 | DASNLET | 358 | QQNDNLPFT | 359 |
| TRU(H)-329 | RASQSISSYLN | 360 | AASSLQS | 361 | QQSYSTPYT | 362 |
| TRU(H)-330 | RAGQAIRNNLG | 363 | AASSLQS | 364 | LQHNSFPYT | 365 |
| TRU(H)-331 | QASQDIINYLN | 366 | DASNLET | 367 | QQYDNLPYT | 368 |
| TRU(H)-332 | RATQSVRHNYLA | 369 | GAFFRAT | 370 | QQYGSSPVT | 371 |
| TRU(H)-333 | RASQSISSYLN | 372 | AASSLQS | 373 | QQSYSTSYT | 374 |
| TRU(H)-334 | RASQSVSSRYLA | 375 | AASSRAT | 376 | QQYGSSPPYT | 377 |
| TRU(H)-335 | RASQSVSFSLA | 378 | DTSNRVA | 379 | QHRSNWPG | 380 |
| TRU(H)-336 | QASQDIINYLN | 381 | DASNLET | 382 | QQYDNLPYT | 383 |
| TRU(H)-337 | QASQHISKYLN | 384 | DASNLET | 385 | QQYDNLPLT | 386 |
| TRU(H)-338 | RASQSIGSYLN | 387 | AATSLHT | 388 | QQYDNYPLT | 389 |
| TRU(H)-339 | RASQGIRNDLG | 390 | AASSLQS | 391 | LQHNSYPRT | 392 |
| TRU(H)-340 | RASQSVSSNLA | 393 | GASTRAT | 394 | QQYNNWPRT | 395 |
| TRU(H)-341 | RASQRIINYVS | 396 | GASTLQT | 397 | RQSYSSPLT | 398 |
| TRU(H)-342 | RASQTITTSLN | 399 | AASRLQN | 400 | QQSYNIPYT | 401 |
| TRU(H)-343 | RASQSIGSYLN | 402 | DASNLQS | 403 | QQSYRLFPT | 404 |
| TRU(H)-344 | QASQGIYNYVN | 405 | DASNLET | 406 | QQYDDVPIT | 407 |
| TRU(H)-345 | RASQGISSWLA | 408 | AASSLQS | 409 | QQANSFPIT | 410 |
| TRU(H)-346 | RSSQSIAKYLT | 411 | AASELQS | 412 | QQTYSFPHT | 413 |
| TRU(H)-347 | AGNNIGSKSVH | 414 | DDSDRPP | 415 | QVWDSDSDHYV | 416 |
| TRU(H)-348 | SGDRLGDKYAS | 417 | DDSERPS | 418 | QVWDSSIV | 419 |
| TRU(H)-349 | TGSTSDVGGYTYVS | 420 | DVSKRPS | 421 | CSYAGSYSYV | 422 |
| TRU(H)-350 | SGDKLGDKYAC | 423 | QDSKRPS | 424 | QAWDSSTYV | 425 |
| TRU(H)-351 | TGTSSDVGGHNYVS | 426 | DVSKRPS | 427 | CSYAGRYTYV | 428 |
| TRU(H)-352 | SGDRLEDKYTS | 429 | QDNKRPS | 430 | QAWDSSSAYV | 431 |
| TRU(H)-353 | GGNNIGSKSVH | 432 | FFDYDRPS | 433 | QVWDSRTDRYV | 434 |

TABLE 2
Anti-c-Met Heavy Chain Complementarity Determining Regions

| | | SEQ | | SEQ | | SEQ |
|---|---|---|---|---|---|---|
| Binding | | ID | | ID | | ID |
| Domain | CDR1 | NO. | CDR2 | NO. | CDR3 | NO. |
| TRU(H)- | IYDMH | 435 | WISPSGGRTLYADSVKG | 436 | TWDYYDSSGYFNDAFDI | 437 |
| 301 | | | | | | |
| TRU(H)- | AYNMA | 438 | SIVSSGGTTTYADSVKG | 439 | GGVGWLLDY | 440 |
| 302 | | | | | | |
| TRU(H)- | AYQMG | 441 | SISSSGGYTSYADSVKG | 442 | ESRYYFDY | 443 |
| 303 | | | | | | |
| TRU(H)- | EYPMI | 444 | GIGSSGGSTTYADSVKG | 445 | APLYSSTSYAFDI | 446 |
| 304 | | | | | | |
| TRU(H)- | FYWMI | 447 | GIGPSGGTTFYADSVKG | 448 | GGSYFDL | 594 |
| 305 | | | | | | |
| TRU(H)- | GYGMV | 449 | SISPSGGETLYADSVKG | 450 | GQMWPGVAFEM | 451 |
| 306 | | | | | | |
| TRU(H)- | LYFMT | 452 | SIGSSDGYTRYADSVKG | 453 | DLSWWPDAFDI | 454 |
| 307 | | | | | | |
| TRU(H)- | PYRME | 455 | WIYSSGGITNYADSVKG | 456 | | |
| 308 | | | | | | |
| TRU(H)- | VYDMV | 458 | SIGPSGGWTGYADSVKG | 459 | DSGGWEALYYYYYMDV | 460 |
| 309 | | | | | | |
| TRU(H)- | VYFMD | 461 | GIGPSGGVTSYADSVKG | 462 | GQLAQGHYYMDV | 463 |
| 310 | | | | | | |
| TRU(H)- | KYDML | 464 | YIYPSGGLTEYADSVKG | 465 | RAPRSLSFDI | 466 |
| 311 | | | | | | |
| TRU(H)- | RYMMA | 467 | SIYPSGGVTEYADSVKG | 468 | EGWYGYPT | 469 |
| 312 | | | | | | |
| TRU(H)- | RYMMG | 470 | VIVPSGGFTMYADSVKG | 471 | SSRLWSGYYPFDY | 472 |
| 313 | | | | | | |
| TRU(H)- | RYSMT | 473 | SIYSSGGETGYADSVKG | 474 | ERYNSFTS | 475 |
| 314 | | | | | | |
| TRU(H)- | SYVMV | 476 | VISPSGGVTFYADSVKG | 477 | DRRSNSLFDP | 478 |
| 315 | | | | | | |
| TRU(H)- | TYGMV | 479 | YIWPSGGLTWYADSVKG | 480 | SGYSYGRFDY | 481 |
| 316 | | | | | | |
| TRU(H)- | YYDMG | 482 | WISPSGGSTLYADSVKG | 483 | SGLYGSGSYAAFDV | 484 |
| 317 | | | | | | |
| TRU(H)- | YYHMV | 485 | YISPSGGDTHYADSVKG | 486 | GRYYGMDV | 487 |
| 318 | | | | | | |
| TRU(H)- | GYIMM | 488 | GIYPSGGGTDYADSVKG | 489 | ERPGYYDSTDDDYYYYP | 490 |
| 319 | | | | | MDV | |
| TRU(H)- | VYWME | 491 | SISSSGGLTSYADSVKG | 492 | DLVSNWPWGGY | 493 |
| 320 | | | | | | |
| TRU(H)- | HYKMG | 494 | SISSSGGDTAYADSVKG | 495 | DRAPYYYDSSGYYSDY | 496 |
| 321 | | | | | | |
| TRU(H)- | HYAMY | 497 | SISPSGGYTYYADSVKG | 498 | ESGTTNAFDI | 499 |
| 322 | | | | | | |
| TRU(H)- | NYHME | 500 | YISPSGGSTHYADSVKG | 501 | DGWTVPRD | 502 |
| 323 | | | | | | |
| TRU(H)- | RYWMA | 503 | SIVSSGGMTDYADSVKG | 504 | HRGDSGLDY | 505 |
| 324 | | | | | | |
| TRU(H)- | HYPML | 506 | GISSSGGWTDYADSVKG | 507 | DRALGMDV | 508 |
| 325 | | | | | | |
| TRU(H)- | LYSMV | 509 | RIRPSGGQTMYADSVKG | 510 | GYAFDI | 511 |
| 326 | | | | | | |
| TRU(H)- | FYDML | 512 | SIWSSGGQTGYADSVKG | 513 | EKASDLSGSYSEALDY | 514 |
| 327 | | | | | | |
| TRU(H)- | NYHMN | 515 | YIYPSGGVTYYADSVKG | 516 | GIAAAGNYYYYYGMDV | 517 |
| 328 | | | | | | |
| TRU(H)- | KYGMV | 518 | SISSSGGNTAYADSVKG | 519 | GELERRRRNYYGMDV | 520 |
| 329 | | | | | | |
| TRU(H)- | NYAMT | 521 | SIYSSGGDTAYADYVKG | 522 | EYYTGWNFDY | 523 |
| 330 | | | | | | |
| TRU(H)- | QYDMV | 524 | YIYSSGGHTLYVDSVKG | 525 | IRSSGYYHEVLDY | 526 |
| 331 | | | | | | |
| TRU(H)- | TYMMY | 527 | VIGPSGGATGYADSVKG | 528 | IRKAFGYGSGSLDY | 529 |
| 332 | | | | | | |
| TRU(H)- | YYDMQ | 530 | YIGPSGGDTDYADSVKG | 531 | SSYYYDSSGYYHEAFDI | 532 |
| 333 | | | | | | |
| TRU(H)- | YYMMR | 533 | YIGPSGGATTYADSVKG | 534 | GRSVKYYYDSSGYLLFD | 535 |
| 334 | | | | | Y | |
| TRU(H)- | HYSMY | 536 | GIYSSGGPTIYADSVKG | 537 | LQIEMATIGHFDY | 538 |
| 335 | | | | | | |
| TRU(H)- | QYDMV | 539 | YIYSSGGHTLYVDSVKG | 540 | IRSSGYYHEVLDY | 541 |
| 336 | | | | | | |
| TRU(H)- | HYWMM | 542 | SIVPSGGDTYYADSVKG | 543 | DPVMTPVDY | 544 |
| 337 | | | | | | |
| TRU(H)- | PYFMN | 545 | SIYPSGGITKYADSVKG | 546 | ETYYYGSGSYAFDI | 547 |
| 338 | | | | | | |
| TRU(H)- | QYYMY | 548 | RISPSGGMTSYADSVKG | 549 | HKYGGPDF | 550 |
| 339 | | | | | | |
| TRU(H)- | RYQMN | 551 | SIRSSGGVTKYADSVKG | 552 | GRGLSS | 553 |
| 340 | | | | | | |
| TRU(H)- | LYTMA | 554 | YISPSGGFTGYADSVKG | 555 | WGDP | 556 |
| 341 | | | | | | |
| TRU(H)- | DYFMG | 557 | RISSSGGHTMYADSVKG | 558 | EEDYYDSSGYYPPAFDI | 559 |
| 342 | | | | | | |
| TRU(H)- | IYWMY | 560 | GIGPSGGYTSYADSVKG | 561 | GNGGFDS | 562 |
| 343 | | | | | | |
| TRU(H)- | PYHMS | 563 | SIYPSGGFTAYADSVKG | 564 | ESAYYYDSSPPAFDI | 565 |
| 344 | | | | | | |
| TRU(H)- | TYAMY | 566 | SIYSSGGATWYADSVKG | 567 | STFDYFDY | 568 |
| 345 | | | | | | |
| TRU(H)- | KYRMM | 569 | YISSSGGATIYADSVKG | 570 | HGPQIAAWYFDL | 571 |
| 346 | | | | | | |
| TRU(H)- | PYSMD | 572 | GISSSGGRTVYADSVKG | 573 | GPYYDFWSGYYIDRGPL | 574 |
| 347 | | | | | DY | |
| TRU(H)- | WYMMA | 575 | WISSSGGFTPYADSVKG | 576 | GQQWPGVAFDI | 577 |
| 348 | | | | | | |
| TRU(H)- | FYPMM | 578 | YIGPSGGNNADSVKG | 579 | GLWFGGRLDY | 580 |
| 349 | | | | | | |
| TRU(H)- | HYWMK | 581 | GISSSGGQTDYADSVKG | 582 | SPRLRFLEWPRNYYGMD | 583 |
| 350 | | | | | V | |
| TRU(H)- | LYMMV | 584 | YIGPSGGAYADSVKG | 585 | SVRGLTFDY | 586 |
| 351 | | | | | | |
| TRU(H)- | PYEMG | 587 | RISPSGGMTLYADSVKG | 588 | MGRGGWWAFDAFDI | 589 |
| 352 | | | | | | |
| TRU(H)- | WYKMV | 590 | GIYPSGGTTHYADSVKG | 591 | GGGDFWSGYYPFDY | 592 |
| 353 | | | | | | |

A target molecule, which is specifically bound by a binding domain contained in a polypeptide heterodimer of the present disclosure, may be found on or in association with a cell of interest (“target cell”). Exemplary target cells include a cancer cells, a cell associated with an autoimmune disease or disorder or with an inflammatory disease or disorder, and an infectious cell (e.g., an infectious bacterium). A cell of an infectious organism, such as a mammalian parasite, is also contemplated as a target cell.

In certain embodiments, binding domains of polypeptide heterodimers of the present disclosure recognize a target selected from a tumor antigen, a B-cell target, a TNF receptor superfamily member, a Hedgehog family member, a receptor tyrosine kinase, a proteoglycan-related molecule, a TGF-β superfamily member, a Wnt-related molecule, a T-cell target, a Dendritic cell target, an NK cell target, a monocyte/macrophage cell target, or an angiogenesis target. Specific examples of such targets may be found, for example, in PCT Publication No. WO 2007/146968, which targets are incorporated herein by reference. In further embodiments, the binding domains of polypeptide heterodimers of the present disclosure bind a receptor protein, such as peripheral membrane receptor proteins or transmembrane receptor proteins.

In certain embodiments, a polypeptide heterodimer of the present disclosure specifically binds a target such as c-Met, RON, CD3, CEACAM6, EGFR, ErbB3, ErbB4, EphA2, IGF1R, GHRHR, GHR, FLT1, KDR, FLT4, CD44v6, CD151, GITR, BTLA, TGFBR2, TGFBR1, IL6R, gp130, TNFR1, TNFR2, PD1, PD-L1, PD-L2, HVEM, RANK, TNFRSF4, CD40, CD137, TWEAK-R, LTβR, LIFRβ, LRP5, OSMRβ, TCRα, TCRβ, CD19, CD28, CD80, CD81, CD86, TLR7, TLR9, PTCH1, Robo1, Frizzled, OX40 (also referred to as CD134), and CD79b.

A binding domain may be located either amino terminal or carboxyl terminal to the Fc region portion of a single chain polypeptide of the present disclosure. In certain embodiments, the binding domain is located at the amino terminus of a single chain polypeptide. In certain other embodiments, the binding domain is located at the carboxyl terminus of a single chain polypeptide.

A single chain polypeptide comprising a binding domain may comprise a CH1 region as an immunoglobulin heterodimerization domain. Alternatively, a single chain polypeptide that comprises a binding domain may comprise a CL domain as an immunoglobulin heterodimerization domain.

### Heterodimerization Domains

As indicated above, a polypeptide heterodimer of the present disclosure comprises an immunoglobulin heterodimerization domain in each polypeptide chain. The immunoglobulin heterodimerization domain in a first chain of a polypeptide heterodimer is different from the immunoglobulin heterodimerization domain in a second chain of the heterodimer so that the immunoglobulin heterodimerization domains may be differentially modified to facilitate heterodimerization of the first and second chains and to minimize first chain homodimerization or second chain homodimerization. As shown in the examples, immunoglobulin heterodimerization domains provided herein allow for efficient heterodimerization between different polypeptides and can facilitate purification of the resulting polypeptide heterodimers.

As provided herein, immunoglobulin heterodimerization domains useful for promoting heterodimerization of two different single chain polypeptides (e.g., one short and one long) according to the present disclosure include immunoglobulin CH1 and CL domains, for instance, human CH1 and CL domains. In certain embodiments, an immunoglobulin heterodimerization domain is a wild type CH1 region, such as a wild type IgG1, IgG2, IgG3, IgG4, IgA1, IgA2 IgD, IgE, or IgM CH1 region. In further embodiments, an immunoglobulin heterodimerization domain is a wild type human IgG1, IgG2, IgG3, IgG4, IgA1, IgA2, IgD, IgE, or IgM CH1 region as set forth in SEQ ID NOS:114, 186-192 and 194, respectively. In certain embodiments, an immunoglobulin heterodimerization domain is a wild type human IgG1 CH1 region as set forth in SEQ ID NO:114.

In further embodiments, an immunoglobulin heterodimerization domain is an altered immunoglobulin CH1 region, such as an altered IgG1, IgG2, IgG3, IgG4, IgA1, IgA2 IgD, IgE, or IgM CH1 region. In certain embodiments, an immunoglobulin heterodimerization domain is an altered human IgG1, IgG2, IgG3, IgG4, IgA1, IgA2, IgD, IgE, or IgM CH1 region. In still further embodiments, a cysteine residue of a wild type CH1 region (e.g., a human CH1) involved in forming a disulfide bond with a wild type immunoglobulin CL domain (e.g., a human CL) is deleted or substituted in the altered immunoglobulin CH1 region such that a disulfide bond is not formed between the altered CH1 region and the wild type CL domain.

In certain embodiments, an immunoglobulin heterodimerization domain is a wild type CL domain, such as a wild type Cδ domain or a wild type Cλ domain. In particular embodiments, an immunoglobulin heterodimerization domain is a wild type human Cκ or human Cλ domain as set forth in SEQ ID NOS:112 and 113, respectively. In further embodiments, an immunoglobulin heterodimerization domain is an altered immunoglobulin CL domain, such as an altered Cκ or Cλ domain, for instance, an altered human Cκ or human Cλ domain.

In certain embodiments, a cysteine residue of a wild type CL domain (e.g., a human CL) involved in forming a disulfide bond with a wild type immunoglobulin CH1 region (e.g., a human CH1) is deleted or substituted in the altered immunoglobulin CL domain. Such altered CL domains may further comprise an amino acid deletion at their amino termini. An exemplary Cκ domain is set forth in SEQ ID NO:141, in which the first arginine and the last cysteine of the wild type human Ck domain are both deleted. In certain embodiments, only the last cysteine of the wild type human Ck domain is deleted in the altered Ck domain because the first arginine deleted from the wild type human Ck domain may be provided by a linker that has an arginine at its carboxyl terminus and links the amino terminus of the altered Ck domain with another domain (e.g., an Fc region portion). An exemplary Cλ domain is set forth in SEQ ID NO:140, in which the first arginine of a wild type human Cλ domain is deleted and the cysteine involved in forming a disulfide bond with a cysteine in a CH1 region is substituted by a serine.

In further embodiments, an immunoglobulin heterodimerization domain is an altered Cκ domain that contains one or more amino acid substitutions, as compared to a wild type Cκ domain, at positions that may be involved in forming the interchain-hydrogen bond network at a Cκ-Cκ interface. For example, in certain embodiments, an immunoglobulin heterodimerization domain is an altered human Cκ domain having one or more amino acids at positions N29, N30, Q52, V55, T56, S68 or T70 that are substituted with a different amino acid. The numbering of the amino acids is based on their positions in the altered human Cκ sequence as set forth in SEQ ID NO:141. In certain embodiments, an immunoglobulin heterodimerization domain is an altered human Cκ domain having one, two, three or four amino acid substitutions at positions N29, N30, V55, or T70. The amino acid used as a substitute at the above-noted positions may be an alanine, or an amino acid residue with a bulk side chain moiety such as arginine, tryptophan, tyrosine, glutamate, glutamine, or lysine. Additional amino acid residues that may be used to substitute amino acid residues of the wild type human Ck sequence at the above noted positions (e.g., N30) include aspartate, methionine, serine and phenyalanine. Exemplary altered human Cκ domains are set forth in SEQ ID NOS:142-178. Examples of altered human Cκ domains are those that facilitate heterodimerization with a CH1 region, but minimize homodimerization with another Cκ domain. Representative altered human Cκ domains are set forth in SEQ ID NOS:160 (N29W V55A T70A), 161 (N29Y V55A T70A), 202 (T70E N29A N30A V55A), 167 (N30R V55A T70A), 168 (N30K V55A T70A), 170 (N30E V55A T70A), 172 (V55R N29A N30A), 175 (N29W N30Y V55A T70E), 176 (N29Y N30Y V55A T70E), 177 (N30E V55A T70E), 178 (N30Y V55A T70E), 770 (N30D V55A T70E), 771 (N30M V55A T70E), 772 (N305 V55A T70E), and 773 (N30F V55A T70E).

In certain embodiments, in addition to or alternative to the mutations in Ck domains described herein, both the immunoglobulin heterodimerization domains (i.e., immunoglobulin CH1 and CL domains) of a polypeptide heterodimer have mutations so that the resulting heterodimerization domains form salt bridges (i.e., ionic interactions) between the amino acid residues at the mutated sites. For example, the heterodimerization domains of a polypeptide heterodimer may be a mutated CH1 domain in combination with a mutated Ck domain. In the mutated CH1 domain, valine at position 68 (V68) of the wild type human CH1 domain is substituted by an amino acid residue having a negative charge (e.g., asprartate or glutamate), whereas leucine at position 29 (L29) of a mutated human Ck domain in which the first arginine and the last cysteine have been deleted is substituted by an amino acid residue having a positive charge (e.g., lysine, arginine or histidine). The charge-charge interaction between the amino acid residue having a negative charge of the resulting mutated CH 1 domain and the amino acid residue having a positive charge of the resulting mutated Ck domain forms a salt bridge, which stabilizes the heterodimeric interface between the mutated CH1 and Ck domains. Alternatively, V68 of the wild type CH1 may be substituted by an amino acid residue having a positive charge, whereas L29 of a mutated human Ck domain in which the first arginine and the last cysteine have been deleted may be substituted by an amino acid residue having a negative charge. Exemplary mutated CH1 sequences in which V68 is substituted by an amino acid with either a negative or positive charge are set forth in SEQ ID NOS:784 and 785. Exemplary mutated Ck sequences in which L29 is substituted by an amino acid with either a negative or positive charge are set forth in SEQ ID NOS:782 and 783.

Positions other than V68 of human CH1 domain and L29 of human Ck domain may be substituted with amino acids having opposite charges to produce ionic interactions between the amino acids in addition or alternative to the mutations in V68 of CH1 domain and L29 of Ck domain. Such positions can be identified by any suitable method, including random mutagenesis, analysis of the crystal structure of the CH1-Ck pair to identify amino acid residues at the CH1-Ck interface, and further identifying suitable positions among the amino acid residues at the CH1-Ck interface using a set of criteria (e.g., propensity to engage in ionic interactions, proximity to a potential partner residue, etc.).

In certain embodiments, polypeptide heterodimers of the present disclosure contain only one pair of immunoglobulin heterodimerization domains. For example, a first chain of a polypeptide heterodimer may comprise a CH1 region as an immunoglobulin heterodimerization domain, while a second chain may comprise a CL domain (e.g., a Cκ or Cλ) as an immunoglobulin heterodimerization domain. Alternatively, a first chain may comprise a CL region (e.g., a Cκ or Cλ) as an immunoglobulin heterodimerization domain, while a second chain may comprise a CH1 region as an immunoglobulin heterodimerization domain. As set forth herein, the immunoglobulin heterodimerization domains of the first and second chains are capable of associating to form a polypeptide heterodimer of this disclosure.

In certain other embodiments, polypeptide heterodimers of the present disclosure may have two pairs of immunoglobulin heterodimerization domains. For example, a first chain of a polypeptide heterodimer may comprise two CH1 regions, while a second chain may have two CL domains that associate with the two CH1 regions in the first chain. Alternatively, a first chain may comprise two CL domains, while a second chain may have two CH1 regions that associate with the two CL domains in the first chain. In certain embodiments, a first chain polypeptide comprises a CH1 region and a CL domain, while a second chain polypeptide comprises a CL domain and a CH1 region that associate with the CH1 region and the CL domain, respectively, of the first chain polypeptide.

In the embodiments where a polypeptide heterodimer comprises only one heterodimerization pair (i.e., one immunoglobulin heterodimerization domain in each chain), the immunoglobulin heterodimerization domain of each chain may be located amino terminal to the Fc region portion of that chain. Alternatively, the immunoglobulin heterodimerization domain in each chain may be located carboxyl terminal to the Fc region portion of that chain.

In the embodiments where a polypeptide heterodimer comprises two heterodimerization pairs (i.e., two immunoglobulin heterodimerization domains in each chain), both immunoglobulin heterodimerization domains in each chain may be located amino terminal to the Fc region portion of that chain. Alternatively, both immunoglobulin heterodimerization domains in each chain may be located carboxyl terminal to the Fc region portion of that chain. In further embodiments, one immunoglobulin heterodimerization domain in each chain may be located amino terminal to the Fc region portion of that chain, while the other immunoglobulin heterodimerization domain of each chain may be located carboxyl terminal to the Fc region portion of that chain. In other words, in those embodiments, the Fc region portion is interposed between the two immunoglobulin heterodimerization domains of each chain.

### Portion of Fc Region

As indicated herein, polypeptide heterodimers of the present disclosure comprise an Fc region constant domain portion (also referred to as an Fc region portion) in each polypeptide chain. The inclusion of an Fc region portion slows clearance of the heterodimers from circulation after administration to a subject. By mutation or other alteration, the Fc region portion further enables relatively easy modulation of heterodimer polypeptide effector function (e.g., ADCC, ADCP, CDC), which can either be increased or decreased depending on the disease being treated, as is known in the art and described herein.

An Fc region portion present in single chain polypeptides that form part of the polypeptide heterodimers of the present disclosure may comprise a CH2 domain, a CH3 domain, a CH4 domain or any combination thereof. For example, an Fc region portion may comprise a CH2 domain, a CH3 domain, both CH2 and CH3 domains, both CH3 and CH4 domains, two CH3 domains, a CH4 domain, or two CH4 domains. In certain embodiments, the Fc region portion is an IgG CH2CH3, for instance, a human CH2CH3.

A CH2 domain that may form an Fc region portion of a single chain polypeptide of a heterodimer of the present disclosure may be a wild type immunoglobulin CH2 domain or an altered immunoglobulin CH2 domain thereof from certain immunoglobulin classes or subclasses (e.g., IgG1, IgG2, IgG3, IgG4, IgA1, IgA2, or IgD) and from various species (including human, mouse, rat, and other mammals).

In certain embodiments, a CH2 domain is a wild type human immunoglobulin CH2 domain, such as wild type CH2 domains of human IgG1, IgG2, IgG3, IgG4, IgA1, IgA2, or IgD, as set forth in SEQ ID NOS:115, 199-201 and 195-197, respectively. In certain embodiments, the CH2 domain is a wild type human IgG1 CH2 domain as set forth in SEQ ID NO:115.

In certain embodiments, a CH2 domain is an altered immunoglobulin CH2 region (e.g., an altered human IgG1 CH2 domain) that comprises an amino acid substitution at the asparagine of position 297 (e.g., asparagine to alanine) Such an amino acid substitution reduces or eliminates glycosylation at this site and abrogates efficient Fc binding to FcγR and C1q.

In certain embodiments, a CH2 domain is an altered immunoglobulin CH2 region (e.g., an altered human IgG1 CH2 domain) that comprises at least one substitution or deletion at positions 234 to 238. For example, an immunoglobulin CH2 region can comprise a substitution at position 234, 235, 236, 237 or 238, positions 234 and 235, positions 234 and 236, positions 234 and 237, positions 234 and 238, positions 234-236, positions 234, 235 and 237, positions 234, 236 and 238, positions 234, 235, 237, and 238, positions 236-238, or any other combination of two, three, four, or five amino acids at positions 234-238. In addition or alternatively, an altered CH2 region may comprise one or more (e.g., about two, three, four or five) amino acid deletions at positions 234-238, for instance a deletion at one of position 236 or position 237 while the other position is substituted. The above-noted mutation(s) decrease or eliminate the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In certain other embodiments, a CH2 domain is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. The above-noted mutation(s) decrease or eliminate the complement-dependent cytotoxicity (CDC) of a polypeptide heterodimer that comprises the altered CH2 domain.

In certain other embodiments, in addition to the amino acid substitution at position 297, an altered CH2 region (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region may comprise one or more (e.g., two, three, four or five) amino acid deletions at positions 234-238, such as at position 236 or position 237. The additional mutation(s) decreases or eliminates the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., about 2, 3, 4,or 5) amino acid substitutions at positions 234-238, a mutated CH2 region (e.g., an altered human IgG1 CH2 domain) in a fusion protein of the present disclosure may contain one or more (e.g., 2, 3, 4, 5, or 6) additional amino acid substitutions (e.g., substituted with alanine) at one or more positions involved in complement fixation (e.g., at positions 1253, H310, E318, K320, K322, or P331). Mutated immunoglobulin CH2 regions can include human IgG1, IgG2, IgG4 and mouse IgG2a CH2 regions with alanine substitutions at positions 234, 235, 237 (if present), 318, 320 and 322.

In still further embodiments, in addition to the amino acid substitution at position 297 and the additional deletion(s) or substitution(s) at positions 234-238, an altered CH2 region (e.g., an altered human IgG1 CH2 domain) can further comprise one or more (e.g., two, three, four, five, or six) additional substitutions at positions 253, 310, 318, 320, 322, and 331. For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. For instance, the amino acids at the above-noted positions are substituted by alanine or serine.

In certain embodiments, an immunoglobulin CH2 region polypeptide comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary altered immunoglobulin CH2 regions with amino acid substitutions at the asparagine of position 297 include: human IgG1 CH2 region with alanine substitutions at L234, L235, G237 and N297 and a deletion at G236, human IgG2 CH2 region with alanine substitutions at V234, G236, and N297, human IgG4 CH2 region with alanine substitutions at F234, L235, G237 and N297 and a deletion of G236, human IgG4 CH2 region with alanine substitutions at F234 and N297, human IgG4 CH2 region with alanine substitutions at L235 and N297, human IgG4 CH2 region with alanine substitutions at G236 and N297, and human IgG4 CH2 region with alanine substitutions at G237 and N297.

In certain embodiments, in addition to the amino acid substitutions described above, an altered CH2 region may contain one or more additional amino acid substitutions at one or more positions other than the above-noted positions. Such amino acid substitutions may be conservative or non-conservative amino acid substitutions. For example, in certain embodiments, P233 may be changed to E233 in an altered IgG2 CH2 region. In addition or alternatively, in certain embodiments, the altered CH2 region may contain one or more amino acid insertions, deletions, or both. The insertion(s), deletion(s) or substitution(s) may anywhere in an immunoglobulin CH2 region, such as at the N- or C-terminus of a wild type immunoglobulin CH2 region resulting from linking the CH2 region with another region (e.g., a binding domain or an immunoglobulin heterodimerization domain) via a hinge.

In certain embodiments, an altered CH2 region in a polypeptide heterodimer of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a polypeptide heterodimer of the present disclosure may be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure may be derived from a CH2 region of human IgG1, IgG2 or IgG4, or mouse IgG2a (e.g., IGHG2c).

In certain embodiments, an altered CH2 domain is a human IgG1 CH2 domain with alanine substitutions at positions 235, 318, 320, and 322 (i.e., a human IgG1 CH2 domain with L235A, E318A, K320A and K322A substitutions) (SEQ ID NO:595), and optionally an N297 mutation (e.g., to alanine) In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO:596), and optionally an N297 mutation (e.g., to alanine)

In certain embodiments, an altered CH2 domain is an altered human IgG1 CH2 domain with mutations known in the art that enhance immunological activities such as ADCC, ADCP, CDC, complement fixation, Fc receptor binding, or any combination thereof

The CH3 domain that may form an Fc region portion of a single chain polypeptide of a heterodimer of the present disclosure may be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain is a wild type human immunoglobulin CH3 domain, such as wild type CH3 domains of human IgG1, IgG2, IgG3, IgG4, IgA1, IgA2, IgD, IgE, or IgM as set forth in SEQ ID NOS:116, 208-210, 204-207, and 212, respectively. In certain embodiments, the CH3 domain is a wild type human IgG1 CH3 domain as set forth in SEQ ID NO:116. In certain embodiments, a CH3 domain is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain may be a human IgG1 CH3 domain with one or two mutations at positions H433 and N434 (positions are numbered according to EU numbering). The mutations in such positions may be involved in complement fixation. In certain other embodiments, an altered CH3 domain may be a human IgG1 CH3 domain but with one or two amino acid substitutions at position F405 or Y407. The amino acids at such positions are involved in interacting with another CH3 domain. In certain embodiments, an altered CH3 domain may be an altered human IgG1 CH3 domain with its last lysine deleted. The sequence of this altered CH3 doamin is set forth in SEQ ID NO:794.

In certain embodiments, a polypeptide heterodimer comprises a CH3 pair that comprises so called “knobs-into-holes” mutations (see, Marvin and Zhu, Acta Pharmacologica Sinica 26:649-58, 2005; Ridgway et al., Protein Engineering 9:617-21, 1966). More specifically, mutations may be introduced into each of the two CH3 domains so that the steric complementarity required for CH3/CH3 association obligates these two CH3 domains to pair with each other. For example, a CH3 domain in one single chain polypeptide of a polypeptide heterodimer may contain a T366W mutation (a “knob” mutation, which substitutes a small amino acid with a larger one), and a CH3 domain in the other single chain polypeptide of the polypeptide heterodimer may contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that may form an Fc region portion of a single chain polypeptide of a heterodimer of the present disclosure may be a wild type immunoglobulin CH4 domain or an altered immunoglobulin CH4 domain thereof from IgE or IgM molecules. In certain embodiments, the CH4 domain is a wild type human immunoglobulin CH4 domain, such as wild type CH4 domains of human IgE and IgM molecules as set forth in SEQ ID NOS:213 and 214, respectively.

In certain embodiments, a CH4 domain is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an Fc region constant domain portion in heterodimers of the present disclosure comprises a combination of CH2, CH3 or CH4 domains (i.e., more than one constant sub-domain selected from CH2, CH3 and CH4). For example, the Fc region portion may comprise CH2 and CH3 domains or CH3 and CH4 domains. In certain other embodiments, the Fc region portion may comprise two CH3 domains and no CH2 or CH4 domains (i.e., only two or more CH3). The multiple constant sub-domains that form an Fc region portion may be based on or derived from the same immunoglobulin molecule, or the same class or subclass immunoglobulin molecules.

Alternatively, the multiple constant sub-domains may be based on or derived from different immunoglobulin molecules, or different classes or subclasses immunoglobulin molecules. For example, in certain embodiments, an Fc region portion comprises both human IgM CH3 domain and human IgG1 CH3 domain. The multiple constant sub-domains that form an Fc region portion may be directly linked together or may be linked to each other via one or more (e.g., 2-8) amino acids.

Exemplary Fc region portions are set forth in SEQ ID NOS:795 and 882-889.

In certain embodiments, an Fc constant domain region portion comprises a wild type human IgG1 CH2 domain and a wild type human IgG1 CH3 domain. In certain other embodiments, an Fc region portion comprises an altered human IgG1 CH2 domain (e.g., having an amino acid mutation at N297 or having at least one additional amino acid mutation at positions 234-238 or having amino acid mutations at positions 234, 235, 237, 318, 320 and 322) and a wild type human CH3 domain, so that the Fc region portion of a heterodimer of this disclosure does not promote immunological activities, such as ADCC, ADCP, CDC, Fc receptor binding, or any combination thereof. In other embodiments, an altered human IgG1 CH2 domain can have mutations known in the art to enhance immunological activities, such as ADCC, ADCP, CDC, Fc receptor binding, or any combination thereof. In certain other embodiments, an Fc region portion comprises a wild type human IgM CH3 domain and a wild type human IgM CH4 domain, or a wild type human IgE CH3 domain and a wild type human IgE CH4 domain.

In certain embodiments, the Fc region portions of both single chain polypeptides of a polypeptide heterodimer are identical to each other. In certain other embodiments, the Fc region portion of one single chain polypeptide of a polypeptide heterodimer is different from the Fc region portion of the other single chain polypeptide of the heterodimer. For example, one Fc region portion may contain a CH3 domain with a “knob” mutation, whereas the other Fc region portion may contain a CH3 domain with a “hole” mutation.

### Hinge

A hinge region contained in a single chain polypeptide of a polypeptide heterodimer according to the present disclosure may be located (a) immediately amino terminal to an Fc region portion (e.g., depending on the isotype, amino terminal to a CH2 domain wherein the Fc region portion is a CH2CH3, or amino terminal to a CH3 domain wherein the Fc region portion is a CH3CH4), (b) interposed between and connecting a binding domain (e.g., scFv) and an immunoglobulin heterodimerization domain, (c) interposed between and connecting an immunoglobulin heterodimerization domain and an Fc region portion (e.g., wherein the Fc region portion is a CH2CH3 or a CH3CH4, depending on the isotype or isotypes), (d) interposed between and connecting an Fc region portion and a binding domain, (e) at the amino terminus of the single chain polypeptide, or (f) at the carboxyl terminus of the single chain polypeptide. The single chain polypeptide comprising a hinge region as described herein will be capable of associating with a different single chain fusion polypeptide to form a polypeptide heterodimer provided herein, and the polypeptide heterodimer formed will contain a binding domain that retains its target specificity or its specific target binding affinity.

In certain embodiments, a hinge present in a single chain polypeptide that forms a polypeptide heterodimer with another single chain polypeptide may be an immunoglobulin hinge region, such as a wild type immunoglobulin hinge region or an altered immunoglobulin hinge region thereof

In certain embodiments, a hinge is a wild type human immunoglobulin hinge region (e.g., human immunoglobulin hinge regions as set forth in SEQ ID NOS:215-221). In certain other embodiments, one or more amino acid residues may be added at the amino- or carboxy-terminus of a wild type immunoglobulin hinge region as part of a fusion protein construct design. For example, additional junction amino acid residues at the hinge amino-terminus can be “RT,” “RSS,” “TG,” or “T”, or at the hinge carboxy-terminus can be “SG”, or a hinge deletion can be combined with an addition, such as ΔP with “SG” added at the carboxyl terminus.

In certain embodiments, a hinge is an altered immunoglobulin hinge in which one or more cysteine residues in a wild type immunoglobulin hinge region is substituted with one or more other amino acid residues (e.g., serine or alanine) For example, a hinge may be an altered immunoglobulin hinge based on or derived from a wild type human IgG1 hinge as set forth in SEQ ID NO:218, which from amino terminus to carboxyl terminus comprises the upper hinge region (EPKSCDKTHT, SEQ ID NO:227) and the core hinge region (CPPCP, SEQ ID NO:228). Exemplary altered immunoglobulin hinges include an immunoglobulin human IgG1 hinge region having one, two or three cysteine residues found in a wild type human IgG1 hinge substituted by one, two or three different amino acid residues (e.g., serine or alanine) An altered immunoglobulin hinge may additionally have a proline substituted with another amino acid (e.g., serine or alanine) For example, the above-described altered human IgG1 hinge may additionally have a proline located carboxyl terminal to the three cysteines of wild type human IgG1 hinge region substituted by another amino acid residue (e.g., serine, alanine) In one embodiment, the prolines of the core hinge region are not substituted. Exemplary altered immunoglobulin hinges are set forth in SEQ ID NOS: 229-240, 255, 664-677, and 748-759. An example of an altered IgG1 hinge is an altered human IgG1 hinge in which the first cysteine is substituted by serine. The sequence of this altered IgG1 hinge is set forth in SEQ ID NO:664, and is referred to as the “ human IgG1 SCC-P hinge” or “SCC-P hinge.” In certain embodiments, one or more amino acid residues (e.g., “RT,” “RSS,” or “T”) may be added at the amino-or carboxy-terminus of a mutated immunoglobulin hinge region as part of a fusion protein construct design.

In certain embodiments, a hinge polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a wild type immunoglobulin hinge region, such as a wild type human IgG1 hinge, a wild type human IgG2 hinge, or a wild type human IgG4 hinge.

In further embodiments, a hinge present in a single chain polypeptide that forms a polypeptide heterodimer with another single chain polypeptide may be a hinge that is not based on or derived from an immunoglobulin hinge (i.e., not a wild type immunoglobulin hinge or an altered immunoglobulin hinge). These types of non-immunoglobulin based hinges can be used on or near the carboxyl end (e.g., located carboxyl terminal to Fc region portions) of the single chain polypeptides that form the polypeptide heterodimers. Examples for such hinges include peptides of about five to about 150 amino acids of the interdomain or stalk region of type II C-lectins or CD molecules, for instance, peptides of about eight to 25 amino acids and peptides of about seven to 18 amino acids, and derivatives thereof

The “interdomain or stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the transmembrane domain. For example, in the human CD94 molecule (GenBank Accession No. AAC50291.1, PRI Nov. 30, 1995), the extracellular domain corresponds to amino acid residues 34-179, whereas the CTLD corresponds to amino acid residues 61-176. Accordingly, the interdomain or stalk region of the human CD94 molecule includes amino acid residues 34-60, which is found between the membrane and the CTLD (see Boyington et al., Immunity 10:75, 1999; for descriptions of other stalk regions, see also Beavil et al., Proc. Nat'l. Acad. Sci. USA 89:753, 1992; and Figdor et al., Nature Rev. Immunol. 2:77, 2002). These type II C-lectin or CD molecules may also have from six to 10 junction amino acids between the stalk region and the transmembrane region or the CTLD. In another example, the 233 amino acid human NKG2A protein (GenBank Accession No. P26715.1, PRI Jun. 15, 2010) has a transmembrane domain ranging from amino acids 71-93 and an extracellular domain ranging from amino acids 94-233. The CTLD is comprised of amino acids 119-231, and the stalk region comprises amino acids 99-116, which is flanked by junctions of five and two amino acids. Other type II C-lectin or CD molecules, as well as their extracellular ligand-bind domains, interdoamin or stalking regions, and CTLDs are known in the art (see, e.g., GenBank Accession Nos. NP_{—}001993.2; AAH07037.1, PRI Jul. 15, 2006; NP_{—}001773.1, PRI Jun. 20, 1010; AAL65234.1, PRI Jan. 17, 2002, and CAA04925.1, PRI Nov. 14, 2006, for the sequences of human CD23, CD69, CD72, NKG2A and NKG2D and their descriptions, respectively).

A “derivative” of an interdomain or stalk region, or fragment thereof, of a type II C-lectin or CD molecule includes an about an eight to about 150 amino acid sequence in which one, two, or three amino acids of the stalk region of a wild type type II C-lectin or CD molecule have a deletion, insertion, substitution, or any combination thereof. For instance, a derivative can comprise one or more amino acid substitutions and/or an amino acid deletion. In certain embodiments, a derivative of an interdomain or stalk region is more resistant to proteolytic cleavage as compared to the wild-type interdomain or stalk region sequence, such as those derived from about eight to about 20 amino acids of NKG2A, NKG2D, CD23, CD64, CD72, or CD94.

In certain embodiments, interdomain or stalk region hinges may comprise about seven to 18 amino acids and can form an a-helical coiled coil structure. In certain embodiments, interdomain or stalk region hinges contain about 0, 1, 2, 3, or 4 cysteines. Exemplary interdomain or stalk region hinges are peptide fragments of the interdomain or stalk regions, such as ten to 150 amino acid fragments from the stalk regions of CD69, CD72, CD94, NKG2A and NKG2D, as set forth in SEQ ID NOS:125, 241-244, 601, and 716. Additional exemplary stalk region or interdomain hinges include those as set forth in SEQ ID NOS:734-737, 742-747, 799-823, and 825.

Alternative hinges that can be used in single chain polypeptides of polypeptide heterodimers are from portions of cell surface receptors (interdomain regions) that connect immunoglobulin V-like or immunoglobulin C-like domains. Regions between Ig V-like domains where the cell surface receptor contains multiple Ig V-like domains in tandem and between Ig C-like domains where the cell surface receptor contains multiple tandem Ig C-like regions are also contemplated as hinges useful in single chain polypeptides of polypeptide heterodimers. In certain embodiments, hinge sequences comprised of cell surface receptor interdomain regions may further contain a naturally occurring or added motif, such as an IgG core hinge sequence that confers one or more disulfide bonds to stabilize the polypeptide heterodimer formation. Examples of hinges include interdomain regions between the Ig V-like and Ig C-like regions of CD2, CD4, CD22, CD33, CD48, CD58, CD66, CD80, CD86, CD150, CD166, and CD244.

In certain embodiments, hinge sequences have about 5 to 150 amino acids, 5 to 10 amino acids, 10 to 20 amino acids, 20 to 30 amino acids, 30 to 40 amino acids, 40 to 50 amino acids, 50 to 60 amino acids, 5 to 60 amino acids, 5 to 40 amino acids, for instance, about 8 to 20 amino acids and about 10 to 15 amino acids. The hinges may be primarily flexible, but may also provide more rigid characteristics or may contain primarily α-helical structure with minimal β-sheet structure. The lengths or the sequences of the hinges may affect the binding affinities of the binding domains to which the hinges are directly or indirectly (via another region or domain, such as an immunoglobulin heterodimerization domain) connected as well as one or more activities of the Fc region portions to which the hinges are directly or indirectly connected.

In one embodiment, hinge sequences are stable in plasma and serum and are resistant to proteolytic cleavage. The first lysine in the IgG1 upper hinge region may be mutated to minimize proteolytic cleavage, for instance, the lysine may be substituted with methionine, threonine, alanine or glycine, or may be deleted (see, e.g., SEQ ID NOS:826-881, which may include junction amino acids at the amino terminus such as RT).

In some embodiments, hinge sequences may contain a naturally occurring or added motif such as an immunoglobulin hinge core structure CPPCP (SEQ ID NO:228) that confers the capacity to form a disulfide bond or multiple disulfide bonds to stabilize the carboxy-terminus of a molecule. In other embodiments, hinge sequences may contain one or more glycosylation sites.

Exemplary hinges, including altered immunoglobulin hinges, are set forth in SEQ ID NOS:618-749 and 796-881.

In certain embodiments where a single chain polypeptide of a polypeptide heterodimer comprises a binding domain at or near its carboxyl terminus, a hinge may be present to link the binding domain with another portion of the single chain polypeptide (e.g., an Fc region portion or an immunoglobulin heterodimerization domain). Such a hinge may be a non-immunoglobulin hinge (i.e., a hinge not based on or derived from a wild type immunoglobulin hinge), a stalk region of a type II C-lectin or CD molecule, an interdomain region that connect IgV-like or IgC-like domains of a cell surface receptor, or a derivative or functional variant thereof. Exemplary carboxyl terminal hinges, sometimes referred to as “back-end” hinges, includes those set forth in SEQ ID NOS:734-737, 742-747, 799-823, and 825.

In certain embodiments, a hinge of one single chain polypeptide of a polypeptide heterodimer is identical to a corresponding hinge of the other single chain polypeptide of the heterodimer. In certain other embodiments, a hinge of one chain is different from that of the other chain (in their length or sequence).

### Other Components or Modifications

In certain embodiments, a single chain polypeptide that forms a heterodimer with another single chain polypeptide may contain one or more additional domains or regions. Such additional regions may be a leader sequence (also referred to as “signal peptide”) at the amino-terminus for secretion of an expressed single chain polypeptide. Exemplary leader peptides of this disclosure include natural leader sequences or others, such as those as set forth in SEQ ID NOS:110 and 111.

Additional regions may also be sequences at the carboxy-terminus for identifying or purifying single chain polypeptides (e.g., epitope tags for detection or purification, such as a histidine tag, biotin, a FLAG® epitope, or any combination thereof).

Further optional regions may be additional amino acid residues (referred to as “junction amino acids” or “junction amino acid residues”) having a length of one to about 5 amino acids, which result from use of specific expression systems or construct design for the single chain polypeptides of the present disclosure. Such additional amino acid residues (for instance, one, two, three, four or five additional amino acids) may be present at the amino or carboxyl terminus or between various regions or domains of a single chain polypeptide, such as between a binding domain and an immunoglobulin heterodimerization domain, between an immunoglobulin heterodimerization domain and a hinge, between a hinge and an Fc region portion, between domains of an Fc region portion (e.g., between CH2 and CH3 domains or between two CH3 domains), between a binding domain and a hinge, between an Fc region portion and an immunoglobulin heterodimerization domain, or between a variable domain and a linker. Exemplary junction amino acids amino-terminal to a hinge include RDQ (SEQ ID NO:598), RT, SS, SASS (SEQ ID NO:599) and SSS (SEQ ID NO:600). Exemplary junction amino acids carboxy-terminal to a hinge include amino acids SG. Additional exemplary junction amino acids include SR.

In certain embodiments, junction amino acids are present between an Fc region portion that comprises CH2 and CH3 domains and an immunoglobulin heterodimerization domain (CH1 or CL). These junction amino acids are also referred to as a “linker between CH3 and CH1 or CL” if they are present between the C-terminus of CH3 and the N-terminus of CH1 or CL. Such a linker may be 2-10 amino acids in length. In certain embodiments, the Fc region portion comprises human IgG1 CH2 and CH3 domains in which the C-terminal lysine residue of human IgG1 CH3 is deleted. Exemplary linkers between CH3 and CH1 include those set forth in SEQ ID NO:788-790. Exemplary linkers between CH3 and Ck include those set forth in SEQ ID NOS:791-793 (in which the carboxyl terminal arginine in the linkers may alternatively be regarded as the first arginine of Ck). In certain embodiments, the presence of such linkers or linker pairs (e.g., SEQ ID NO:788 as a CH3-CH1 linker in one single chain polypeptide of a heterodimer and SEQ ID NO:791 as a CH3-Ck linker in the other single chain polypeptide of the heterodimer; SEQ ID NO:789 as a CH3-CH1 linker and SEQ ID NO:792 as a CH3-Ck linker; and SEQ ID NO:790 as a CH3-CH1 linker and SEQ ID NO:793 as a CH3-Ck linker) improves the production of heterodimer compared the presence of a reference linker as set forth in SEQ ID NO:787 (in which the last lysine of CH3 is included as part of the linker) in both single chain polypeptides of a heterodimer.

In certain embodiments, an immunoglobulin Fc region (e.g., CH2, CH3, and/or CH4 regions) of a polypeptide heterodimer of the present disclosure may have an altered glycosylation pattern relative to an immunoglobulin reference sequence. For example, any of a variety of genetic techniques may be employed to alter one or more particular amino acid residues that form a glycosylation site (see Co et al. (1993) Mol. Immunol. 30:1361; Jacquemon et al. (2006) J. Thromb. Haemost. 4:1047; Schuster et al. (2005) Cancer Res. 65:7934; Warnock et al. (2005) Biotechnol. Bioeng. 92:831), such as N297 of the CH2 domain (EU numbering). Alternatively, the host cells producing polypeptide heterodimers of this disclosure may be engineered to produce an altered glycosylation pattern. One method known in the art, for example, provides altered glycosylation in the form of bisected, non-fucosylated variants that increase ADCC. The variants result from expression in a host cell containing an oligosaccharide-modifying enzyme. Alternatively, the Potelligent technology of BioWa/Kyowa Hakko is contemplated to reduce the fucose content of glycosylated molecules according to this disclosure. In one known method, a CHO host cell for recombinant immunoglobulin production is provided that modifies the glycosylation pattern of the immunoglobulin Fc region, through production of GDP-fucose.

Alternatively, chemical techniques are used to alter the glycosylation pattern of polypeptide heterodimers of this disclosure. For example, a variety of glycosidase and/or mannosidase inhibitors provide one or more of desired effects of increasing ADCC activity, increasing Fc receptor binding, and altering glycosylation pattern. In certain embodiments, cells expressing polypeptide heterodimers of the instant disclosure are grown in a culture medium comprising a carbohydrate modifier at a concentration that increases the ADCC of immunoglycoprotein molecules produced by said host cell, wherein said carbohydrate modifier is at a concentration of less than 800 μM. In one embodiment, the cells expressing these polypeptide heterodimers are grown in a culture medium comprising castanospermine or kifunensine, for instance, castanospermine at a concentration of about 100-800 μM, such as 100 μM, 200 μM, 300 μM, 400 μM, 500 μM, 600 μM, 700 μM, or 800 μM. Methods for altering glycosylation with a carbohydrate modifier such as castanospermine are provided in U.S. Pat. No. 7,846,434 or PCT Publication No. WO 2008/052030.

### Structural Arrangements

As described herein, a polypeptide heterodimer of the present disclosure is formed at least substantially via the interaction between the immunoglobulin heterodimerization domains of two different single chain polypeptides. For example, a first single chain polypeptide can comprise a binding domain that specifically binds a target, a hinge, a first immunoglobulin heterodimerization domain, and an Fc region portion, whereas a second single chain polypeptide can comprise a hinge, a second immunoglobulin heterodimerization domain that is different from the first immunoglobulin heterodimerization domain, and an Fc region portion but will lack a binding domain. To form a heterodimer comprising a single binding domain, the two single chain polypeptides are designed so that the first immunoglobulin heterodimerization domain of the first single chain polypeptide is properly aligned and interacts with the second immunoglobulin heterodimerization domain of the second single chain polypeptide. In addition to the interaction between the two immunoglobulin heterodimerization domains, in certain embodiments, an Fc region portion (e.g., a CH3 domain) in the first chain may interact with an identical portion of an Fc region in the second chain to facilitate heterodimerization. Moreover, in certain embodiments, the hinge in the first chain (e.g., an altered human IgG1 hinge with two cysteine residues as set forth in SEQ ID NO:229) may interact with the hinge in the second chain (e.g., the same altered human IgG1 hinge as set forth in SEQ ID NO:229) to form, for example, disulfide bonds, which may further facilitate or strengthen the interaction between the first and second single chain polypeptides to form a polypeptide heterodimer of the present disclosure.

A description of how various components can be arranged to make first and second polypeptides that form polypeptide heterodimers of the present disclosure is provided herein. In all the following exemplary arrangements, hinges of both the first and second single chain polypeptides are located amino terminal to the Fc region portion in those single chain polypeptides (although as described above, one or a few junction amino acids may be present between a hinge and an Fc region portion). However, it is contemplated that in certain embodiments, hinges of both the first and second single chain polypeptides may be located between the immunoglobulin heterodimerization domain and the Fc region portion. It is also contemplated that in certain embodiments the hinge of the first single chain polypeptide will be located between the binding domain and the first immunoglobulin heterodimerization domain, and the hinge of the second single chain polypeptide will be connected to the amino terminus of the second immunoglobulin heterodimerization domain in the same orientation as the hinge of the first single chain polypeptide. For example, if the hinge of the first single chain polypeptide is located amino terminal to the first immunoglobulin heterodimerization domain, then the hinge of the second single chain polypeptide will also be located amino terminal to the second immunoglobulin heterodimerization domain. Similarly, if the hinge of the first single chain polypeptide is located carboxyl terminal to the first immunoglobulin heterodimerization domain, then the hinge of the second single chain polypeptide will also be located carboxyl terminal to the second immunoglobulin heterodimerization domain.

In the following exemplary arrangements, hinges of the first and second single chain polypeptides are immunoglobulin hinges. However, it is contemplated that in certain embodiments, hinges of the first and second single chain polypeptides may be hinges that are not derived from immunoglobulin hinges, such as lectin interdomain regions or cluster of differentiation molecule stalk regions as described herein.

In one embodiment, a polypeptide heterodimer comprises the following two single chain polypeptides: from amino terminus to carboxyl terminus, a first or long single chain polypeptide comprising a binding domain, a CH1 region, an immunoglobulin hinge, and an Fc region portion; and a second or short single chain polypeptide comprising a CL region (e.g., Cκ, Cλ), an immunoglobulin hinge, and an Fc region portion.

In another embodiment, a polypeptide heterodimer comprises the following two single chain polypeptides: from amino terminus to carboxyl terminus, a first or long single chain polypeptide comprising a binding domain, an immunoglobulin hinge, an Fc region portion, and a CH1 region; and a second or short single chain polypeptide comprising an immunoglobulin hinge, an Fc region portion, and a CL region.

In another embodiment, a polypeptide heterodimer comprises the following two single chain polypeptides: from amino terminus to carboxyl terminus, a first or long single chain polypeptide comprising a binding domain, a CH1 region, an immunoglobulin hinge, an Fc region portion, and a second CH1 region: and a second single chain polypeptide comprising a CL region, an immunoglobulin hinge, an Fc region portion, and a second CL region.

In another embodiment, a polypeptide heterodimer comprises the following two single chain polypeptides: from amino terminus to carboxyl terminus, a first single chain polypeptide comprising a binding domain, a CH1 region, a second CH1 region, an immunoglobulin hinge, and an Fc region portion; and a second single chain polypeptide comprising a CL region, a second CL region, an immunoglobulin hinge and an Fc region portion.

In another embodiment, a polypeptide heterodimer comprises the following two single chain polypeptides: from amino terminus to carboxyl terminus, a first single chain polypeptide comprising a binding domain, an immunoglobulin hinge, an Fc region portion, a CH1 region, and a second CH1 region; and a second single chain polypeptide comprising an immunoglobulin hinge, an Fc region portion, a CL region and a second CL region.

In another embodiment, a polypeptide heterodimer comprises the following two single chain polypeptides: from amino terminus to carboxyl terminus, a first single chain polypeptide comprising a CH1 region, an immunoglobulin hinge, an Fc region portion and a binding domain; and a second single chain polypeptide comprising a CL region, an immunoglobulin hinge, and an Fc region portion.

In another embodiment, a polypeptide heterodimer is formed from the following two single chain polypeptides: from amino terminus to carboxyl terminus, a first single chain polypeptide comprising an immunoglobulin hinge, an Fc region portion, a CH1 region, and a binding domain; and a second single chain polypeptide comprising an immunoglobulin hinge, an Fc region portion, and a CL region.

In another embodiment, a polypeptide heterodimer comprises the following two single chain polypeptides: from amino terminus to carboxyl terminus, a first single chain polypeptide comprising a CH1 region, an immunoglobulin hinge, an

Fc region portion, a second CH1 region, and a binding domain; and a second single chain polypeptide comprising a CL region, an immunoglobulin hinge, an Fc region portion, and a second CL region.

In another embodiment, a polypeptide heterodimer comprises the following two single chain polypeptides: from amino terminus to carboxyl terminus, a first single chain polypeptide comprising a CH1 region, a second CH1 region, an immunoglobulin hinge, an Fc region portion, and a binding domain; and a second single chain polypeptide comprising a CL region, a second CL region, an immunoglobulin hinge and an Fc region portion.

In another embodiment, a polypeptide heterodimer comprises the following two single chain polypeptides: from amino terminus to carboxyl terminus, a first single chain polypeptide comprising an immunoglobulin hinge, an Fc region portion, a CH1 region, a second CH1 region, and a binding domain; and a second single chain polypeptide comprising an immunoglobulin hinge, an Fc region portion, a CL region and a second CL region.

In another embodiment, a polypeptide heterodimer comprises the following two single chain polypeptides: from amino terminus to carboxyl terminus, a first single chain polypeptide comprising a binding domain, a CL region, an immunoglobulin hinge, and an Fc region portion; and a second single chain polypeptide comprising a CH1 region, an immunoglobulin hinge, and an Fc region portion.

In another embodiment, a polypeptide heterodimer comprises the following two single chain polypeptides: from amino terminus to carboxyl terminus, a first single chain polypeptide comprising a binding domain, an immunoglobulin hinge, an Fc region portion, and a CL region; and a second single chain polypeptide comprising an immunoglobulin hinge, an Fc region portion, and a CH1 region.

In another embodiment, a polypeptide heterodimer comprises the following two single chain polypeptides: from amino terminus to carboxyl terminus, a first single chain polypeptide comprising a binding domain, a CL region, an immunoglobulin hinge, an Fc region portion, and a second CL region; and a second single chain polypeptide comprising a CH1 region, an immunoglobulin hinge, an Fc region portion, and a second CH1 region.

In another embodiment, a polypeptide heterodimer comprises the following two single chain polypeptides: from amino terminus to carboxyl terminus, a first single chain polypeptide comprising a binding domain, a CL region, a second CL region, an immunoglobulin hinge, and an Fc region portion; and a second single chain polypeptide comprising a CH1 region, a second CH1 region, an immunoglobulin hinge, and an Fc region portion.

In another embodiment, a polypeptide heterodimer comprises the following two single chain polypeptides: from amino terminus to carboxyl terminus, a first single chain polypeptide comprising a binding domain, an immunoglobulin hinge, an Fc region portion, a CL region, and a second CL region; and a second single chain polypeptide comprising an immunoglobulin hinge, an Fc region portion, a CH1 region, and a second CH1 region.

In another embodiment, a polypeptide heterodimer comprises the following two single chain polypeptides: from amino terminus to carboxyl terminus, a first single chain polypeptide comprising a CL region, an immunoglobulin hinge, an Fc region portion, and a binding domain; and a second single chain polypeptide comprising a CH1 region, an immunoglobulin hinge, and an Fc region portion.

In another embodiment, a polypeptide heterodimer comprises the following two single chain polypeptides: from amino terminus to carboxyl terminus, a first single chain polypeptide comprising an immunoglobulin hinge, an Fc region portion, a CL region, and a binding domain; and a second single chain polypeptide comprising an immunoglobulin hinge, an Fc region portion, and a CH1 region.

In another embodiment, a polypeptide heterodimer comprises the following two single chain polypeptides: from amino terminus to carboxyl terminus, a first single chain polypeptide comprising a CL region, an immunoglobulin hinge, an Fc region portion, a second CL region, and a binding domain; and a second single chain polypeptide comprising a CH1 region, an immunoglobulin hinge, an Fc region portion, and a second CH1 region.

In another embodiment, a polypeptide heterodimer comprises the following two single chain polypeptides: from amino terminus to carboxyl terminus, a first single chain polypeptide comprising a CL region, a second CL region, an immunoglobulin hinge, an Fc region portion, and a binding domain; and a second single chain polypeptide comprising a CH1 region, a second CH1 region, an immunoglobulin hinge, and an Fc region portion.

In another embodiment, a polypeptide heterodimer comprises the following two single chain polypeptides: from amino terminus to carboxyl terminus, a first single chain polypeptide comprising an immunoglobulin hinge, an Fc region portion, a CL region, a second CL region, and a binding domain; and a second single chain polypeptide comprising an immunoglobulin hinge, an Fc region portion, a CH1 region, and a second CH1 region.

In another embodiment, a polypeptide heterodimer comprises the following two single chain polypeptides: from amino terminus to carboxyl terminus, a first single chain polypeptide comprising a binding domain, a CH1 region, an immunoglobulin hinge, an Fc region portion, and a CL region; and a second single chain polypeptide comprising a CL region, an immunoglobulin hinge, an Fc region portion, and a CH1 region.

In another embodiment, a polypeptide heterodimer comprises the following two single chain polypeptides: from amino terminus to carboxyl terminus, a first single chain polypeptide comprising a binding domain, a CL region, an immunoglobulin hinge, an Fc region portion, and a CH1 region; and a second single chain polypeptide comprising a CH1 region, an immunoglobulin hinge, an Fc region portion, and a CL region.

In another embodiment, a polypeptide heterodimer comprises the following two single chain polypeptides: from amino terminus to carboxyl terminus, a first single chain polypeptide comprising a binding domain, a CH1 region, a CL region, an immunoglobulin hinge, and an Fc region portion; and a second single chain polypeptide comprising a CL region, a CH1 region, an immunoglobulin hinge and an Fc region portion.

In another embodiment, a polypeptide heterodimer comprises the following two single chain polypeptides: from amino terminus to carboxyl terminus, a first single chain polypeptide comprising a binding domain, a CL region, a CH1 region, an immunoglobulin hinge, and an Fc region portion; and a second single chain polypeptide comprising a CH1 region, a CL region, an immunoglobulin hinge and an Fc region portion.

In another embodiment, a polypeptide heterodimer comprises the following two single chain polypeptides: from amino terminus to carboxyl terminus, a first single chain polypeptide comprising a binding domain, an immunoglobulin hinge, an Fc region portion, a CH1 region and a CL region; and a second single chain polypeptide comprising an immunoglobulin hinge, an Fc region portion, a CL region and a CH1 region.

In another embodiment, a polypeptide heterodimer comprises the following two single chain polypeptides: from amino terminus to carboxyl terminus, a first single chain polypeptide comprising a binding domain, an immunoglobulin hinge, an Fc region portion, a CL region and a CH1 region; and a second single chain polypeptide comprising an immunoglobulin hinge, an Fc region portion, a CH1 region and a CL region.

In another embodiment, a polypeptide heterodimer comprises the following two single chain polypeptides: from amino terminus to carboxyl terminus, a first single chain polypeptide comprising a CH1 region, an immunoglobulin hinge, an Fc region portion, a CL region, and a binding domain; and a second single chain polypeptide comprising a CL region, an immunoglobulin hinge, an Fc region portion, and a CH1 region.

In another embodiment, a polypeptide heterodimer comprises the following two single chain polypeptides: from amino terminus to carboxyl terminus, a first single chain polypeptide comprising a CL region, an immunoglobulin hinge, an Fc region portion, a CH1 region, and a binding domain; and a second single chain polypeptide comprising a CH1 region, an immunoglobulin hinge, an Fc region portion, and a CL region.

In another embodiment, a polypeptide heterodimer comprises the following two single chain polypeptides: from amino terminus to carboxyl terminus, a first single chain polypeptide comprising a CH1 region, a CL region, an immunoglobulin hinge, an Fc region portion, and a binding domain; and a second single chain polypeptide comprising a CL region, a CH1 region, an immunoglobulin hinge and an Fc region portion.

In another embodiment, a polypeptide heterodimer comprises the following two single chain polypeptides: from amino terminus to carboxyl terminus, a first single chain polypeptide comprising a CL region, a CH1 region, an immunoglobulin hinge, an Fc region portion, and a binding domain; and a second single chain polypeptide comprising a CH1 region, a CL region, an immunoglobulin hinge and an Fc region portion.

In another embodiment, a polypeptide heterodimer comprises the following two single chain polypeptides: from amino terminus to carboxyl terminus, a first single chain polypeptide comprising an immunoglobulin hinge, an Fc region portion, a CH1 region, a CL region, and a binding domain; and a second single chain polypeptide comprising an immunoglobulin hinge, an Fc region portion, a CL region and a CH1 region.

In another embodiment, a polypeptide heterodimer comprises the following two single chain polypeptides: from amino terminus to carboxyl terminus, a first single chain polypeptide comprising an immunoglobulin hinge, an Fc region portion, a CL region, a CH1 region, and a binding domain; and a second single chain polypeptide comprising an immunoglobulin hinge, an Fc region portion, a CH1 region and a CL region.

In any of the embodiments described herein, the binding domain may be on either the first single chain polypeptide or on the second single chain polypeptide. In other words, the first single chain polypeptide will be the long polypeptide (having a binding domain) when the second single chain polypeptide is the short (lacking a binding domain) polypeptide. Conversely, the first single chain polypeptide will be the short polypeptide (lacking a binding domain) when the second single chain polypeptide is the long (having a binding domain) polypeptide.

Exemplary heterodimers may be formed from single chain polypeptide pairs described herein. If sequence identification numbers noted herein contain signal peptide sequences (e.g., the first 20 amino acids), such signal peptide sequences are not part of the mature single chain polypeptides that form the exemplary polypeptide heterodimers and thus should be considered excluded.

As described herein, a first or long single chain polypeptide of a polypeptide heterodimer comprises a binding domain, a hinge, an immunoglobulin heterodimerization domain (e.g., CH1, CL), and an Fc region portion. Exemplary first single chain polypeptides (with or without a signal peptide sequence) are set forth in SEQ ID NOS:22, 26, 30, 36, 38, 42, 46, 56, 60, 62, 70, 135, 139, 263, 267, 769, 780, and 781. In certain embodiments, the first single chain polypeptide may further comprise an additional immunoglobulin heterodimerization domain.

Also as described herein, a second or short single chain polypeptide of a polypeptide heterodimer comprises a hinge, an immunoglobulin heterodimerization domain, and an Fc region portion. Exemplary second single chain polypeptides (either with or without a signal peptide sequence) as set forth in SEQ ID NOS:24, 28, 32, 34, 40, 44, 48, 50, 52, 54, 58, 64, 66, 68, 72-105, 127, 129, 131, 133, 137, 193, 765-768, 778 and 779. In certain embodiments, the second single chain polypeptide may further comprise an additional immunoglobulin heterodimerization domain.

Exemplary heterodimers may be formed from the following single chain polypeptide pairs: SEQ ID NOS: 22 and 24, 26 and 28, 30 and 32, 36 and 34, 38 and 40, 42 and 44, 46 and 48, 56 and 54, 60 and 58, 26 and 52, 70 and 68, 46 and 70, 22 and 50, 62 and 64, 38 and 66, 46 and 64, 62 and 48, 22 and 127, 22 and 129, 22 and 131, 22 and 133, 135 and 24, 135 and 133, 135 and 131, 26 and 137, 139 and 48, 263 and 48, 267 and 48, 769 and 765, 769 and 766, 769 and 767, 769 and 768, 778 and 781, and 779 and 780. Additional exemplary heterodimers may be formed from a first chain as set forth in SEQ ID NO:22 (but without its signal peptide sequence) and a second chain selected from SEQ ID NOS:72-105 and 193.

Exemplary heterodimers may be formed from the following single chain pairs: SEQ ID NOS:26 and 137, 139 and 48, 46 and 48, 46 and 64, 62 and 48, and 62 and 64. Additional heterodimers may be formed from a first chain as set forth in SEQ ID NO:22 (but without its signal peptide sequence) and a second chain selected from SEQ ID NOS:91, 92, 193, 98, 99, 101, 103, 127, 129, 131, and 133. Heterodimers may also be formed from a first chain as set froth in SEQ ID NO:135 and a second chain selected from SEQ ID NOS:24, 133 and 131.

### Nucleic Acids Encoding Single Chain Polypeptides, Vectors, Host Cells, and Methods for Making Heterodimers

In a related aspect, the present disclosure also provides isolated nucleic acid (used interchangeably with “polynucleotide”) molecules that encode single chain polypeptides provided herein. Exemplary nucleic acid molecules (either with or without a nucleotide sequence encoding a signal peptide sequence) are set forth in SEQ ID NOS: 21, 23, 25, 27, 29, 31, 33, 35, 37, 39, 41, 43, 45, 47, 49, 51, 53, 55, 57, 59, 61, 63, 65, 67, 69, 126, 128, 130, 132, 134, 136, 138, 760-764, and 774-777.

The present disclosure also provides vectors that comprise nucleic acid sequence encoding single chain polypeptides provided herein. As used herein, “vector” refers to a nucleic acid molecule capable of transporting another nucleic acid to which it has been linked. Exemplary vectors include plasmids, yeast artificial chromosomes, and viral genomes. Certain vectors can autonomously replicate in a host cell, while other vectors can be integrated into the genome of a host cell and thereby are replicated with the host genome.

In certain embodiments, the vectors may be recombinant expression vectors. “Recombinant expression vectors” or “expression vectors” refer to vectors that contain nucleic acid sequences that are operatively linked to an expression control sequence (e.g., a promoter) and are thus capable of directing the expression of those sequences.

Promoter sequences useful in expression vectors provided herein can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. In certain embodiments, the promoters are inducible promoters.

In certain embodiments, a vector is an expression vector that comprises a nucleic acid sequence encoding a first single chain polypeptide of a polypeptide heterodimer provided herein. In certain other embodiments, a vector is an expression vector that comprises a nucleic acid sequence encoding a second single chain polypeptide of a polypeptide heterodimer provided herein.

In certain embodiments, a vector is an expression vector that comprises nucleic acid sequences encoding both first and second single chain polypeptides of a polypeptide heterodimer. The promoter for the nucleic acid sequence encoding the first single chain polypeptide may be the same as the promoter for the nucleic acid encoding the second single chain polypeptide. Alternatively, the promoter for the nucleic acid sequence encoding the first single chain polypeptide may be different from the promoter for the nucleic acid encoding the second single chain polypeptide so that the expression level of the first and second single chain polypeptides may be differentially modulated to maximum heterodimerization of the first and second single chain polypeptides. In certain embodiments, one or both the promoters for the nucleic acid encoding the first and second single chain polypeptides are inducible promoters.

The present disclosure also provides a host cell transformed or transfected with, or otherwise containing, any of the nucleic acids or vectors provided herein. Exemplary host cells include VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, Escherichia coli, Bacillus subtilis, Salmonella typhimurium, and a member of the Streptomycete family.

In certain embodiments, a host cell comprises a first expression vector containing a nucleic acid encoding a first single chain polypeptide and a second expression vector containing a nucleic acid encoding a second single chain polypeptide.

In certain other embodiments, a host cell comprises an expression vector containing a nucleic acid encoding both first and second single chain polypeptides.

The disclosure also includes a method of producing polypeptide heterodimers described herein. In certain embodiments, the method comprises culturing a host cell that comprises nucleic acids encoding both the first and second single chain polypeptides under conditions suitable to express the polypeptides, and optionally isolating or purifying the heterodimers formed from the first and second single chain polypeptides from the culture. The nucleic acid encoding the first single chain polypeptide and the nucleic acid encoding the second single chain polypeptide may be present in a single expression vector in the host cell or in two different expression vectors in the host cells. In the latter case, the ratio between the two expression vectors may be controlled to maximize heterodimerization of the first and second single chain polypeptides.

The present disclosure provides purified polypeptide heterodimers as described herein. The term “purified,” as used herein, refers to a composition, isolatable from other components, wherein the polypeptide heterodimer is enriched to any degree relative to its naturally obtainable state. In certain embodiments, the present disclosure provides substantially purified polypeptide heterodimers as described herein. “Substantially purified” refers to a polypeptide heterodimer composition in which the polypeptide heterodimer forms the major component of the composition, such as constituting at least about 50%, such as at least about 60%, about 70%, about 80%, about 90%, about 95%, about 99%, of the polypeptides, by weight, in the composition.

Protein purification techniques are well known to those of skill in the art. These techniques involve, at one level, the crude fractionation of the polypeptide and non-polypeptide fractions. Further purification using chromatographic and electrophoretic techniques to achieve partial or complete purification (or purification to homogeneity) is frequently desired. Analytical methods particularly suited to the preparation of a pure fusion protein are ion-exchange chromatography, size exclusion chromatography; polyacrylamide gel electrophoresis; and isoelectric focusing. Particularly efficient methods of purifying peptides are fast protein liquid chromatography and HPLC.

Various methods for quantifying the degree of purification are known to those of skill in the art in light of the present disclosure. These include, for example, assessing the amount of polypeptide heterodimers in a fraction by SDS/PAGE analysis and HPLC as illustrated in the examples provided herein.

The method for making polypeptide heterodimers provided herein is advantageous over a method for first expressing and purifying separately individual single chain polypeptides and then incubating purified individual single chain polypeptides together to form polypeptide heterodimers. For example, certain single chain polypeptides (e.g., certain polypeptides containing only CH1 regions as their immunoglobulin heterodimerization domains) are unstable when expressed alone. In addition, separate expression and purification of individual single chain polypeptides followed by combining the purified individual single chain polypeptides involve more steps than coexpressing both single chain polypeptides followed by purifying resulting polypeptide heterodimers and generally less efficient.

### Compositions and Methods for Using Heterodimers

In addition to polypeptide heterodimers, the present disclosure also provides pharmaceutical compositions and unit dose forms that comprise the polypeptide heterodimers as well as methods for using the polypeptide heterodimers, the pharmaceutical compositions and unit dose forms.

Compositions of polypeptide heterodimers of this disclosure generally comprise a polypeptide heterodimer provided herein in combination with a pharmaceutically acceptable excipient, including pharmaceutically acceptable carriers and diluents. Pharmaceutical acceptable excipients will be nontoxic to recipients at the dosages and concentrations employed. They are well known in the pharmaceutical art and described, for example, in Rowe et al., Handbook of Pharmaecutical Excipients: A Comprehensive Guid to Uses, Properties, and Safety, 5^{th} Ed., 2006.

Pharmaceutically acceptable carriers for therapeutic use are also well known in the pharmaceutical art, and are described, for example, in Remington's Pharmaceutical Sciences, Mack Publishing Co. (A. R. Gennaro (Ed.) 1985). Exemplary pharmaceutically acceptable carriers include sterile saline and phosphate buffered saline at physiological pH. Preservatives, stabilizers, dyes and the like may be provided in the pharmaceutical composition. In addition, antioxidants and suspending agents may also be used.

Pharmaceutical compositions may also contain diluents such as buffers, antioxidants such as ascorbic acid, low molecular weight (less than about 10 residues) polypeptides, proteins, amino acids, carbohydrates (e.g., glucose, sucrose, dextrins), chelating agents (e.g., EDTA), glutathione and other stabilizers and excipients. Neutral buffered saline or saline mixed with nonspecific serum albumin are exemplary diluents. For instance, the product may be formulated as a lyophilizate using appropriate excipient solutions (e.g., sucrose) as diluents.

The present disclosure also provides a method for treating a disease or disorder associated with, for example, excessive receptor-mediated signal transduction, comprising administering to a patient in need thereof an effective amount of a polypeptide heterodimer comprising a binding domain that specifically binds a receptor.

Exemplary diseases or disorders associated with excess receptor-mediated signal transduction include cancer (e.g., solid malignancy and hematologic malignancy), autoimmune or inflammatory diseases or conditions, sepsis resulting from bacterial infection, and viral infection.

The present disclosure also provides a method for reducing T cell activation comprising administering to a patient in need thereof an effective amount of a polypeptide heterodimer provided herein that specifically binds CD28. A treatment “reduces T cell activation” if it causes statistically reduction of T cell activation. Assays for measuring T cell activation are known in the art, such as those used in the examples provided herein.

In one aspect, the present disclosure provides a method for inhibiting growth of a solid malignancy, inhibiting metastasis or metastatic growth of a solid malignancy, or treating or ameliorating a hematologic malignancy, comprising administering to a patient in need thereof an effective amount of a polypeptide heterodimer provided herein or a composition thereof

A wide variety of cancers, including solid malignancy and hematologic malignancy are amenable to the compositions and methods disclosed herein. Types of cancer that may be treated include, but are not limited to: adenocarcinoma of the breast, prostate, pancreas, colon and rectum; all forms of bronchogenic carcinoma of the lung (including squamous cell carcinoma, adenocarcinoma, small cell lung cancer and non-small cell lung cancer); myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin; and glioblastoma multiforme. The types of cancers that may be treated also include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia. The invention further provides compositions and methods useful in the treatment of other conditions in which cells have become immortalized or hyperproliferative due to abnormally high expression of antigen.

Additional exemplary cancers that are also amenable to the compositions and methods disclosed herein are B-cell cancers, including B-cell lymphomas [such as various forms of Hodgkin's disease, non-Hodgkins lymphoma (NHL) or central nervous system lymphomas], leukemias [such as acute lymphoblastic leukemia (ALL), chronic lymphocytic leukemia (CLL), Hairy cell leukemia and chronic myoblastic leukemia] and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt lymphoma/leukemia, B-cell proliferations of uncertain malignant potential, lymphomatoid granulomatosis, and post-transplant lymphoproliferative disorder.

Polypeptide heterodimers useful for inhibiting growth of a solid malignancy or metastasis or metastatic growth of a solid malignancy include those that specifically bind to, for example, EGFR, ErbB3, ErbB4, c-Met, RON, EphA2, IGF1R, VEGFR1, VEGFR2, VEGFR3, CD44v6, CD151, CEACAM6, TGFBR2, GHRHR, GHR, IL6R, gp130, TNFR2, PD1, TWEAK-R, OSMRβ, Patched-1, Frizzled, or Robo1.

Polypeptide heterodimers useful for inhibiting growth of a solid malignancy or metastasis or metastatic growth of a hematologic malignancy include those that specifically bind to, for example, EGFR, ErbB3, c-Met, RON, EphA2, IGF1R, TGFBR2, IL6R, gp130, TNFR2, PD1, OSMRβ, LTβR, CD19, CD80, CD81, or CD86.

In another aspect, the present disclosure provides a method for treating an autoimmune or inflammatory disease, disorder or condition, comprising administering to a patient in need thereof an effective amount of a polypeptide heterodimer provided herein or a composition thereof.

Exemplary autoimmune or inflammatory diseases, disorders or conditions that may be treated by the fusion proteins and compositions and unit dose forms thereof include, and are not limited to, inflammatory bowel disease (e.g., Crohn's disease or ulcerative colitis), diabetes mellitus (e.g., type I diabetes), dermatomyositis, polymyositis, pernicious anaemia, primary biliary cirrhosis, acute disseminated encephalomyelitis (ADEM), Addison's disease, ankylosing spondylitis, antiphospholipid antibody syndrome (APS), autoimmune hepatitis, Goodpasture's syndrome, Graves' disease, Guillain-Barré syndrome (GBS), Hashimoto's disease, idiopathic thrombocytopenic purpura, systemic lupus erythematosus, lupus nephritis, neuropsychiatric lupus, multiple sclerosis (MS), myasthenia gravis, pemphigus vulgaris, asthma, psoriatic arthritis, rheumatoid arthritis, Sjogren's syndrome, temporal arteritis (also known as “giant cell arteritis”), autoimmune hemolytic anemia, Bullous pemphigoid, vasculitis, coeliac disease, chronic obstructive pulmonary disease, endometriosis, Hidradenitis suppurativa, interstitial cystitis, morphea, scleroderma, narcolepsy, neuromyotonia, vitiligo, and autoimmune inner ear disease.

Polypeptide heterodimers useful for treating an autoimmune or inflammatory disease, disorder or condition include those that specifically bind to, for example, TGFBR2, IL6R, gp130, TNFR1, TNFR2, PD1, HVEM, OX40, CD40, CD137, TWEAK-R, LTβR, LIFRβ, OSMRβ, CD3, TCRα, TCRβ, CD19, CD28, CD80, CD81, CD86, TLR7, or TLR9.

In another aspect, the present disclosure provides a method for reducing the risk of sepsis associated with a bacterial infection, comprising administering to a patient in need thereof an effective amount of a polypeptide heterodimer provided herein or a composition thereof. Exemplary polypeptide heterodimers useful for such treatments include those that specifically bind to TLR9.

In another aspect, the present disclosure provides a method for treating viral infection, comprising administering to a patient in need thereof an effective amount of a polypeptide heterodimer provided herein or a composition thereof. Exemplary polypeptide heterodimers useful for such treatments include those that specifically bind to HVEM, OX40, or LTβR.

The polypeptide heterodimers or compositions thereof of the present disclosure may be administered orally, topically, transdermally, parenterally, by inhalation spray, vaginally, rectally, or by intracranial injection, or any combination thereof. In one embodiment, the polypeptide heterodimers or compositions thereof are administered parenterally. The term “parenteral,” as used herein, includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and/or surgical implantation at a particular site is contemplated as well. For instance, the invention includes administering polypeptide heterodimers or compositions thereof by intravenous injection.

The pharmaceutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.01 mg/kg and 1000 mg/kg (e.g., between 0.1 mg/kg and 100 mg/kg, or between 1 mg/kg and 10 mg/kg) body weight (which can be administered as a single dose, daily, weekly, monthly, or at any appropriate interval) of active ingredient may be administered depending on the potency of a polypeptide heterodimer of this disclosure.

Also contemplated is the administration of polypeptide heterodimers or compositions thereof in combination with a second agent. A second agent may be one accepted in the art as a standard treatment for a particular disease state or disorder, such as in cancer, inflammation, autoimmunity, and infection. Exemplary second agents contemplated include polypeptide heterodimers that bind to targets different from those that primary polypeptide heterodimers bind, polyclonal antibodies, monoclonal antibodies, immunoglobulin-derived fusion proteins, chemotherapeutics, ionizing radiation, steroids, NSAIDs, anti-infective agents, or other active and ancillary agents, or any combination thereof

In certain embodiments, a polypeptide heterodimer and a second agent act synergistically. In other words, these two compounds interact such that the combined effect of the compounds is greater than the sum of the individual effects of each compound when administered alone (see, e.g., Berenbaum, Pharmacol. Rev. 41:93, 1989).

In certain other embodiments, a polypeptide heterodimer and a second agent act additively. In other words, these two compounds interact such that the combined effect of the compounds is the same as the sum of the individual effects of each compound when administered alone.

Second agents useful in combination with polypeptide heterodimers or compositions thereof provided herein for reducing T cell activation may be steroids, NSAIDs, mTOR inhibitors (e.g., rapamycin (sirolimus), temsirolimus, deforolimus, everolimus, zotarolimus, curcumin, farnesylthiosalicylic acid), calcineurin inhibitors (e.g., cyclosporine, tacrolimus), anti-metabolites (e.g., mycophenolic acid, mycophenolate mofetil), polyclonal antibodies (e.g., anti-thymocyte globulin), monoclonal antibodies (e.g., daclizumab, basiliximab), and CTLA4-Ig fusion proteins (e.g., abatacept or belatacept).

Additional second agents useful for reducing T cell activation may be a polyclonal or monoclonal antibody, an immunoglobulin-derived fusion protein (e.g., scFv, SMIP™, PIMS, SCORPION™, and Xceptor fusion proteins), or a polypeptide heterodimer according to the present disclosure that specifically bind a T-cell specific molecule, such as CD3, CD28, PD-1, HVEM, BTLA, CD80, CD86, GITR, or TGFBR1.

Second agents useful for inhibiting growth of a solid malignancy, inhibiting metastasis or metastatic growth of a solid malignancy, or treating or ameliorating a hematologic malignancy include chemotherapeutic agents, ionizing radiation, and other anti-cancer drugs. Examples of chemotherapeutic agents contemplated as further therapeutic agents include alkylating agents, such as nitrogen mustards (e.g., mechlorethamine, cyclophosphamide, ifosfamide, melphalan, and chlorambucil); bifunctional chemotherapeutics (e.g., bendamustine); nitrosoureas (e.g., carmustine (BCNU), lomustine (CCNU), and semustine (methyl-CCNU)); ethyleneimines and methyl-melamines (e.g., triethylenemelamine (TEM), triethylene thiophosphoramide (thiotepa), and hexamethylmelamine (HMM, altretamine)); alkyl sulfonates (e.g., buslfan); and triazines (e.g., dacabazine (DTIC)); antimetabolites, such as folic acid analogues (e.g., methotrexate, trimetrexate, and pemetrexed (multi-targeted antifolate)); pyrimidine analogues (such as 5-fluorouracil (5-FU), fluorodeoxyuridine, gemcitabine, cytosine arabinoside (AraC, cytarabine), 5-azacytidine, and 2,2′-difluorodeoxycytidine); and purine analogues (e.g, 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA)); Type I topoisomerase inhibitors such as camptothecin (CPT), topotecan, and irinotecan; natural products, such as epipodophylotoxins (e.g., etoposide and teniposide); and vinca alkaloids (e.g., vinblastine, vincristine, and vinorelbine); anti-tumor antibiotics such as actinomycin D, doxorubicin, and bleomycin; radiosensitizers such as 5-bromodeozyuridine, 5-iododeoxyuridine, and bromodeoxycytidine; platinum coordination complexes such as cisplatin, carboplatin, and oxaliplatin; substituted ureas, such as hydroxyurea; and methylhydrazine derivatives such as N-methylhydrazine (MIH) and procarbazine.

In certain embodiments, second agents useful for inhibiting growth of a solid malignancy, inhibiting metastasis or metastatic growth of a solid malignancy, or treating or ameliorating a hematologic malignancy include polypeptide heterodimers according to the present disclosure that bind to cancer cell targets other than the target that the first polypeptide heterodimer binds. In certain other embodiments, second agents useful for such treatments include polyclonal antibodies, monoclonal antibodies, and immunoglobulin-derived fusion proteins that bind to cancer cell targets. Exemplary cancer cell targets are provided above in the context of describing targets of polypeptide heterodimers useful for the above-noted treatment.

Further therapeutic agents contemplated by this disclosure for treatment of autoimmune diseases are referred to as immunosuppressive agents, which act to suppress or mask the immune system of the individual being treated. Immunosuppressive agents include, for example, non-steroidal anti-inflammatory drugs (NSAIDs), analgesics, glucocorticoids, disease-modifying antirheumatic drugs (DMARDs) for the treatment of arthritis, or biologic response modifiers. Compositions in the DMARD description are also useful in the treatment of many other autoimmune diseases aside from rheumatoid arthritis.

Exemplary NSAIDs are chosen from the group consisting of ibuprofen, naproxen, naproxen sodium, Cox-2 inhibitors such as Vioxx and Celebrex, and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g. etanercept (Enbrel), adalimumab (Humira) and infliximab (Remicade)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

Additional second agents useful for treating an autoimmune or inflammatory disease, disorder or condition may be a polyclonal or monoclonal antibody, an immunoglobulin-derived fusion protein (e.g., scFv, SMIP, PIMS, SCORPION™, and XCEPTOR™ fusion proteins), or a polypeptide heterodimer according to the present disclosure that specifically bind a target associated with such a disease, disorder or condition. Examples of such targets are provided above in the context of targets of polypeptide heterodimers of the present disclosure useful in the above-noted treatment bind.

In certain embodiments, second agents useful for treating sepsis associated with bacterial infection include chloroquine and small molecule TLR9 inhibitors (see, e.g., Yasuda et al. (2008) Am. J. Physiol. Renal Physiol. 294:F1050-F1058), recombinant human activated protein C, insulin, colloid or crystalloid, vasoactive agents, corticosteroids (see, e.g., Hotchkiss and Karl (2003) New England Journal of Medicine 348:138-150) and inhibitory CpG DNA sequences (see, e.g., Krieg et al. (1998) Proc. Natl. Acad. Sci. USA 95:12631-12636).

In certain embodiments, second agents useful for treating viral infection include other antiviral agents. Examples of such other antiviral agents include acyclovir, valacyclovir and famciclovir that may be used with an HVEM-specific heterodimer, oseltamivir, zanamivir, amantadine and rimantadine that may be used with an OX40-specific heterodimer, and anti-HIV agents that may be used with an LTβR-specific heterodimer. Exemplary anti-HIV agents include Abacavir (formerly Ziagen), Agenerase (amprenavir), Aptivus® (tipranavir), and Crixivan (indinavir), Delavirdine (formerly Rescriptor), efavirenz (formerly Sustiva), Emtriva [emtricitabine (FTC)], Epivir (lamivudine), Fortovase (saquinavir), Fuzeon (enfuvirtide), Hivid (ddc/zalcitabine), INTELENCE™ (Etravirine), Isentress (raltegravir), Invirase (saquinavir), Kaletra (lopinavir), lamivudine, Lexiva (Fosamprenavir), Nevirapine (formerly Viramune), Norvir (ritonavir), PREZISTA (darunavir), Retrovir [AZT (zidovudine)], Reyataz (atazanavir; BMS-232632), SELZENTRY™ (maraviroc), Stavudine (formerly Zerit), Tenofovir DF, Trizivir, Truvada. Videx (ddl/didanosine), Viracept (nelfinavir), Viread (tenofovir disoproxil fumarate), and zidovudine. In certain other embodiments, second agents useful for such a treatment include polyclonal antibodies, monoclonal antibodies, and immunoglobulin-derived fusion proteins or a polypeptide heterodimer according to the present disclosure that bind to targets associated with viral infection. Exemplary targets associated with viral infection include HVEM, OX40 and LTβR.

It is contemplated the binding molecule composition and the second active agent may be given simultaneously in the same formulation. Alternatively, the second agents may be administered in a separate formulation but concurrently (i.e., given within less than one hour of each other).

In certain embodiments, the second active agent may be administered prior to administration of a polypeptide heterodimer or a composition thereof. Prior administration refers to administration of the second active agent at least one hour prior to treatment with the polypeptide heterodimer or the composition thereof. It is further contemplated that the active agent may be administered subsequent to administration of the binding molecule composition. Subsequent administration is meant to describe administration at least one hour after the administration of the polypeptide heterodimer or the composition thereof.

This disclosure contemplates a dosage unit comprising a pharmaceutical composition of this disclosure. Such dosage units include, for example, a single-dose or a multi-dose vial or syringe, including a two-compartment vial or syringe, one comprising the pharmaceutical composition of this disclosure in lyophilized form and the other a diluent for reconstitution. A multi-dose dosage unit can also be, e.g., a bag or tube for connection to an intravenous infusion device.

This disclosure also contemplates a kit comprising a pharmaceutical composition of this disclosure in unit dose, or multi-dose, container, e.g., a vial, and a set of instructions for administering the composition to patients suffering a disorder such as a disorder described above.

### EXAMPLES

### Example 1

### Making of Single Chain Binding Polypeptides and Polypeptide Heterodimers thereof

### 1. Introduction

This example describes various single chain polypeptides and polypeptide heterodimers thereof that contain a single binding domain and have immunoglobulin heterodimerization domain pairs of Cκ-CH1 or Cλ-CH1, or a combination of these pairs. In the simplest form, polypeptide heterodimers (also referred to as Interceptors) are made by co-expressing two unequal chains, one chain having a Cκ or Cλ domain and the other chain having a CH1 region. For example, the first chain polypeptide, designated the long chain, has a binding domain in the form of scFv and a CH1 heterodimerization domain, whereas the other chain, designated the short chain, lacks a binding domain but has a Cκ heterodimerization domain. Polypeptide heterodimers (Interceptors) will generally bind monovalently to targets and are ideal for blocking receptor/ligand or receptor/receptor interactions and preventing cell activation through receptor cross-linking Other various advantages over, for example, a Fab, include a longer serum half-life and ease of purification due to the presence of the Fc domains.

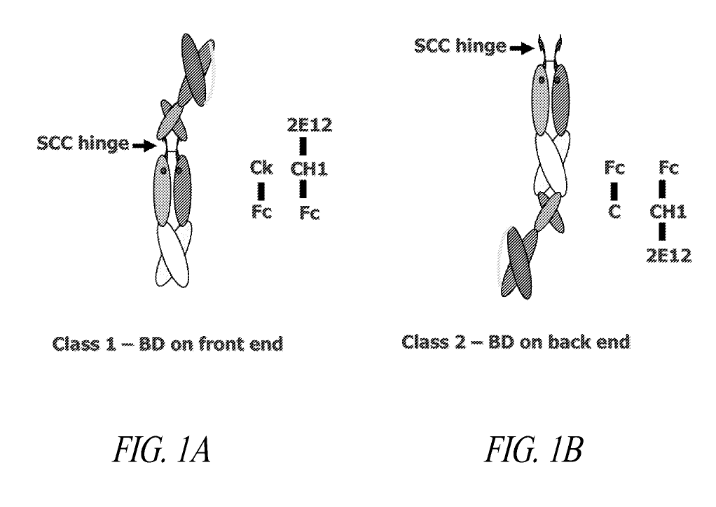

Class 1 Interceptors are those with a binding domain at the amino terminus, and Class 2 Interceptors are those with a binding domain at the carboxyl terminus (see, FIG. 1).

2. Methods

### 2.1 Construction of Single Chain Polypeptides Used to Make Interceptors

A small modular immunopharmaceutical protein (SMIP™) containing a 2E12 scFv is referred to as M0039. The DNA sequence of M0039 including a signal sequence (nucleotides 1-66) is set forth in SEQ ID NO:1. The XhoI and XbaI sites at positions 356-361 and 201-206 were subsequently mutated to CTCGGG and TCTGGA, respectively, without changing the amino acid sequence. The mutated M0039 was subsequently used as a template to build some of the molecules described herein. The amino acid sequence of mutated M0039 is set forth in SEQ ID NO:2. The first 22 amino acids of SEQ ID NO:2 are a signal peptide sequence, which is cleaved when the protein is exported from the host cell. The amino acid sequence of the signal peptide is also set forth in SEQ ID NO:110.

### 2.1.1 Construction of 2E12 CH1CH2CH3 (X0112)

First, the CH1 fragment (heterodimerization domain) was cloned by PCR using the oligonucleotides CH1xhonewF (SEQ ID NO:3) and CH1BsiwnewR (SEQ ID NO:4), with template X0038 (a construct containing the CH1 fragment). The PCR fragment was subsequently isolated and digested with XhoI and BsiWI restriction enzymes. Second, the CH2-CH3 Fc region portion was amplified by PCR using the oligonucleotides CH2BsiwnewF (SEQ ID NO:5) and CH3NotnewR (SEQ ID NO:6) with template M0077, an anti-CD79b SMIP construct containing wild type CH2CH3 domain sequences (Fc region portion). The Fc region fragment was then isolated and digested with XhoI and BsiWI restriction enzymes. The CH1 heterodimerization domain and Fc region fragments were then ligated into the pD28 vector using the XhoI and NotI restriction sites.

The 2E12 scFv binding domain was also cloned by PCR using the oligonucleotides 2E12AgeF (SEQ ID NO:7) and 2E12XhoR (SEQ ID NO:8) using the mutated M0039 as the template. The scFv fragment was isolated, digested with restriction enzymes AgeI and XhoI and ligated into the new pD28 vector as described above utilizing the AgeI and XhoI sites.

The nucleotide sequence encoding, and the amino acid sequence of, the resulting single chain fusion protein, X0112, are set forth in SEQ ID NOS:21 and 22, respectively. The first 60 nucleotides in SEQ ID NO:21 encode the first 20 amino acids, a signal peptide, in SEQ ID NO:22. The amino acid sequence of the signal peptide is also set forth in SEQ ID NO:111.

### 2.1.2 Construction of CKCH2CH3 (X0113)

First, the Cκ fragment was cloned by PCR using the oligonucleotides CkAgeIF (SEQ ID NO:9) and CkBsiWR (SEQ ID NO:10) with template X0033 containing the Ck domain. The PCR fragment was subsequently isolated and digested with AgeI and BsiWI restriction enzymes. Second, the CH2CH3 domain was cloned by PCR using the oligonucleotides CH2 BsiwnewF (SEQ ID NO:11) and CH3NotnewR (SEQ ID NO:12) with template M0077 containing the wild type CH2CH3. The fragment was then isolated and digested with BsiWI and NotI. The Cκ heterodimerization domain and Fc region fragments were then ligated into the pD28 vector using the AgeI and NotI sites.

The nucleotide sequence encoding, and the amino acid sequence of, the resulting single chain fusion protein, X0113, are set forth in SEQ ID NOS:23 and 24, respectively. The first 60 nucleotides in SEQ ID NO:23 encode a 20 amino acid signal peptide in SEQ ID NO:24. The Interceptor, X0124, was made by co-expressing X0112 and X0113.

### 2.1.3 Construction of 2E12 CH2H3CH1 (X0115)

First, the CH1 fragment was cloned by PCR using the oligonucleotides CH1xbaF (SEQ ID NO:13) and CH1NotR (SEQ ID NO:14) with template X0038, a construct containing the CH1 region. The PCR fragment was subsequently isolated and digested with XbaI and NotI restriction enzymes. Second, the CH2-CH3 domain was cloned out by PCR using the oligonucleotides CH2xhoF (SEQ ID NO:15) and CH3xbaR (SEQ ID NO:16) with template M0077 containing the wild type CH2CH3. The fragment was then isolated and digested with XhoI and XbaI restriction enzymes. The first and second fragments were then ligated into the PD28 vector using the XhoI and NotI restrition sites.

The 2E12 scFv binding domain was cloned out by PCR using the oligoucleotides 2E12AgeF (SEQ ID NO:7) and 2E12XhoR (SEQ ID NO:8) with mutated M0039 as template. The fragment was isolated, digested with AgeI and XhoI and ligated into the new vector as described above utilizing the AgeI and XhoI sites.

The nucleotide sequence encoding, and the amino acid sequence of, the resulting single chain fusion protein, X0115, are set forth in SEQ ID NOS:25 and 26. The first 60 nucleotides in SEQ ID NO:25 encode a 20 amino acid signal peptide in SEQ ID NO:26.

### 2.1.4 Construction of CH2CH3Ck (X0114)

First, the Ck fragment was cloned out by PCR out using the oligonucleotides, CkxbaF (SEQ ID NO:17) and CkNotR (SEQ ID NO:18) with template X0033 containing the Ck domain. The PCR fragment was subsequently isolated and digested with xbaI and NotI restriction enzymes. Second, the CH2CH3 domain was cloned by PCR using the oligonucleotides, CH2AgeF (SEQ ID NO:19) and CH3XbaR (SEQ ID NO:20) with template M0077 containing the wild type CH2CH3. The fragment was then isolated and digested with AgeI and XbaI. The two fragments were then ligated into the PD28 vector using the AgeI and NotI sites.

The nucleotide sequence encoding, and the amino acid sequence of, the resulting single chain fusion protein, X0114, are set forth in SEQ ID NOS:27 and 28. The first 60 nucleotides in SEQ ID NO:27 encode a 20 amino acid signal peptide in

SEQ ID NO:28. X0126, an Interceptor with an amino terminal CH1-Ck heterodimerization pair, was made by co-expressing X0115 and X0114.

### 2.1.5. Construction of X0116, X0017, X0118 and X0119

X0116, X0117, X0118 and X0119 were constructed by introducing F405A and Y407A mutations into X0112, X0113, X0114 and X0115, respectively. The wild type copy of the CH3 domains from these constructs were swapped with the mutated copy of CH3 containing the two mutations from X0045, a construct containing the F405A and Y407A mutations.

The nucleotide and amino acid sequences for X0116 are set forth in SEQ ID NOS:29 and 30, for X0117 in SEQ ID NOS:31 and 32, for X0118 in SEQ ID NOS:33 and 34, and for X0119 in SEQ ID NOS:35 and 36, respectively. The first 60 nucleotides in SEQ ID NOS:29, 31, 33, and 35 encode a 20 amino acid signal peptide in SEQ ID NOS:30, 32, 34, and 36, respectively.

Interceptor X0125 with disabled CH3 interaction and CH1-Ck heterodimerization pair at the amino terminus was made by co-expressing X0116 and X0117, while Interceptor X0127 with disabled CH3 interaction and CH1-Ck heterodimerization pair at the carboxyl terminus was made by co-expressing X0118 and X0119.

### 2.1.6 Construction of 2E12 CH1CH2CH3CH1 (X0120) and CkCH2CH3Ck (X0121)

X0115 was digested with BsrGI and NotI to release the CH1 fragment. The CH1 fragment was then isolated and ligated into X0112 that had been cut with BsrGI and NotI to generate X0120 .

Similarly, X0114 was digested with BsrGI and NotI to release Ck fragment which was then isolated and ligated into X0113 that had been cut with BsrGI and NotI to generate X0121.

The nucleotide and amino acid sequences for X0120 are set forth in SEQ ID NOS:37 and 38, and for X0121 in SEQ ID NOS:39 and 40, respectively. The first 60 nucleotides in SEQ ID NO:37 encode a 20 amino acid signal peptide in SEQ ID NO:38. Co-expression of X0120 and X0121 yielded the X0128 Interceptor, which has two CH1-Ck heterodimerization domain pairs.

### 2.1.7 Construction of 2E12 CH1CH2CH3CH1 F405A Y407A (X0122) and CkCH2CH3Ck F405A Y407A (X0123)

X0122 and X0123 were created by swapping the BsrGI and NotI fragments of X0116 with X0119 and X0117 with that of X0118, respectively, as described in section 2.1.5.

The nucleotide and amino acid sequences for X0122 are set forth in SEQ ID NOS:41 and 42, and for X0123 in SEQ ID NOS:43 and 44, respectively. The first 60 nucleotides in SEQ ID NOS:41 and 43 encode a 20 amino acid signal peptide in SEQ ID NOS:42 and 44, respectively. Interceptor X0129 was made by co-expressing X0122 and X0123.

### 2.1.8 Construction of 2E12CH1CH2CH3Ck (X0130) and CkCH2CH3CH1 (X0131)

X0130 and X0131 were created by swapping the BsrGI and NotI fragment of X0120 with X0121 and X0121 with that of X0120, respectively.

The amino acid sequences for X0130 are set forth in SEQ ID NOS:45 and 46, and for X0131 in SEQ ID NOS:47 and 48, respectively. The first 60 nucleotides in SEQ ID NOS:45 and 47 encode a 20 amino acid signal peptide in SEQ ID NOS:46 and 48, respectively. The Interceptor X0132 was made by co-expressing X0130 and X0131.

### 2.1.9 Construction of CH2CH3Ck F405A (X0136), 2E12CH2CH3CH1 F405A (X0137), CH2CH3Ck Y407A (X0139) and 2E12CH2CH3CH1 Y407A (X0140)

X0136 and X0137 were constructed by introducing F405A mutation into X0114 and X0115, respectively. The wild type CH3 domains of X0114 and X0115 were swapped with the CH3 domain from X0095, a construct containing the F405A mutation.

The nucleotide and amino acid sequences for X0136 are set forth in SEQ ID NOS:53 and 54, and for X0137 in SEQ ID NOS:55 and 56, respectively. The first 60 nucleotides in SEQ ID NOS:53 and 55 encode the first 20 amino acids, a signal peptide, in SEQ ID NOS:54 and 56, respectively.

X0139 and X0140 were constructed by introducing Y407A mutation into X0114 and X0115, respectively. Again, the CH3 domains from X0114 and X0115 were swapped with the CH3 domain of X0096, a construct containing the Y407A mutation.

The nucleotide and amino acid sequences for X0139 are set forth in SEQ ID NOS:57 and 58, and for X0140 in SEQ ID NOS:59 and 60, respectively. The first 60 nucleotides in SEQ ID NOS:57 and 59 encode a 20 amino acid signal peptide in SEQ ID NOS:58 and 60, respectively.

Interceptor X0138 with partially disabled CH3 interactions was made by co-expressing X0136 and X0137, whereas Interceptor X0141 was made by co-expressing X0139 and X0140.

### 2.1.10 Construction of CλCH2CH3 (X0133) and CH2CH3Cλ (X0134)

Cλ was cloned from tonsil cDNA (Clonetech) and engineered with the XbaI and NotI sites. The isolated fragment was then ligated into X0113 and X0114 to give X0133 and X0134, respectively.

The nucleotide and amino acid sequences for X0133 are set forth in SEQ ID NOS:49 and 50, and for X0134 in SEQ ID NOS:51 and 52, respectively. The first 60 nucleotides in SEQ ID NOS:49 and 51 encode a 20 amino acid signal peptide in SEQ ID NOS:50 and 52, respectively.

Interceptor X0142 with CH1-Cλ heterodimerization domain pair at the front end was created by co-expressing X0115 and X0133. Interceptor X0143 with CH1-Cλ heterodimerization domain pair at the back end was created by co-expressing X0115 and X0134.

### 2.1.11 Construction of 2E12CH1CH2CH3Cλ (X0146), CλCH2CH3CH1 (X0147) and CλCH2CH3Cλ (X0148)

X0146 (2E12CH1CH2CH3Cλ) was made by replacing BsrGI/NotI fragment of X0130 (2E12CH1CH2CH3Cκ) with the same fragment from CH2CH3Cλ (X0134). X0147 was made by replacing HindIII/BsrGI fragment of X0131 with the same fragment from CλCH2CH3 (X0133). X0148 was made by replacing HindIII/BsrGI fragment of CH2CH3Cλ (X0134) with the same fragment from CλCH2CH3 (X0133).

The nucleotide and amino acid sequences for X0146 are set forth in SEQ ID NOS:61 and 62, for X0147 in SEQ ID NOS:63 and 64, and for X0148 in SEQ ID NOS:65 and 66, respectively. The first 60 nucleotides in SEQ ID NOS:61 and 63 encode a 20 amino acid signal peptide in SEQ ID NOS:62 and 64, respectively.

### 2.1.12 Construction of CH1CH2CH3Ck (X0167) and CkCH2CH3CH1 2E12 (X0168)

X0167 was made by removing the 2E12 scFv fragment from X0130 (2E12CH1CH2CH3Cκ) using PCR. Briefly, the CH1CH2 fragment was cloned by PCR and reinserted into the same X0130 vector that had been cut with AgeI and BsrGI site, thereby deleting the 2E12 scFv sequence. For X0168, X0131 (CKCH2CH3CH1) was used as a template in which 2E 12 scFv was added on the carboxyl terminus of the molecule. An NKG2D linker (SEQ ID NO:124) was used to link the C-terminus of CH1 with the N-terminus of 2E12 scFv.

The nucleotide and amino acid sequences for X0167 are set forth in SEQ ID NOS:67 and 68, and for X0168 in SEQ ID NOS:69 and 70, respectively. The first 60 nucleotides in SEQ ID NOS:67 and 69 encode a 20 amino acid signal peptide in SEQ ID NOS:68 and 70, respectively. Interceptor X0171 was created by co-expressing X0167 and X0168 whereas Interceptor X0172 was created by co-expressing X0130 and X0168.

### 2.1.13 Altering Cκ Amino Acids

### 2.1.13.1 Mutagenesis of X0113

The Cκ domain of X0113 without a signal peptide (SEQ ID NO:71) was mutated with the Invitrogen Quikchange kit at positions N29, N30, Q52, V55, T56, S68 and T70. These residues are also known as N137, N138, Q160, V163, T164, S176 and T178, respectively, from the PDB database (PDB entry #1B6D). Each position was mutated to an alanine (A), resulting in the following versions of X0113: N29A, N30A, Q52A, V55A, T56A, S68A and T70A. The amino acid sequences of X0113 N29A, X0113 N30A, X0113 Q52A, X0113 V55A, X0113 T56A, X0113 S68A, and X0113 T70A are set forth in SEQ ID NOS:72-78, respectively. Co-expressing of X0112 and the X0113 variants resulted in the creation of various Interceptors: X0124 N29A, X0124 N30A, X0124 Q52A, X0124 V55A, X0124 T56A, X0124 S68A and X0124 T70A.

Double and triple alanine mutations of X0113 were also made as listed below:

| | |
|---|---|
| X0113 V55A N29A | (SEQ ID NO: 79) |
| X0113 V55A N30A | (SEQ ID NO: 80) |
| X0113 V55A Q52A | (SEQ ID NO: 81) |
| X0113 V55A T56A | (SEQ ID NO: 82) |
| X0113 V55A S68A | (SEQ ID NO: 83) |
| X0113 V55A T70A | (SEQ ID NO: 84) |
| X0113 V55A N29A N30A | (SEQ ID NO: 85) |
| X0113 V55A N29A S68A | (SEQ ID NO: 86) |
| X0113 V55A S68A T70A | (SEQ ID NO: 87) |
| X0113 V55A Q52A S68A | (SEQ ID NO: 88) |

These were co-transfected with X0112 to generate the respective mutants of X0124.

### 2.1.13.2 Mutations on Selected Residues to Bulky Side Chain Amino Acids (R,W,Y,E,Q,L)

Other mutations were made on four interface residues Q52, T56, S68 and T70 as follows: X0113 Q52R (SEQ ID NO:117), X0113 Q52W (SEQ ID NO:118), X0113 T56R (SEQ ID NO:119), X0113 T56W (SEQ ID NO:120), X0113 S68R (SEQ ID NO:121), X0113 S68W (SEQ ID NO:122), X0113 T7OR (SEQ ID NO:123), and X0113 T70W (SEQ ID NO:124). In addition, a combination of bulky amino acid side chain mutations and alanine mutations were made as shown:

| | |
|---|---|
| X0113 N29R V55A T70A | (SEQ ID NO: 89) |
| X0113 N29K V55A T70A | (SEQ ID NO: 90) |
| X0113 N29W V55A T70A | (SEQ ID NO: 91) |
| X0113 N29Y V55A T70A | (SEQ ID NO: 92) |
| X0113 S68K V55A | (SEQ ID NO: 93) |
| X0113 S68E V55A | (SEQ ID NO: 94) |
| X0113 S68Q V55A | (SEQ ID NO: 95) |
| X0113 T70E V55A | (SEQ ID NO: 96) |
| X0113 Q52L N29A N30A | (SEQ ID NO: 97) |
| X0113 N30R V55A T70A | (SEQ ID NO: 98) |
| X0113 N30K V55A T70A | (SEQ ID NO: 99) |
| X0113 N30W V55A T70A | (SEQ ID NO: 100) |
| X0113 N30E V55A T70A | (SEQ ID NO: 101) |
| X0113 N30G V55A T70A | (SEQ ID NO: 102) |
| (not a bulky side | |
| chain mutation) | |
| X0113 V55R N29A N30A | (SEQ ID NO: 103) |
| X0113 V55W N29A N30A | (SEQ ID NO: 104) |
| X0113 V55E N29A N30A | (SEQ ID NO: 105) |

All these mutated X0113 were co-transfected with X0112 to generate X0124 with the respective mutations.

### 2.2 Expression of Various Constructs in HEK293 Cells

The day before transfection, HEK292 cells were suspended at a cell concentration of 0.5×10⁶ cells/ml in Freestyle 293 expression medium (Gibco). For a large transfection, 250 ml of cells were used, but for a small transfection, 60 ml of cells were used. On the transfection day, 320 ul of 293fectin reagent (Invitrogen) was mixed with 8 ml of media. At the same time, 250 ug of DNA for each of the two chains were also mixed with 8ml of media and incubated for 5 minutes. In some transfections, a ratio of 2:1 long to short chains were used. In such a case, 250 ug of long chain DNA and 125 ug of short chain DNA were used. The medium with the 293fectin was then added to the medium with the DNA. After 15 minutes of incubation, the DNA-293fectin mixture was added to the 250m1 of 293 cells and returned to the shaker at 37° C. and shaken at a speed of 120 RPM. For the smaller transfection using 60 ml of cells, a fourth of the DNA, 293fectin and media were used. Table 3 is the list of co-transfections that were performed:

TABLE 3
Exemplary Interceptors

| Interceptor | Chain 1 ID (long chain | Chain 2 ID (short chain |
|---|---|---|
| ID | containing binding domain) | with no binding domain) |
| X0124 | X0112 (SEQ ID NO: 22) | X0113 (SEQ ID NO: 24) |
| X0125 | X0116 (SEQ ID NO: 30) | X0117 (SEQ ID NO: 32) |
| X0126 | X0115 (SEQ ID NO: 26) | X0114 (SEQ ID NO: 28) |
| X0127 | X0119 (SEQ ID NO: 36) | X0118 (SEQ ID NO: 34) |
| X0128 | X0120 (SEQ ID NO: 38) | X0121 (SEQ ID NO: 40) |
| X0129 | X0122 (SEQ ID NO: 42) | X0123 (SEQ ID NO: 44) |
| X0132 | X0130 (SEQ ID NO: 46) | X0131 (SEQ ID NO: 48) |
| X0138 | X0137 (SEQ ID NO: 56) | X0136 (SEQ ID NO: 54) |
| X0141 | X0140 (SEQ ID NO: 60) | X0139 (SEQ ID NO: 58) |
| X0142 | X0112 (SEQ ID NO: 22) | X0133 (SEQ ID NO: 50) |
| X0143 | X0115 (SEQ ID NO: 26) | X0134 (SEQ ID NO: 52) |
| X0149 | X0146 (SEQ ID NO: 62) | X0147 (SEQ ID NO: 64) |
| X0150 | X0120 (SEQ ID NO: 38) | X0148 (SEQ ID NO: 66) |
| X0165 | X0130 (SEQ ID NO: 46) | X0147 (SEQ ID NO: 64) |
| X0166 | X0146 (SEQ ID NO: 62) | X0131 (SEQ ID NO: 48) |
| X0171 | X0168 (SEQ ID NO: 70) | X0167 (SEQ ID NO: 68) |

Transfections were performed at the 60 ml scale for the mutated Interceptors where X0112 and mutated X0113 were co-transfected at a ratio of 2:1. Below is the list of X0113 mutants that were co-transfected with X0112:

Set A: N29A, N30A, Q52A, V55A, T56A, S68A and T70A (SEQ ID NOS:72-78, respectively)

Set B: V55A N29A, V55A N30A, V55A Q52A, V55A T56A, V55A S68A, V55A T70A, V55A N29A N30A, V55A N29A S68A, V55A S68A T70A, V55A Q52A S68A (SEQ ID NOS:79-88, respectively)

Set C: Q52R, Q52W, T56R, T56W, S68R, S68W, T70R, T70W (SEQ ID NOS:117-124, respectively)

Set D: N29R V55A T70A, N29K V55A T70A, N29W V55A T70A, N29Y V55A T70A, S68K V55A, S68E V55A, S68Q V55A, T70E V55A, Q52L N29A N30A, N30R V55A T70A, N30K V55A T70A, N30W V55A T70A, N30E V55A T70A, N30G V55A T70A, V55R N29A N30A, V55W N29A N30A, V55E N29A N30A (SEQ ID NOS:89-105, respectively)

Additional Interceptors were made as shown Table 4 below:

TABLE 4
Additional Exemplary Interceptors

| | Interceptor | Chain 1 ID (long chain | Chain 2 ID (short chain |
|---|---|---|---|
| Interceptor Characteristics | ID | containing binding domain) | with no binding domain) |
| 2E12 scFv is the binding | X0124 | X0112 (SEQ ID NO: 22) | X0113 (SEQ ID NO: 24) |
| domain in Chain 1 | | | |
| Ck near N-terminus of Chain 2 | | | |
| Ck of chain 2 contains WYAE | X0232 | X0112 (SEQ ID NO: 22) | X0229 (SEQ ID NO: 127) |
| (N29W N30Y V55A T70E) | | | |
| mutations | | | |
| Ck of chain 2 contains YYAE | X0233 | X0112 (SEQ ID NO: 22) | X0231 (SEQ ID NO: 129) |
| (N29Y N30Y V55A T70E) | | | |
| mutations | | | |
| Ck of chain 2 contains EAE | X0211 | X0112 (SEQ ID NO: 22) | X0193 (SEQ ID NO: 131) |
| (N30E V55A T70E) mutations | | | |
| Ck of chain 2 contains YAE | X0224 | X0112 (SEQ ID NO: 22) | X0220 (SEQ ID NO: 133) |
| (N30Y V55A T70E) mutations | | | |
| P2C2 scFv is the binding | X0235 | X0234 (SEQ ID NO: 135) | X0113 (SEQ ID NO: 24) |
| domain in Chain 1 | | | |
| Ck is near N-terminus of Chain 2 | | | |
| Ck of chain 2 contains YAE | X0236 | X0234 (SEQ ID NO: 135) | X0220 (SEQ ID NO: 133) |
| (N30Y V55A T70E) mutations | | | |
| Ck of chain 2 contains EAE | X0237 | X0234 (SEQ ID NO: 135) | X0193 (SEQ ID NO: 131) |
| (N30E V55A T70E) mutations | | | |
| 2E12 scFv is the binding | X0126 | X0115 (SEQ ID NO: 26) | X0114 (SEQ ID NO: 28) |
| domain in Chain 1 | | | |
| Ck is near C-terminus of Chain 2 | | | |
| Ck of chain 2 contains YAE | X0238 | X0115 (SEQ ID NO: 26) | X0225 (SEQ ID NO: 137) |
| (N30T V55A T70E) substitutions | | | |

### 2.3 Protein Purification

Protein A affinity chromatography was used to purify all the proteins. 2 mL of packed protein A agarose (Repligen) was added to a Biorad column (Econo-column chromatography column, size 2.5×10 cm), washed extensively with PBS (10× column volume) and the supernatants were loaded, washed with PBS again and eluted with 3 column volume of Pierce IgG elution buffer. Proteins were then dialyzed extensively against PBS. Proteins were then concentrated using Amicon centrifugal filter devices to a final volume of around 0.5 mL.

For second step purification, Protein L affinity chromatography or cation exchange chromatography were used. For Protein L purification, protein A purified Interceptor was passed over a Protein L agarose column that had been pre-equilibrated with PBS, washed with PBS (10× column volume) and then eluted with Pierce IgG elution buffer. Proteins were then dialyzed against PBS extensively and concentrated using Amicon centrifugal filter devices to a final volume of around 0.5 mL.

Samples (200-300 ug) of previously affinity purified (Protein A or Protein L) Interceptor constructs were dialyzed into 20 mM MES, pH 6.0 (Buffer A) and loaded onto a MonoS 5/50 GL cation exchange column (GE Healthcare) at a flow rate of 2 mL/min, using an AKTA Explorer FPLC. The column was allowed to equilibrate for 5 column volumes (CV) and then run in a gradient format to a mixture of 50%:50% buffer A:buffer B (buffer B being 20 mM MES, 1 M NaCl, pH 6.0) over 20 CV. A following mixture of 100% buffer B was run for 5 CV to clean the column, and the system was run for another 5 CV at 100% buffer A to re-equilibrate prior to the next injection. Peaks were collected and analyzed by SDS-PAGE and electrospray mass spectrometry.

### 2.4 Physical Characterization of Proteins on SDS-PAGE and HPLC Size Exclusion Column

All proteins purified were analyzed on a 10% SDS-PAGE gel using Invitrogen's X-cell Surelock gel box. Size exclusion chromatography was performed on an AKTA Explorer FPLC (Pharmacia Biotech) using a Superdex200 10/300 GL column. Some proteins were analyzed by electrospray mass spectrometry using an Agilent 6120 TOF ES/MS.

### 2.5 ELISA Assay for Interceptor Binding

One ug of CD28 mIg was coated overnight on a FluoroNunc Maxisorp Maxisorp ELISA plate (Nunc). The plate was washed two times with PBS containing 0.1% Tween 20 and blocked with 5% non-fat milk in PBS for 1 hour. The plate was then washed twice with PBS containing 0.1% Tween 20. Varying concentrations of

Interceptors were added to each well. The plate was then incubated for a further 1 hour and washed 4 times with PBS containing 0.1% Tween 20. 100 ul of 1000× diluted anti-huIgG HRP (Jackson Immunolabs) or anti-huk HRP (Southern Biotechnology) was added and incubated for 1 hour. The plate was washed 3 times with 5% non-fat milk in PBS and Quantablue NS/K Fluorogenic substrate (Pierce) was added. After 5 minutes of incubation, the plate was read on a Spectra Max Gemini XS plate reader (Molecular Devices). The samples were excited at 325 nm and emission at 420 nm was monitored. Results were expressed as fluorescence units versus concentration of Interceptors.

### 2.6 Thymidine Incorporation Assays

2.6.1 Synergy with Suboptimal Concentration of PMA

Peripheral blood mononuclear cells (PBMC) from in-house donors were isolated from heparinized whole blood via centrifugation over Lymphocyte Separation Media (MP Biomedicals, Aurora, Ohio) and washed two times with RPMI media (Gibco-Invitrogen, Carlsbad, Calif.). CD4+ T-cells were then enriched from the PBMC using negative selection with a MACS CD4+ T-cell Isolation Kit (Miltenyi Biotec, Auburn, Calif.). The enriched (>95%) CD4+ T-cells were then resuspended at a concentration of 1×10⁶ cell/ml in complete RPMI/10% FCS. Test reagents were prepared at 40 ug/ml (yielding a final concentration of 10 ug/ml) in complete RPMI/10% FCS and added in 50 un/well to flat-bottom 96-well plates (BD Falcon, San Jose, Calif.). PMA (Phorbol 12 myristate 13-acetate; A.G. Scientific, Inc., San Diego, Calif.) in complete RPMI/10% FCS was added in 50 ul/well at 4 ng/ml (final concentration of 1 ng/ml). Then T-cells in complete RPMI/10% FCS were added at a concentration of 5×10⁴ cells/well in a 50 ul volume, and finally an appropriate amount of complete RPMI/10% FCS was added to each well (typically 50 ul) to bring the final volume to 200 ul/well. The cells were treated with the test samples +/−PMA and incubated for 72 hours at 37° C. in 5% CO₂. One microliter of tritiated thymidine (Amersham Biosciences, Pisctaway, N.J.) in a 1:50 dilution of complete RPMI/10% FCS (50 ul/well) was added to the wells for the last 6 hours of culture. Plates were harvested onto a Unifilter-96, GF/C microplate (Perkin Elmer, Boston, Mass.) with a Packard Filtermate Harvester (Perkin Elmer, Boston, Mass.). Numbers are expressed as cpm and are the mean of replicate samples.

### 2.6.2. MLR Blocking Assay (Primary)

Peripheral blood mononuclear cells (PBMC) were isolated from heparinized whole blood via centrifugation over Lymphocyte Separation Media (MP Biomedicals, Aurora, Ohio), washed two times with RPMI media (Gibco-Invitrogen, Carlsbad, Calif.) and then resuspended in complete RPMI/10% FCS at a concentration of 8×10⁵/ml. WIL2-S (Manassas, Va.), a B-cell lymphoblast line, was treated with mitomycin C (Sigma Alderich, St. Louis, Mo.) to inhibit proliferation. Briefly, WIL2-S cells were resuspended in complete RPMI/10%FCS at a concentration of 5×10⁶ cells/ml and mitomycin C was added at 40 ug/ml. The cells were incubated for 40 minutes in a 37° C. water bath,then washed 3 times in complete media and resuspended at 2×10⁵ cells/ml in complete RPMI/10% FCS. Test reagents were prepared at 40 ug/ml (10 ug/ml final concentration) in complete RPMI/10% FCS and added in 50 ul/well to a 96-well flat bottom tissue culture plate (BD Falcon, San Jose, Calif.). The PBMC were then added to each well followed by the mitomycin C treated WIL2-S, and an appropriate amount of complete RPMI/10% FCS was added to each well to bring the final volume to 200 ul/well. The PBMC were tested with the test samples +/−WIL2-S and cultured for 96 hours at 37° C. in 5% CO₂. One microliter of tritiated thymidine (Amersham Biosciences, Pisctaway, N.J.) in a 1:50 dilution of complete RPMI/10% FCS (50 ul/well) was added to the wells for the last 10 hours of culture. Plates were harvested onto a Unifilter-96, GF/C microplate (Perkin Elmer, Boston, Mass.) with a Packard Filtermate Harvester (Perkin Elmer, Boston, Mass.). Numbers are expressed as cpm and are the mean of replicate samples.

### 2.6.3. MLR Blocking Assay (Secondary)

Peripheral blood mononuclear cells (PBMC) were isolated from heparinized whole blood via centrifugation over Lymphocyte Separation Media (MP Biomedicals, Aurora, Ohio), washed two times with RPMI media (Gibco-Invitrogen, Carlsbad, Calif.) and then resuspended in complete RPMI/10% FCS at a concentration of 1×10⁶/ml in a tissue culture flask. WIL2-S (Manassas, Va.), a B-cell lymphoblast line, was treated with mitomycin C (Sigma Alderich, St. Louis, Mo.) to inhibit proliferation. Briefly, WIL2-S cells were resuspended in complete RPMI/10%FCS at a concentration of 5×10⁶ cells/ml and mitomycin C was added at 40 ug/ml. The cells were incubated for 40 minutes in a 37° C. water bath, then washed 3 times in complete media and added to the isolated PBMC in the tissue culture flask at a ratio of 1:4 WIL2-S:PBMC. After one week, the primary blasts were harvested and washed twice in RPMI/10% FCS. They were then resuspended in RPMI/10% FCS at a concentration of 8×10⁵/ml. WIL2-S cells were isolated and treated with mitomycin-C as in the primary stimulation and then resuspended in RPMI/10% FCS at a concentration of 2×10⁵/ml. Test reagents were prepared at 40 ug/ml (10 ug/ml final concentration) in complete RPMI/10% FCS and added in 50u1 to a 96-well flat bottom tissue culture plate (BD Falcon, San Jose, Calif.). Appropriate amounts of complete RPMI/10% FCS were added to wells to bring the final volume (after addition of the PBMC and mitomycin C treated WIL2-S) of the wells to 200 ul. The blasts were then added to the wells in 50 ul and finally the WIL2-S in 50 ul. The blasts were tested with the test samples +/−WIL2-S and incubated for 96 hours at 37° C. in 5% CO₂. One microliter of tritiated thymidine (Amersham Biosciences, Pisctaway, N.J.) in a 1:50 dilution of complete RPMI/10% FCS (50 ul/well) was added to the wells for the last 10 hours of culture. Plates were harvested onto a Unifilter-96, GF/C microplate (Perkin Elmer, Boston, Mass.) with a Packard Filtermate Harvester (Perkin Elmer, Boston, Mass.). Numbers are expressed as cpm and are the mean of replicate samples.

### 2.7 FACS Staining Assay

Peripheral blood mononuclear cells (PBMC) were isolated from heparinized whole blood via centrifugation over Lymphocyte Separation Media (MP Biomedicals, Aurora, Ohio) and washed two times with RPMI media (Gibco-Invitrogen, Carlsbad, Calif.). CD3+ T-cells were then enriched from the PBMC using negative selection with a MACS CD3+ T-cell Isolation Kit (Miltenyi Biotec, Auburn, Calif.). The enriched (>95% CD3+ T-cells) CD3+ T-cells were then resuspended at a concentration of 4×10⁶ cell/ml in staining media, PBS (Gibco-Invitrogen) with 2% goat serum (Gemini Bioproducts, Woodland, Calif.). The test reagents were serially diluted two-fold in staining media beginning at 20 ug/ml (twice the final concentration). The test samples were plated in a 96-well “V” bottom plate (BD Falcon, San Jose, Calif.) and the enriched T-cells were then added to the wells. The control of staining media alone was also plated. The cells were incubated for 45 minutes on ice and then washed with PBS. The cells were then resuspended in 50 ul of a 1:100 dilution of PE conjugated F′2 Goat anti-Human IgG (Jackson Immunoresearch Laboratories, West Grove, Pa.) in staining media. The control of PE conjugated F′2 Goat anti-Human Ig was also added. The cells were incubated for 30 minutes in the dark on ice. They were then washed with cold PBS and resuspended in PBS with 0.1% paraformaldehyde (USB Corp, Cleveland, Ohio). The cells were then run on a FACsCalibur Flow Cytometer and analyzed with Cell Quest software (Becton Dickinson, San Jose, Calif.).

### 2.8 Surface Plasmon Resonance Analysis

Surface plasmon resonance (SPR) measurements were performed on a Biacore T100 SPR using HBS-P+ (GE Healthcare) as a running buffer. CD28-mIgG (25 μg/mL in 10 mM sodium acetate, pH 4.0) was directly immobilized onto a CM5 chip using standard amine coupling chemistry (Biacore Amine Coupling Kit, GE Healthcare), with final immobilization levels between 800 and 1900 Ru (resonance units). 2E12 binding domain constructs were injected at 25° C. or 37° C. for 150 seconds at a flow rate of 30 μl/min in a series of concentrations from 10 nM to 1 μM. Dissociation was monitored for 1200 seconds, and the surface was regenerated by injecting 50 mM NaOH for 60 seconds. Binding interactions with the surface were stable through at least 60 regeneration cycles. Data were analyzed using BiaEvaluation for the T100 software (version 2.0, GE Healthcare).

### 3. Results

### 3.1 Class 1 Interceptors

3.1.1 Class 1 Interceptor with one N-terminal Ck-CH1 Heterodimerization Domain

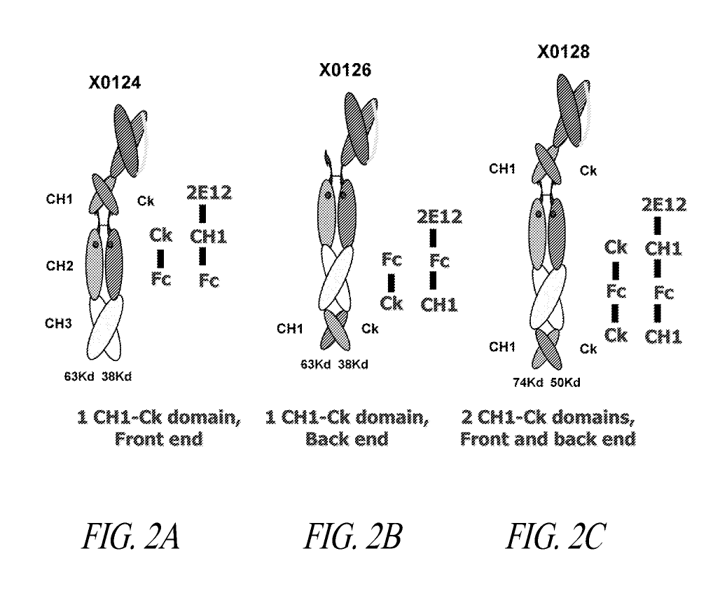

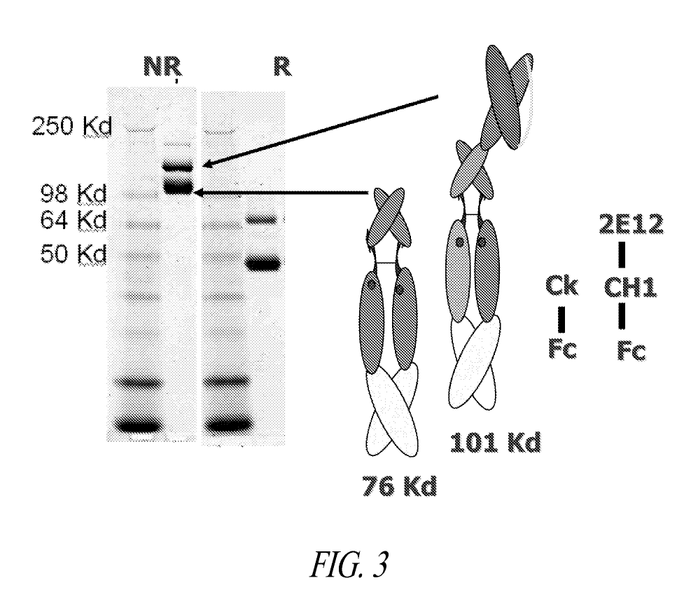

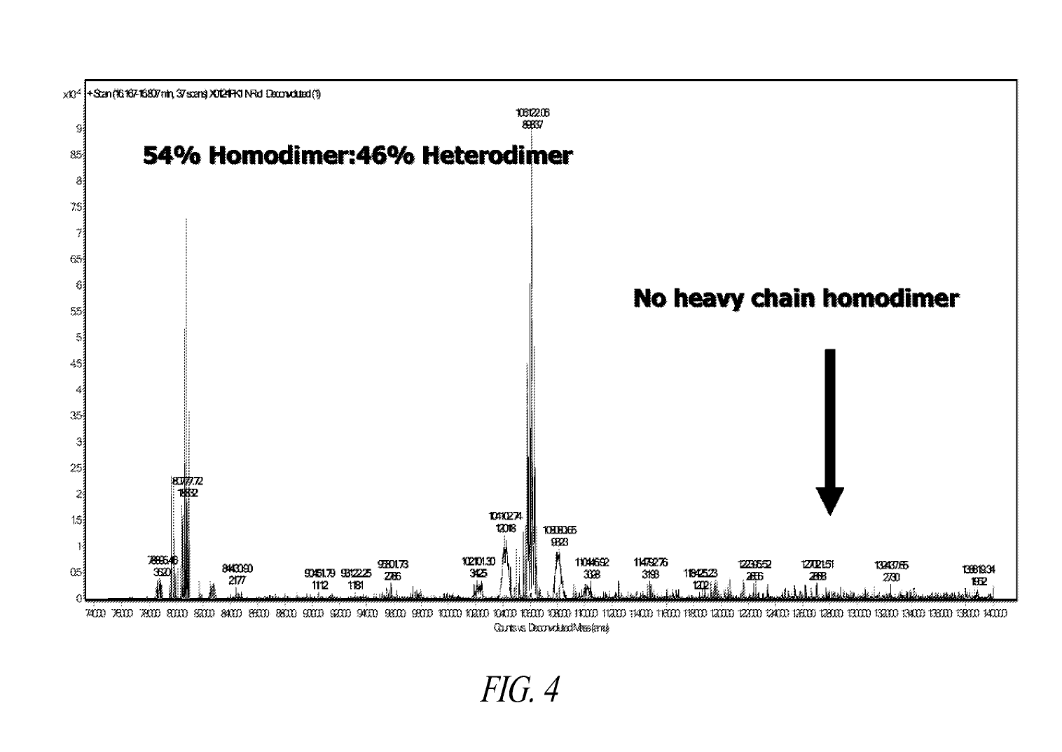

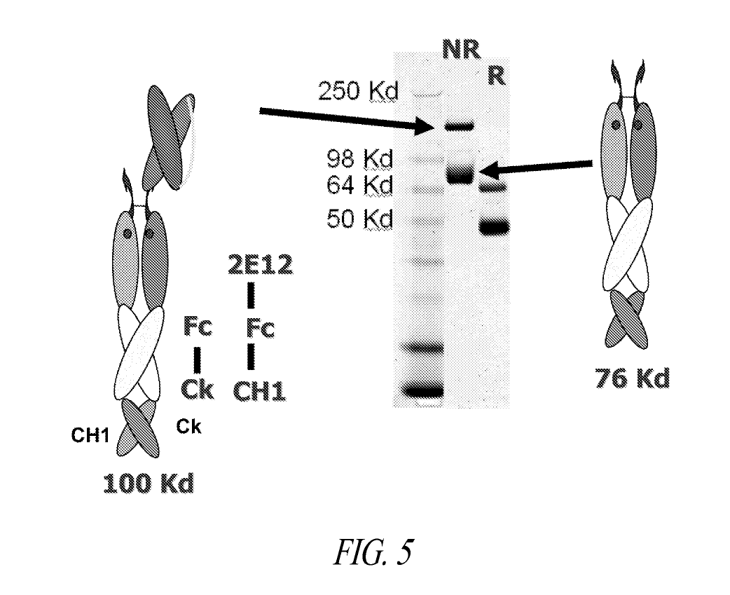

Three exemplary ways of making class 1 Interceptors are shown in FIGS. 2A, 2B and 2C. X0124, X0126 and X0128 are three examples of class 1 Interceptors. To make these Interceptors, two different DNA vectors, the long chain consisting of the binding domain and the short chain with no binding domain, were co-transfected.

The X0124 Interceptor, a class 1 Interceptor with a Ck-CH1 heterodimerization domain at the amino terminus of the molecule, was made by co-transfecting X0112 and X0013. When the long chain X0112 was transfected alone, no protein was detected. Unlike the long chain, the short chain X0113 expressed well as a homodimer when transfected alone. When the long and short chains were co-transfected, a mixture of heterodimer (Interceptor) and homodimer were assembled (FIG. 3). However, the homodimer of the heavy chain was visibly absent which can be attributed to the instability of this molecule when two CH1 regions were brought together by dimerization. This was confirmed by Mass Spectrometry analysis (FIG. 4). The absence of the long chain homodimer means that the protein mixture did not contain bivalent long chain homodimers but did contain the monovalent polypeptide heterodimer.

3.1.2 Class 1 Interceptor with one C-terminal Cκ-CH1 Heterodimerization Domain

X0126 is a class 1 Interceptor, which was made by co-expressing X0114 and X0115. As with X0124, two predominant species were seen on the SDS-PAGE gel: the heterodimer (Interceptor) and the homodimer of the short chain (FIG. 5). Again, the homodimer of the long chain was visibly absence. This experiment shows that a class 1 Interceptor may be made by placing a Cκ-CH1 heterodimerization pair at the C-terminus of a single chain fusion polypeptide.

3.1.3 Class 1 Interceptor with 2 pair of Cκ-CH1 Heterodimerization Domains.

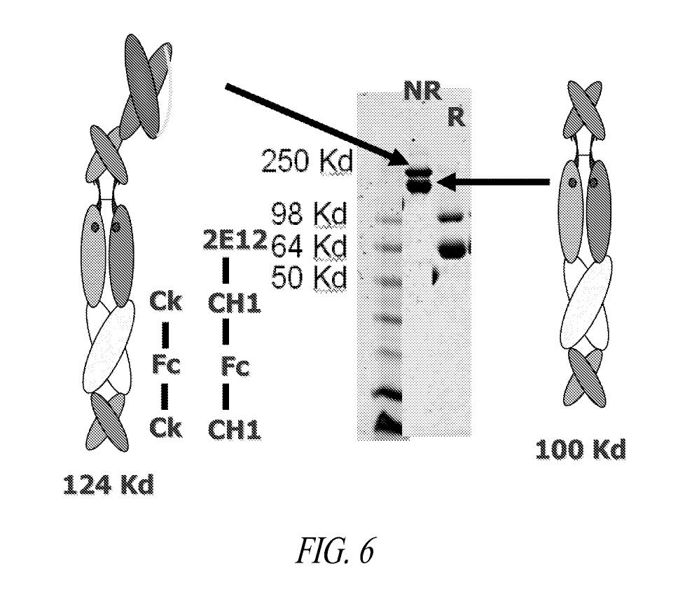

X0128 is a class I Interceptor with two Ck-CH1 pairs, one at the amino terminus of the Fc tail and another at the carboxyl terminus of the Fc tail. X0128 was made by co-expressing X0120 (comprising two CH1 regions) and X0121 (that comprising two Cκ domains). As with X0124 and X0126, the co-expression resulted in the formation of the heterodimer (Interceptor) and the homodimer of the short chain (FIG. 6). This experiment shows that a class 1 Interceptor may have a Cκ-CH1 heterodimerization pair at each end of a single chain fusion polypeptide.

3.1.4 Class 1 Interceptor with Mutations in the CH3 Domain

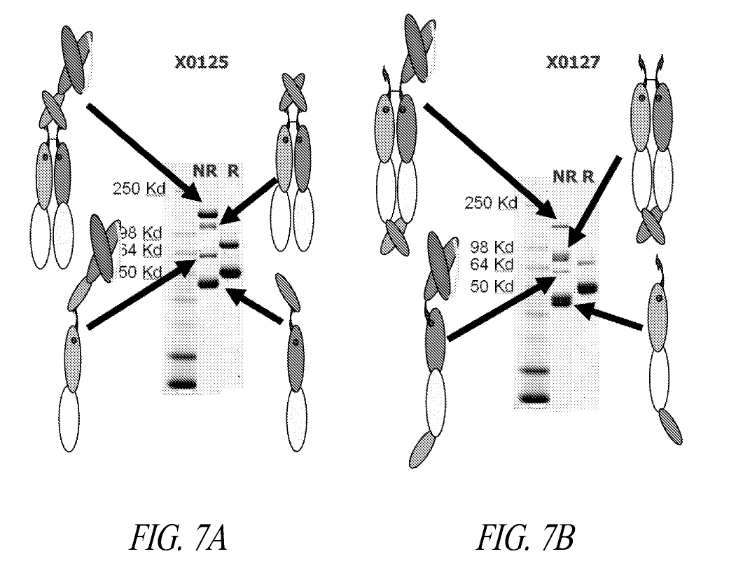

The effect of the interaction between the CH3 domains from different single chain fusion polypeptides on heterodimerization were examined by introducing two point mutations in the CH3 domain (F405A, Y407A) to disable CH3 interaction. Two Interceptors, X0125 and X0127, with these two point mutations were constructed. X0125 is similar to X0126 in having one CH1-Ck pair at the amino terminus, but is different in having the two CH3 mutations. X0127 is similar to X0126 in having one CH1-Ck pair in the carboxyl terminus, but is different in having the two CH3 mutations. As seen in FIGS. 7A and 7B, introducing these mutations in the CH3 domain destabilized both X0125 and X0127 molecules, resulting in a mixture of monomer of the long chain and short chain in addition to the homodimer of the short chain and the heterodimer.

X0129 was also made by co-expressing X0122 and X0123, which is a molecule similar to X0128 in that it has two CH1-Ck pairs except that it also carries the double mutations in the CH3 domain. Results similar to those with X0125 and X0127 were obtained: Both monomers of the short and long chain were formed (data not shown).

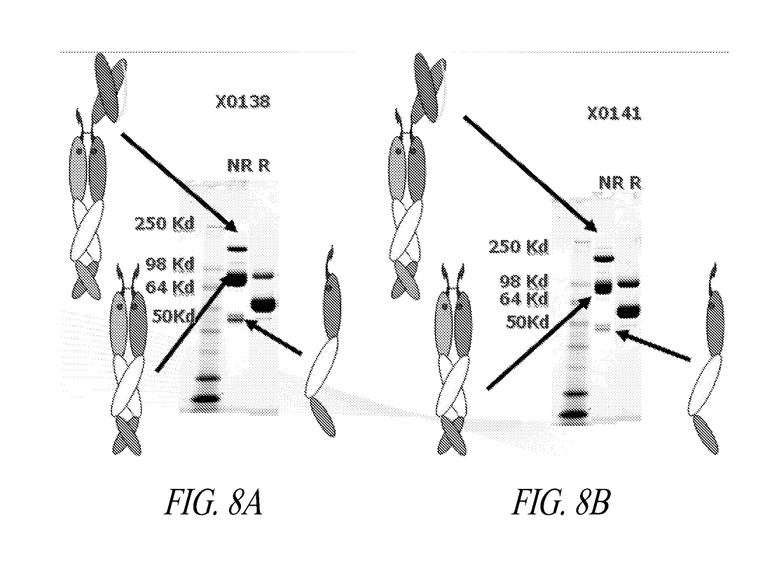

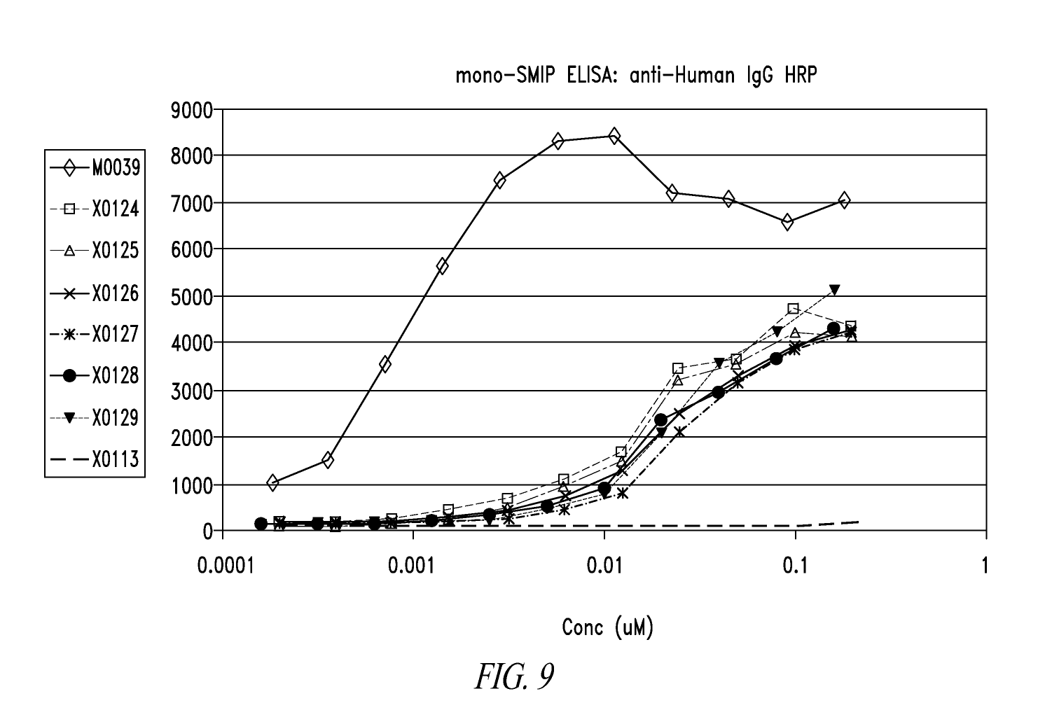

The effect of a single point mutation in CH3 (F405A or Y407A) on heterodimerization was also studied. X0138 was made by co-expressing X0136 and X0137. Both X0136 and X0137 has the single F045A mutation. As shown in FIG. 8A, monomer of the short chain seemed to be present in the protein mixture. Likewise, X0141 was made by co-expressing X0139 and X0140, both of which has the single Y407A mutation. Again, monomer of the short chain was seen in the protein sample (FIG. 8B).

Thus, it appears that the CH3 domain contributes but is not essential for the formation the polypeptide heterodimers of this disclosure.

### 3.1.5 ELISA Assay for Interceptors Binding to CD28 mIg

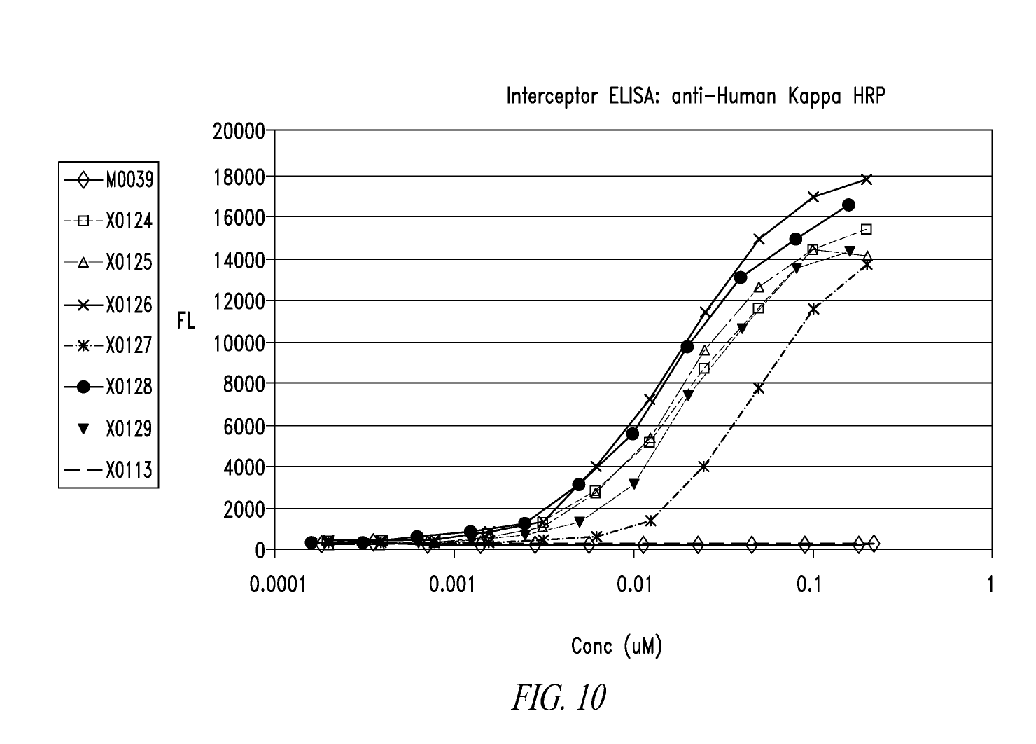

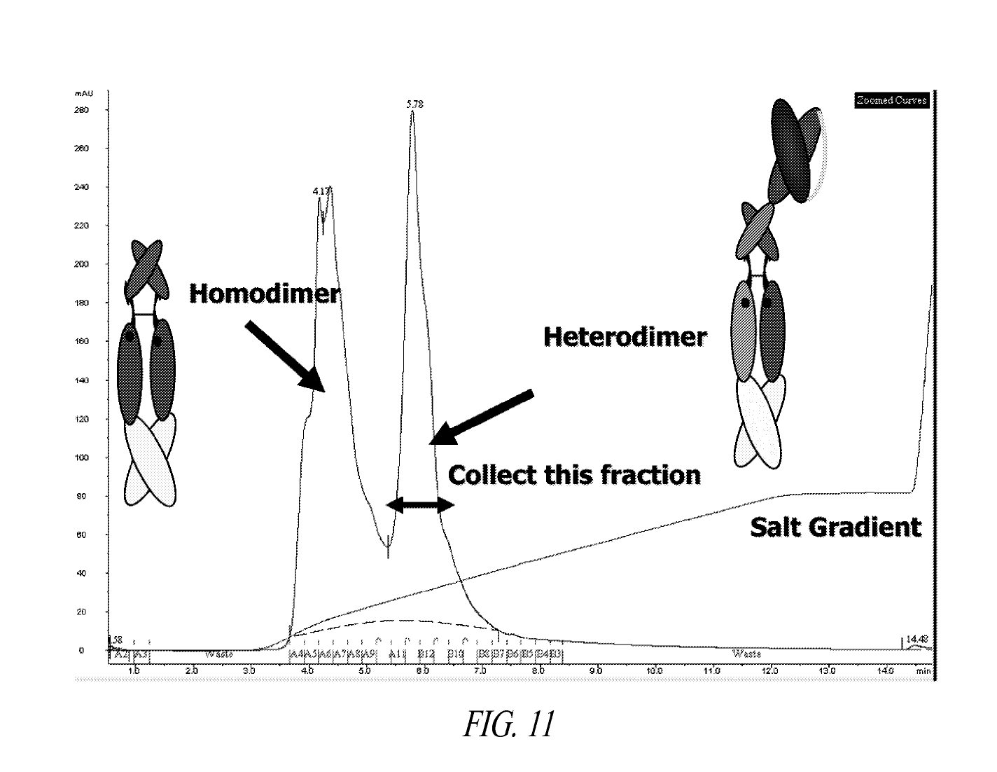

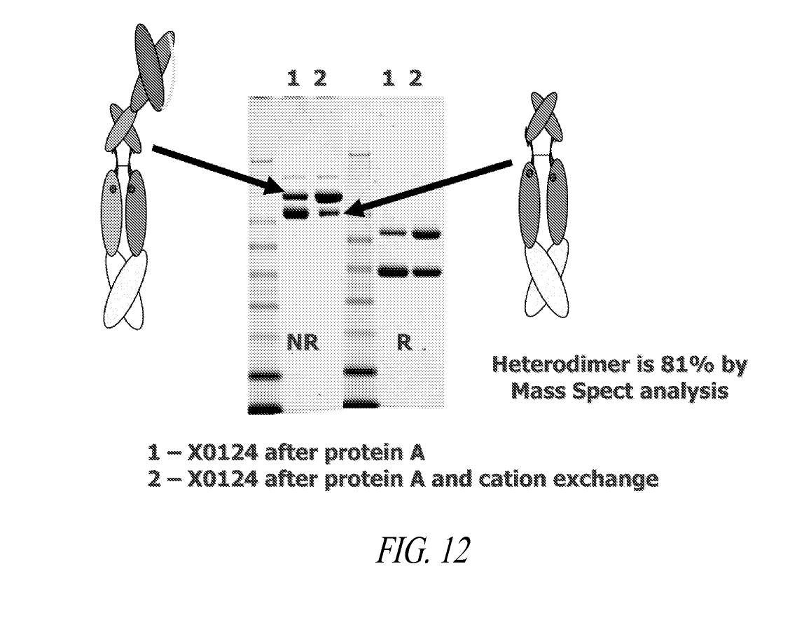

Various Interceptors containing 2E12 scFv binding domain specific for CD28 (X0124, X0125, X0126, X0127, X0128 and X0129) were tested in an ELISA by adding the proteins to CD28mIg coated plates and detecting binding with either anti-human IgG HRP conjugates or with anti-human Cκ HRP conjugates. As shown in FIG. 9, all the Interceptors bound to the specific CD28 target. The control molecule, X0113, the homodimer of the short chain with no binding domain, did not bind, whereas the bivalent SMIP protein form of 2E12 bound strongly as expected. When detected using anti-human Cκ HRP conjugates (FIG. 10), all the Interceptors binding to CD28mIg were detected since they all have the Cκ domain, but the bivalent SMIP protein, M0039, was not detected due to the absence of the Cκ domain.

### 3.1.6 Second Step Purification of Class 1 Interceptors

As a second step purification, a cation exchange column (MonoS) was used to separate the heterodimer from the homodimer. Two peaks eluted from the column following a salt gradient treatment after loading with X0124 (FIG. 11). Fractions containing the 2 peaks were examined by SDS-PAGE. The Interceptor molecule seemed to present in the second peak. FIG. 12 is the SDS-PAGE analysis of X0124 before the column purification and X0124 peak 2. It seemed that the X0124 heterodimer was enriched to about 80% and this result was verified by mass spectrometry analysis.

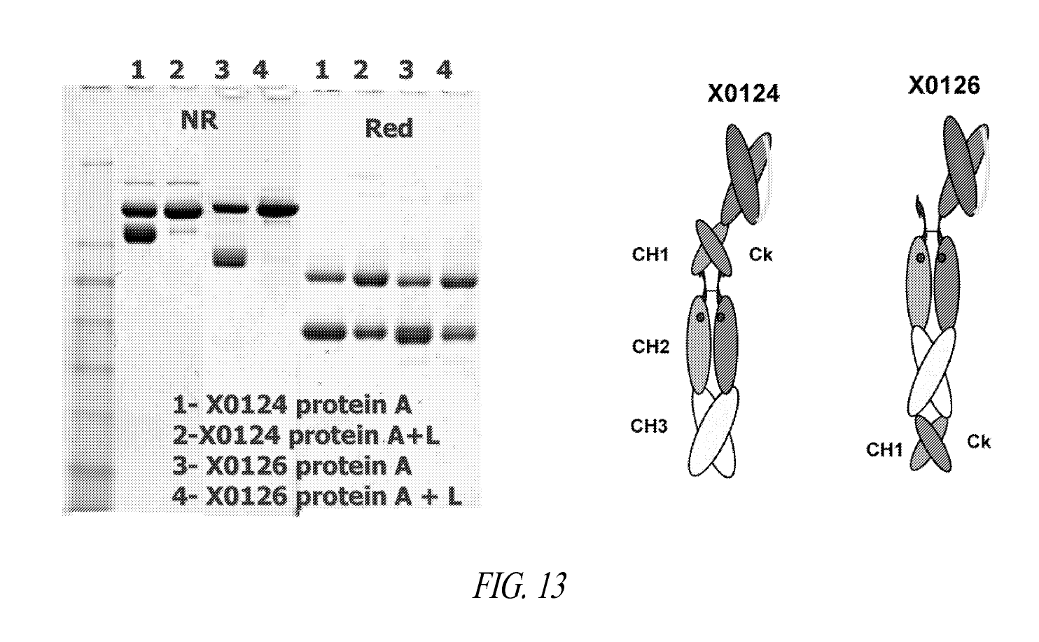

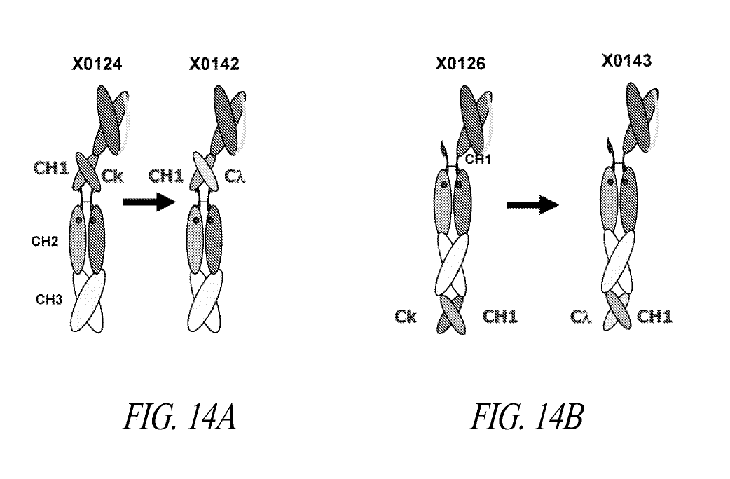

A Protein L agarose column was also used as a second step purification of X0124 and X0126. FIG. 13 show that reasonably pure heterodimer was obtained when Protein L was used as a second purification step.

3.1.7 Class 1 Interceptors with Cλ/CH1 Pair.

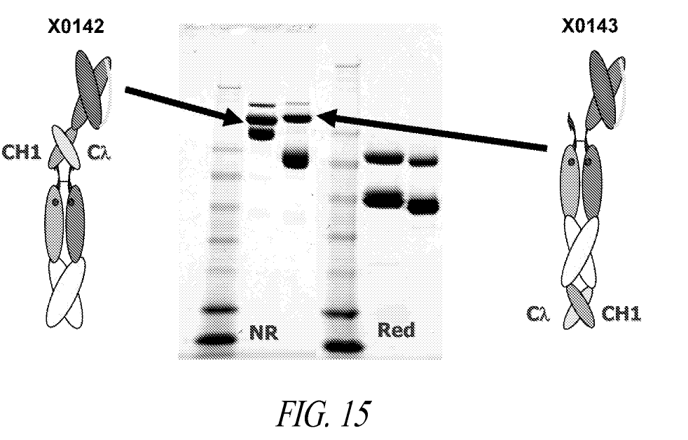

Cκ in two of the Class 1 Interceptors, X0124 and X0126, was replaced with Cλ to give X0142 and X0143, respectively (FIG. 14). In both cases, similar to the Cκ containing polypeptides, homodimer of the short chain and heterodimer were formed (FIG. 15).

3.1.8 Class 1 Interceptor with two Cκ/CH1 Pairs with Each Chain Having 1 CH1 and 1 Ck Domain.

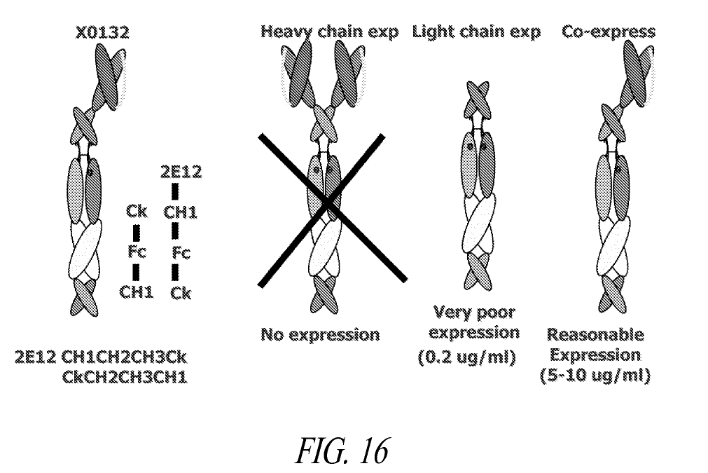

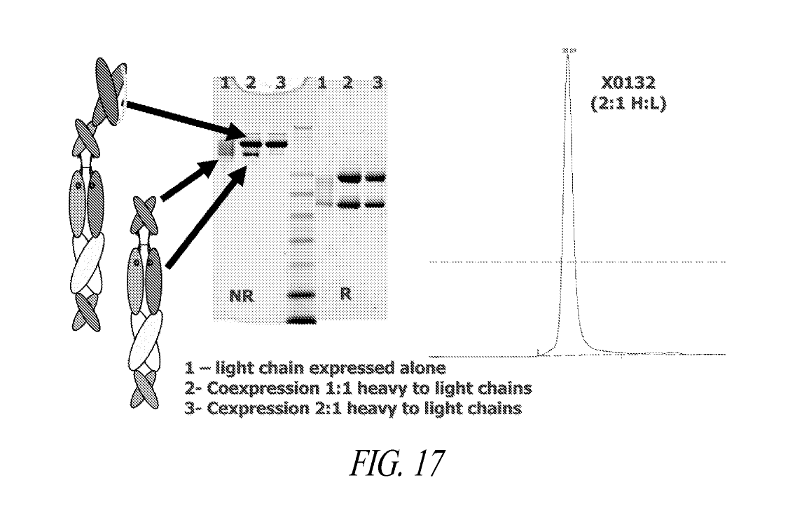

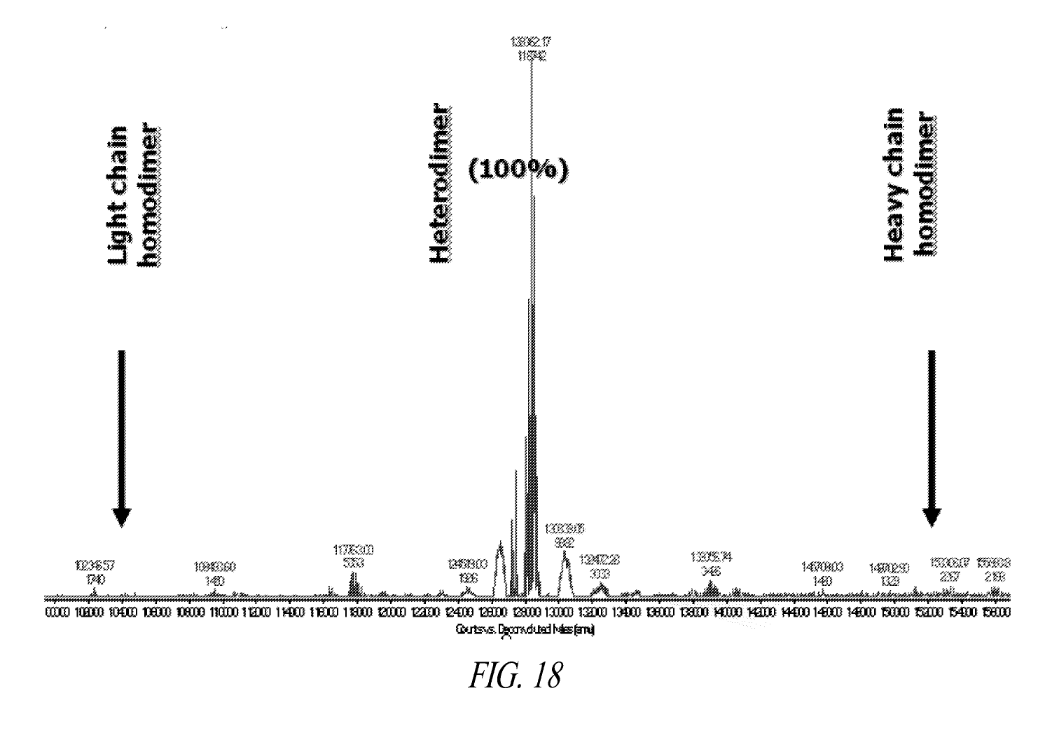

X0132 was formed by expressing X0130 and X0131. X0132 is unique in that the long chain has a binding domain and CH1 at the amino terminus and a Cκ at the carboxyl terminus of the Fc region portion, whereas the short chain has a Cκ at the amino terminus and a CH1 at the carboxyl terminus of the Fc region portion. Expression of the heavy chain alone yielded no protein, whereas expression of the light chain alone only produced very little protein. Interestingly, co-expression of the two chains resulted in reasonable production of protein (FIG. 16). If the long and short chain was co-expressed at a ratio of 2:1, very pure heterodimer was obtained (FIG. 17). Mass spectrometry analysis confirmed this by showing that neither the short chain nor the long chain homodimers were present (FIG. 18).

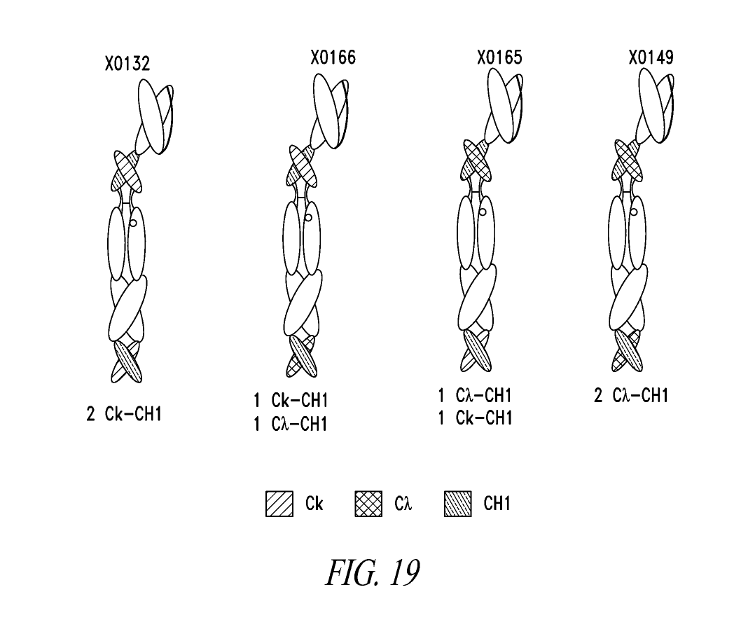

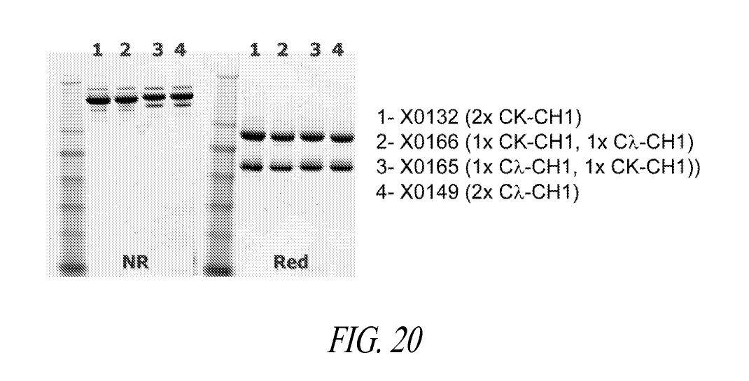

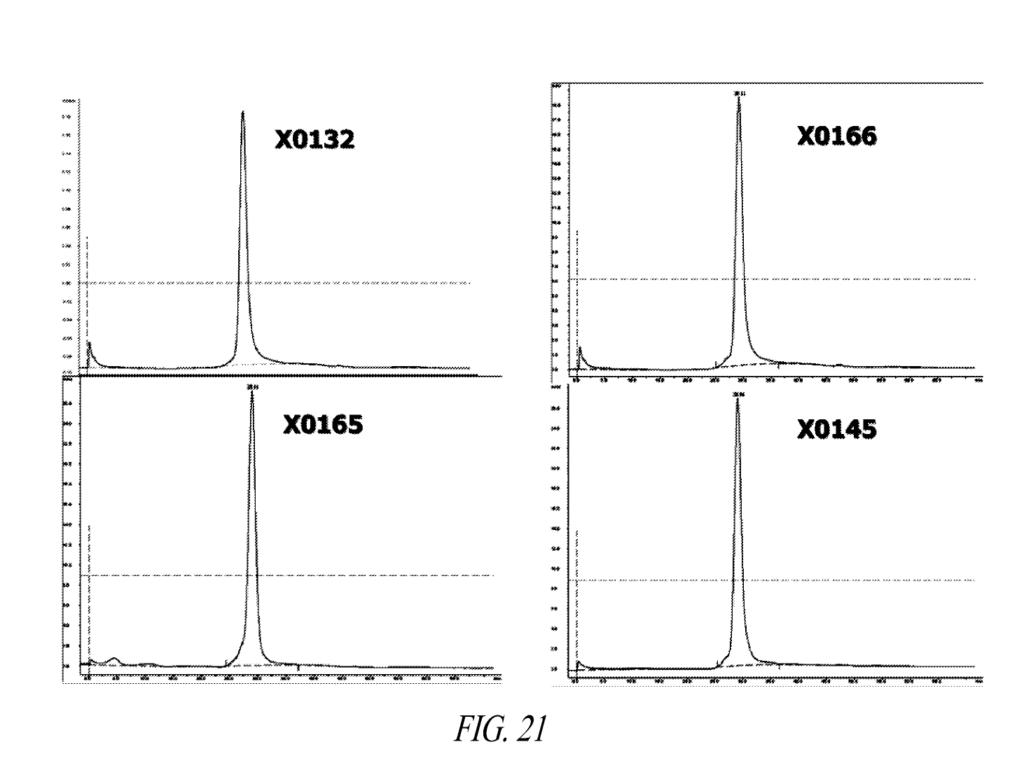

Interceptors with two CL/CH1 pairs that use a combination of Cκ and Cλ were also prepared (FIG. 19). In each case, only one CH1 and only one Cκ or Cλ is on each chain. These Interceptors X0166, X0165 and X0149 seemed to behave like X0132 in forming almost exclusively heterodimer (FIG. 20) when transfected at a ratio of 2 long chain to 1 short chain. This experiment further illustrated that Cλ may be used interchangeably in place of Cκ. The SEC analysis as shown in FIG. 21 illustrated that these Interceptors are 100% protein of interest (POI), which indicates that the formation of the heterodimer was very efficient.

### 3.1.9 FACS Binding Data for Interceptors and Other Molecular Formats

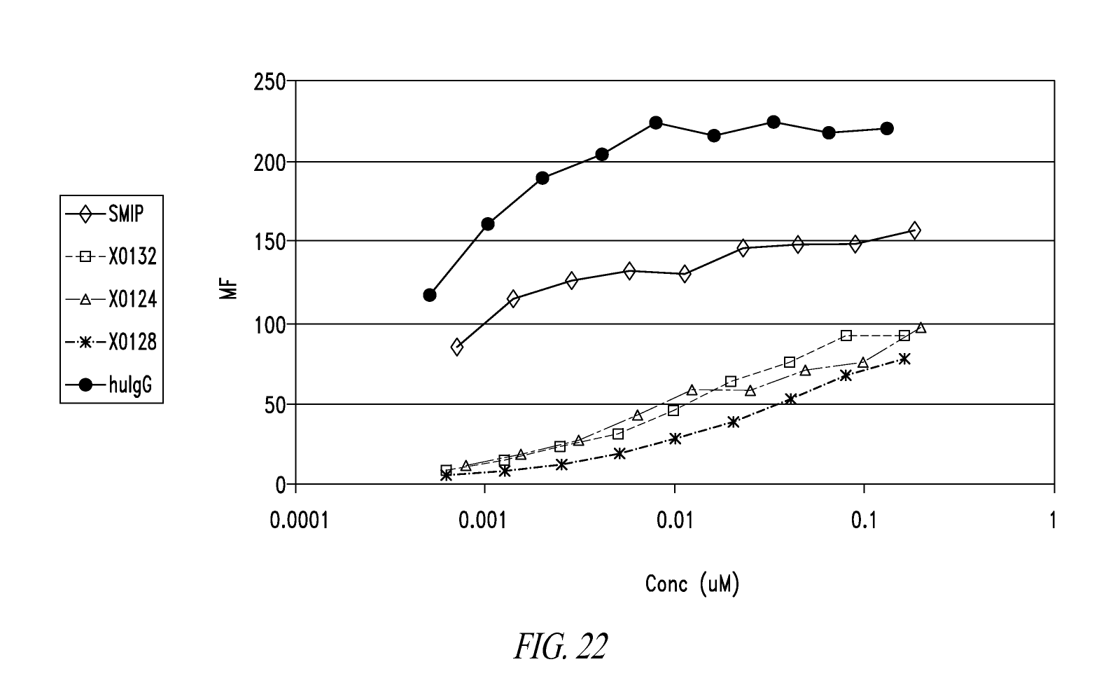

Binding of selected Interceptors, X0124, X0128 and X0132, to Jurkat cells were compared with the 2E12 binding domain expressed in other formats. As expected from the monovalent binding property of the Interceptors, they all bound less strongly than the bivalent 2E12 molecules (SMIP or huIgG) (FIG. 22)

### 3.1.10 Biological Assays on Selected Class 1 Interceptors

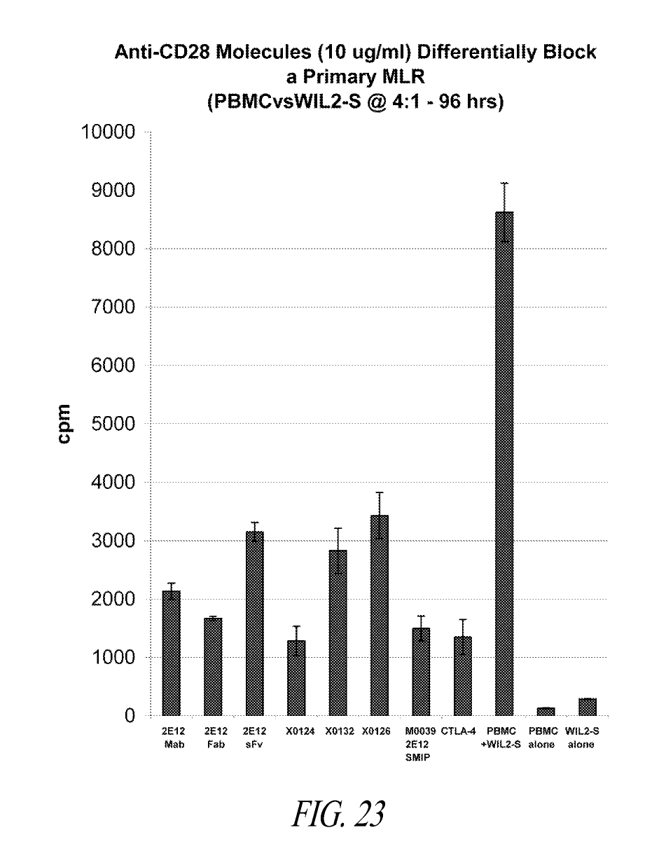

The Interceptors were tested in the following biological assays: primary MLR, secondary MLR and PMA assays. In the primary MLR assay (FIG. 23), all the Interceptors tested were able to block T cell response as well as other 2E12 molecular formats. X0124 in particular was able to block T cell response at the same level as the CTLA4-Ig molecule, which was used as a positive control.

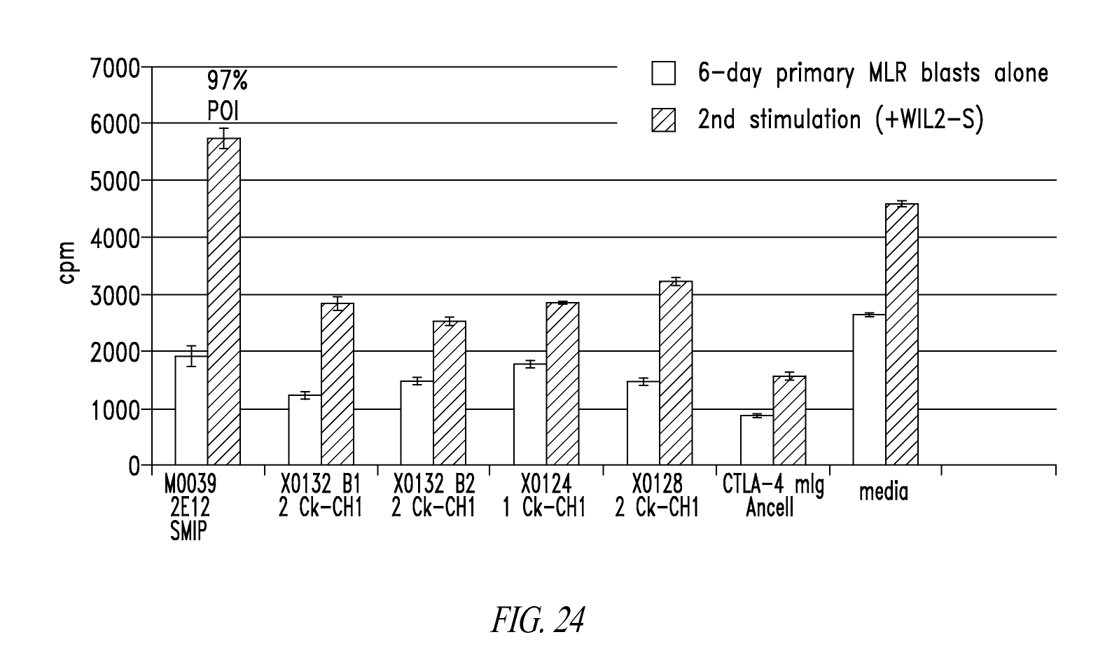

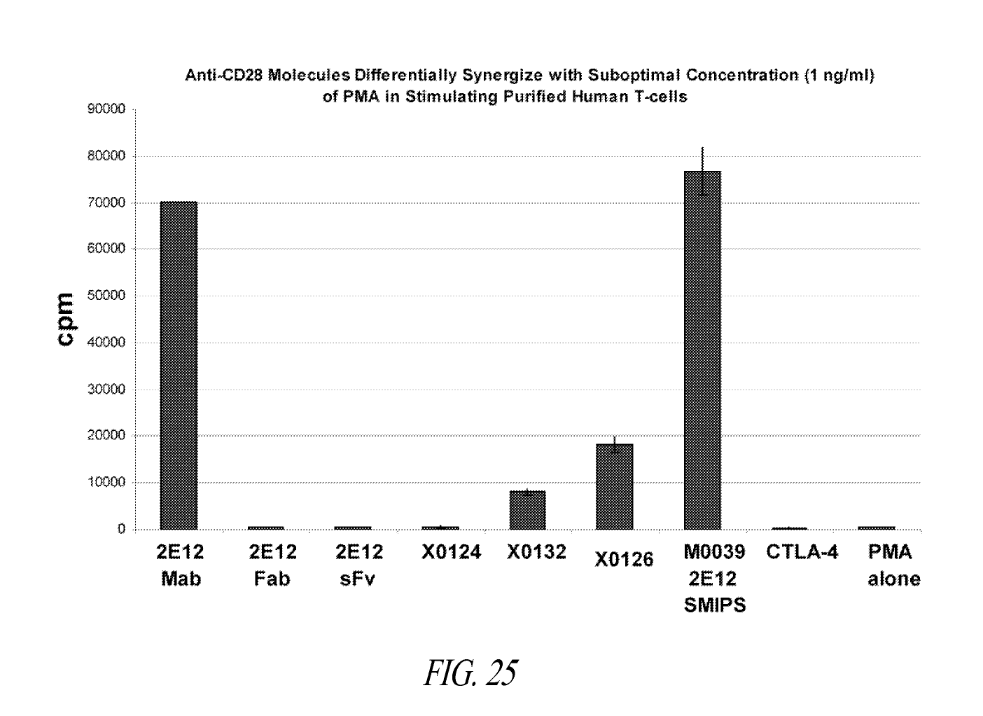

In the secondary MLR assay (FIG. 24), there was some differentiation observed—the bivalent 2E12 SMIP proteins activated the secondary T cell response whereas Interceptors blocked the response, like the control CTLA4-Ig molecule.

In the PMA assay (FIG. 25), T cells were purified from PBMC and the different 2E12 Interceptors and other 2E12 molecules were added in the presence of a suboptimal concentration of PMA (1 ng/ml). The result shows that the bivalent 2E12 molecules, such as the 2E12 SMIP protein and 2E12 huIg (also referred to as Mab or monoclonal antibody), were able to synergize with PMA in stimulating the purified T cells, whereas the Interceptors and other monovalent 2E12 molecules (like the Fab and scFv) did not.

### 3.1.11 Biophysical Characterization of Interceptors

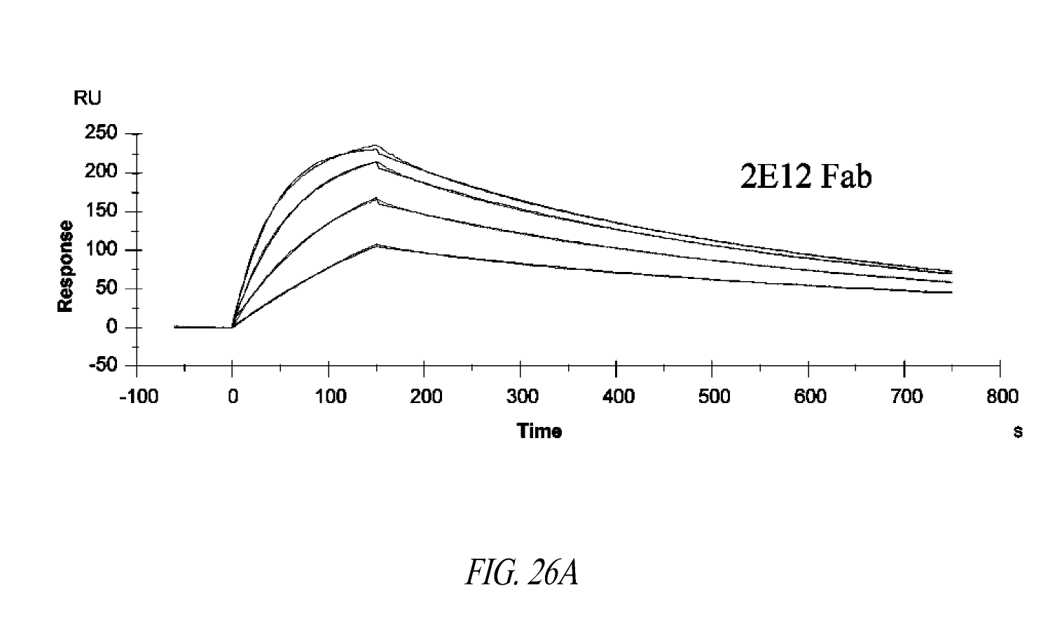

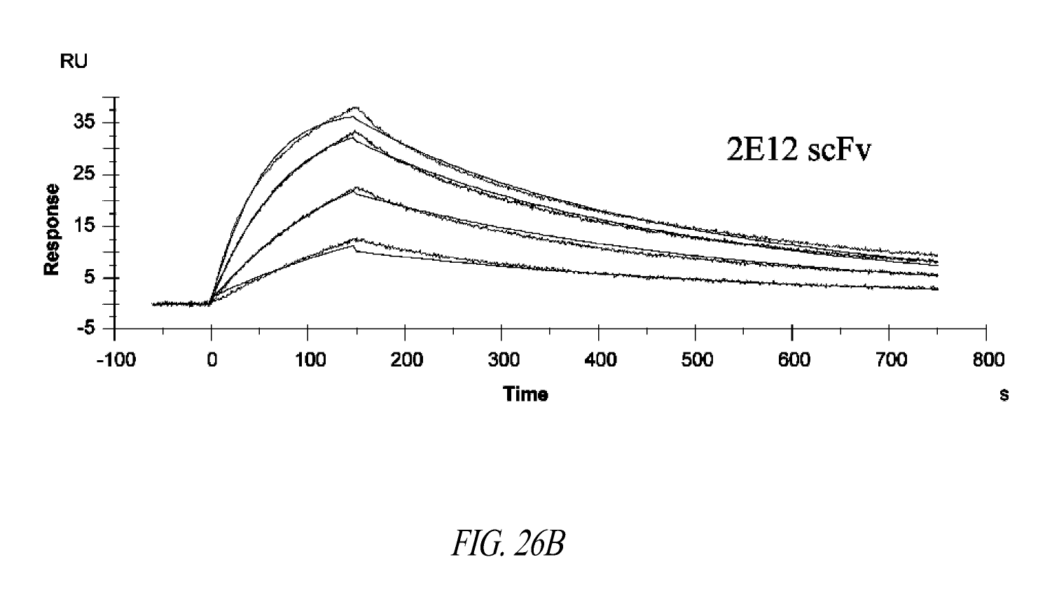

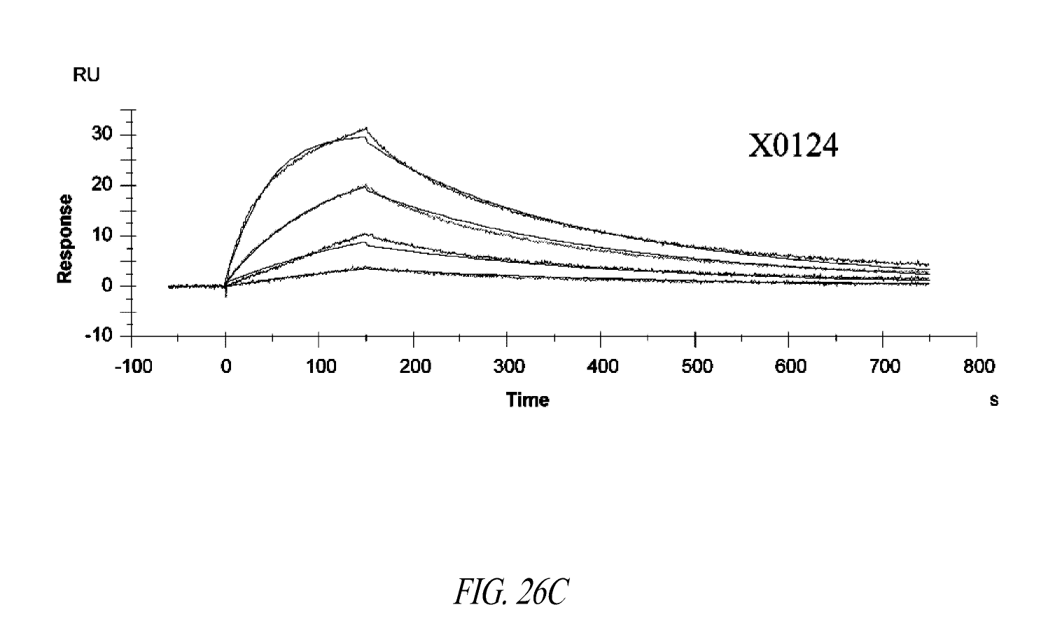

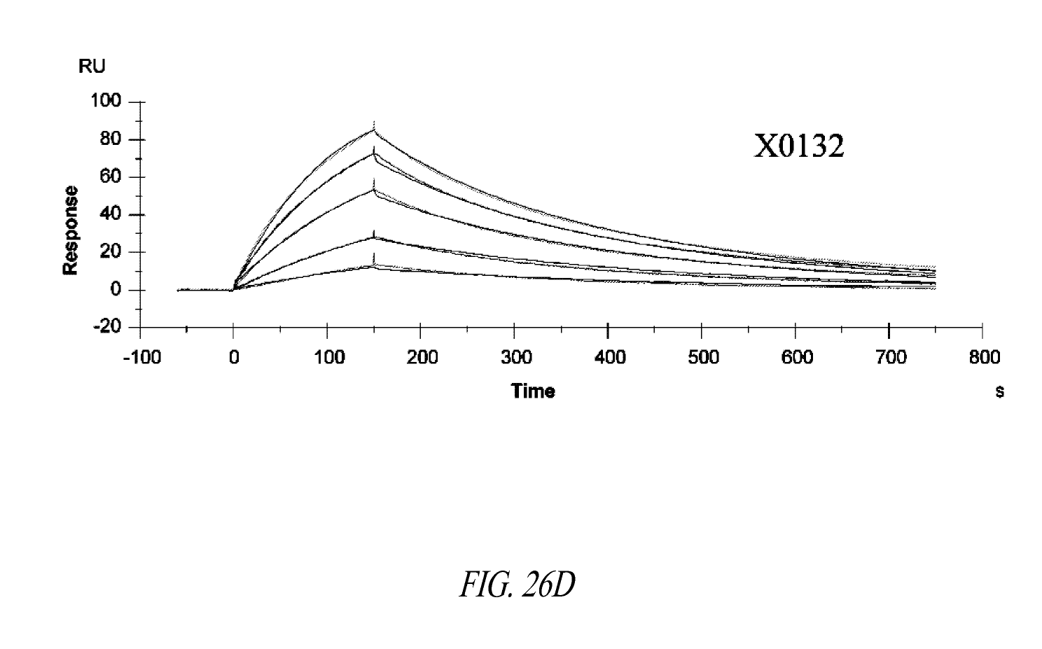

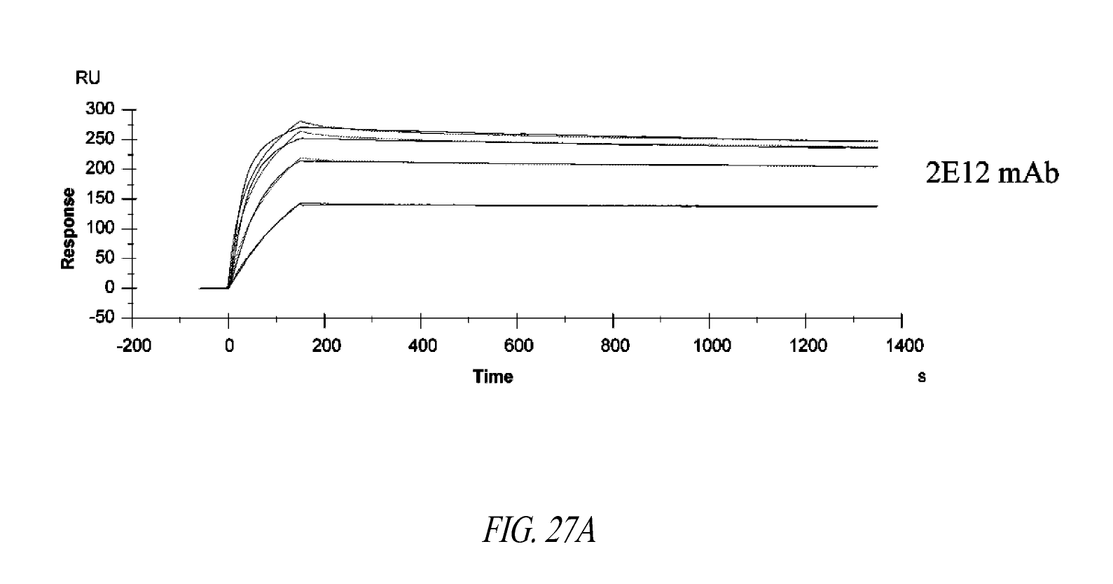

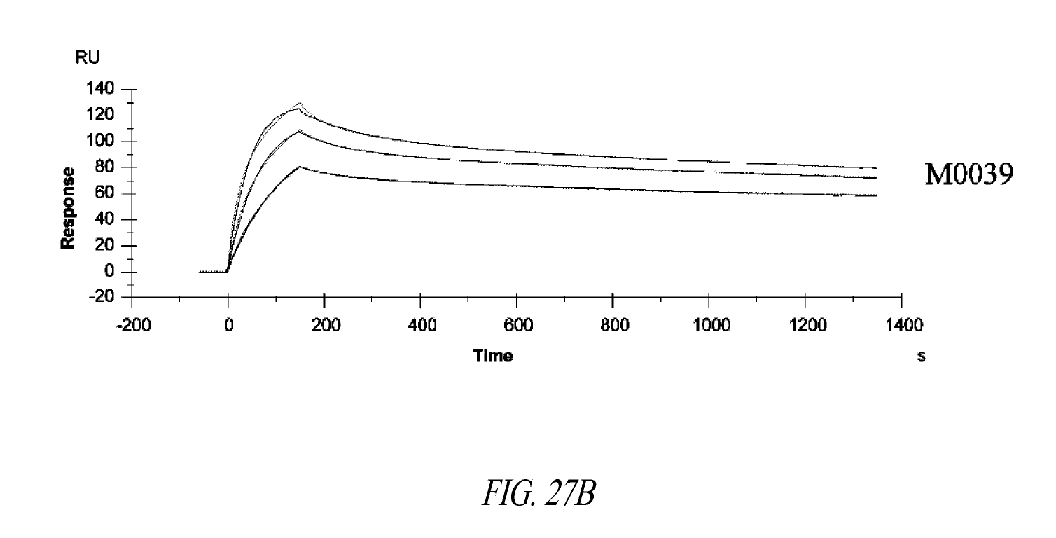

Surface plasmon resonance was used to measure binding of immobilized CD28 by 2E12 binding domain constructs. The results show that binding kinetics of the monovalent 2E12 constructs (e.g., 2E12 Fab, FIGS. 26A-26D) fit well to a 1:1 Langmuir binding model (Table 5). Binding kinetics of the bivalent 2E12 constructs (e.g., 2E12 mAb, FIGS. 27A-27B) to immobilized CD28 could not be fit to a 1:1 Langmuir binding model, but could be fit with high accuracy to a bivalent analyte binding model (Table 5). Equilibrium dissociation constants (1(D) could be calculated with high accuracy for each construct by fitting the observed response at saturation to a steady-state equilibrium model (Table 5).

TABLE 5
Binding Kinetics to immobilized CD28†

| | | | | | | Equilib- |
|---|---|---|---|---|---|---|
| | k_{a (I)} | k_{d (I)} | K_{D} | k_{a (II)} | k_{d (II)} | rium |
| Protein | (×10⁵) | (×10⁻³) | (nM) | (×10⁻³) | (×10⁻³) | K_{D} (nM) |
| 2E12 Fab | 4.6 | 2.4 | 5.24 | — | — | 28.5 |
| 2E12 mAb | 1.76 | 1.04 | 6* | 3.67 | 12.7 | 11.4 |
| 2E12 scFv | 3.48 | 3.38 | 9.69 | — | — | 39.6 |
| 2E12 SMIP | 1.7 | 4.11 | 24.2* | 0.138 | 0.3 | 19.4 |
| (M0039) | | | | | | |
| Heterodimer | 1.41 | 4.97 | 35.2 | — | — | 104 |
| 1 (X0124) | | | | | | |
| Heterodimer | 1.49 | 6.5 | 43.7 | — | — | 134 |
| 2 (X0132) | | | | | | |

†kₐ is in M⁻¹ s⁻¹, k_{d} in s⁻¹.
k_{a(I)} and kd_{(I)} are the on and off rates in a 1:1 binding model, and the first on and off rates in a bivalent analyte model.
k_{a (II)} and k_{d (II)} are the second set of on and off rates (arising from avidity) in a bivalent analyte model.
K_{D} is the kinetic dissociation constant determined from the ratio of on and off rates (kd (I)/ka (I)). Kinetic KD values shown for the bivalent analyte model (*) represent initial binding at the first site only.
Equilibrium K_{D} is the equilibrium dissociation constant.

The results show that first-site binding kinetics of all formats to CD28, are similar and within the same order of magnitude. The 2E12 binding domain bound two-fold more efficiently in the Fab format than as an scFv. This difference was reflected in the two-fold affinity difference between the mAb (containing two Fabs) and SMIP (containing two scFvs) formats. Heterodimeric polypeptides (X0124, X0132) bearing one binding domain bound monovalently to immobilized CD28, confirming that only one binding domain was displayed in the heterodimeric construct. The binding kinetics of heterodimeric polypeptides were similar to first-site binding of the SMIP format, suggesting that any steric penalty to binding of the scFv arising from attachment to a larger protein is the same between the two formats.

### 3.2 Class 2 Interceptors

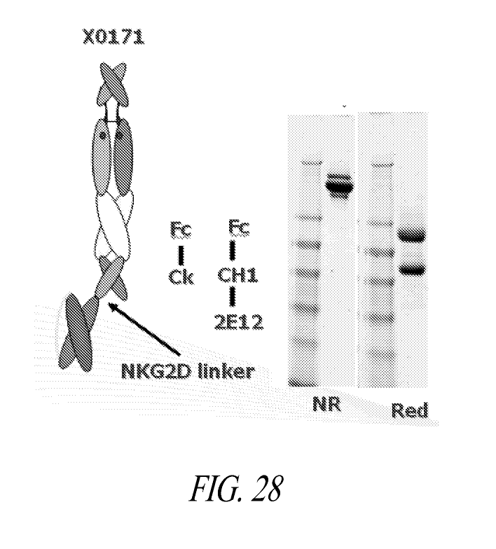

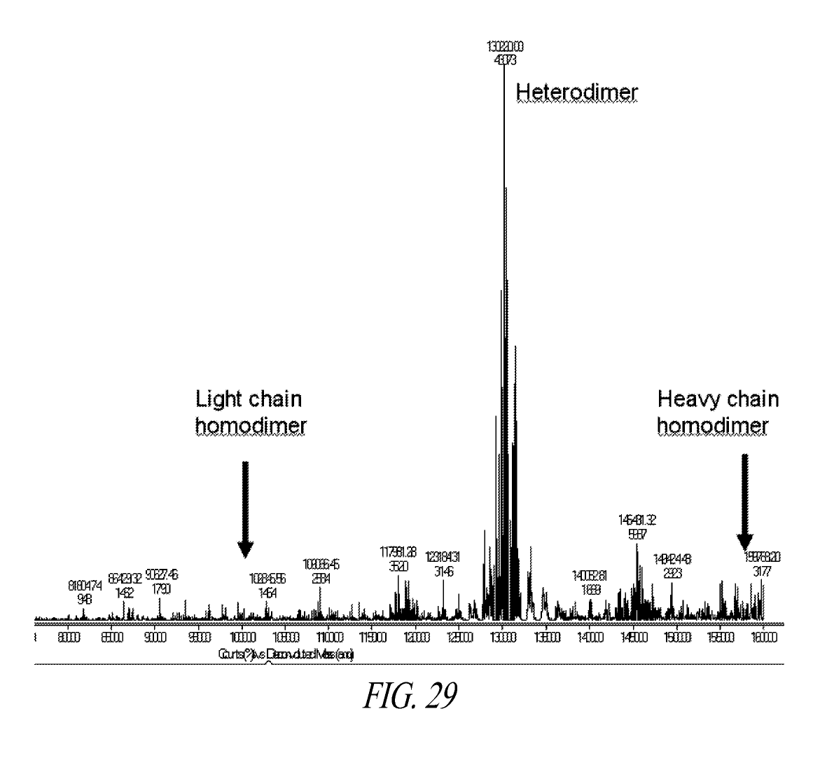

A class 2 Interceptor (X0171) where the binding domain was placed on the backend of the molecule was made. This molecule has 2 CH1-Ck pairs and the 2E12 scFv is located at the C-terminus of CH1 via an NKG2D linker. FIG. 28 shows the SDS-PAGE results of X0171 and FIG. 29 illustrates the mass spectrum of X0171, showing that the polypeptide heterodimer was the predominant species.

### 3.3 Cation Exchange Chromatographic Analysis of Interceptors

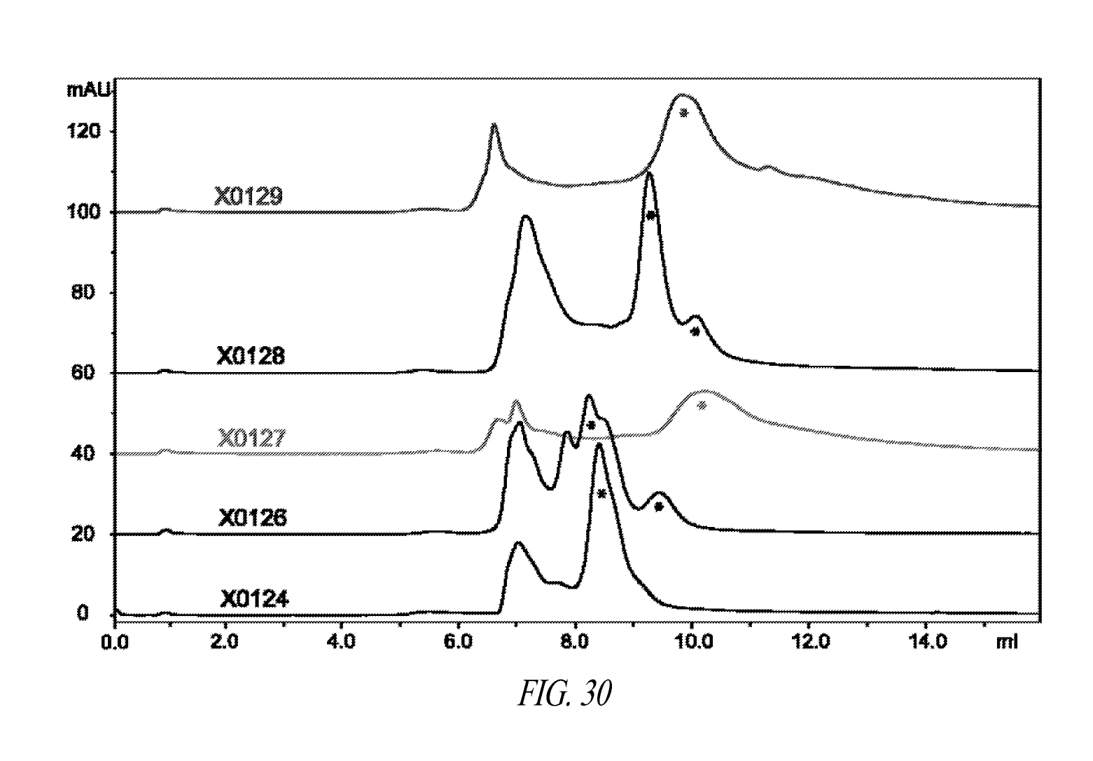

Analytical separation of homodimeric and heterodimeric molecules was attained by standard cation exchange chromatography (FIG. 30), with homodimeric molecules eluting at a lower salt concentration than heterodimeric molecules. As is typical with ion exchange chromatography, different charge states of the same molecule eluted at different salt concentrations, thus multiple peaks were occasionally observed for either homodimeric or heterodimeric molecules.

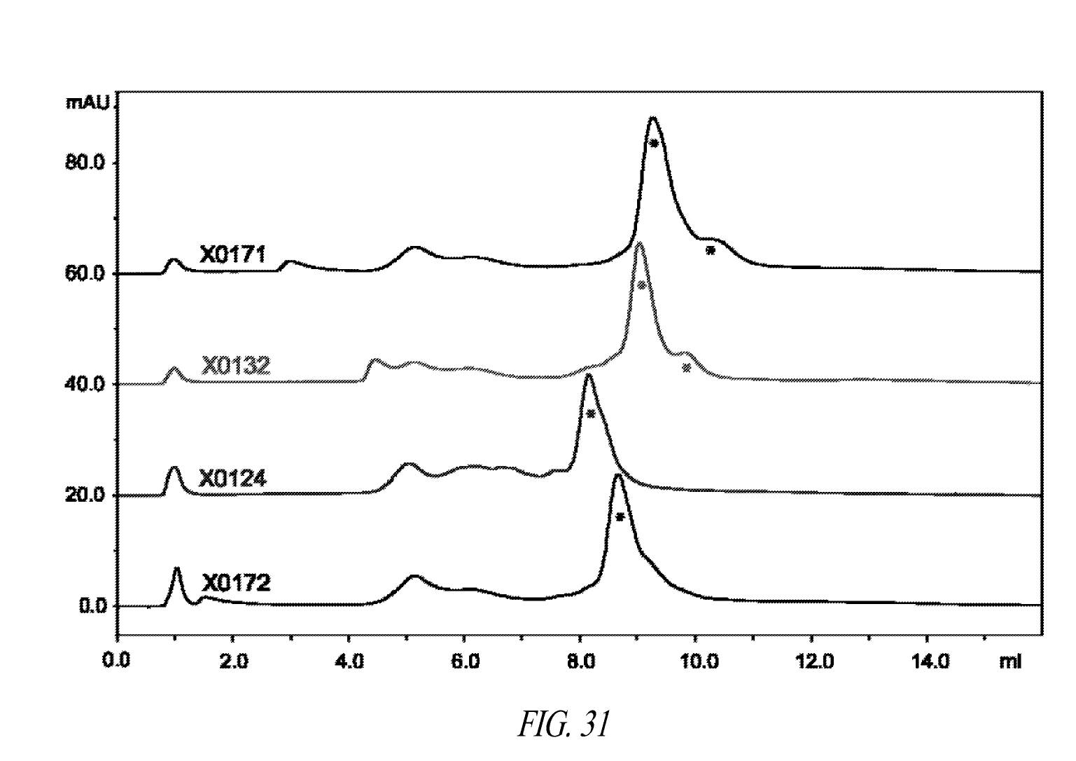

This assay confirmed the efficacy of reversing the orientation of the CH1/Cκ or CH1/Cλ pair on the long single chain fusion polypeptide relative to the short single chain fusion polypeptide (X0132, X0171, X0172, FIG. 31) and that a secondary Protein L affinity purification step (X0124, FIG. 31) was effective at highly enriching the heterodimer population.

### 3.4 Mutated Interceptors

As shown herein, pure or substantially pure (over 90% or 95% pure) heterodimers were obtained with Interceptors having one CH1-Cκ (or Cλ) pair using a secondary purification via ion exchange or protein L affinity chromatography. Alternatively, substantially pure (over 90% or 95% pure) heterodimers were obtained with Interceptors having two CH1-Cκ (Cλ) pairs and using excess long chain DNA during the transfection step, which did not require a secondary purification. However, the Interceptors with two CH1-Cκ (Cλ) pairs have higher molecular weights than those with one CH1-Cκ (Cλ) pair by about 23 kDa.

Alternative embodiments were considered that have one, two or all of the following characteristics: first, it uses only one CH1-Cκ (Cλ) pair to reduce the size of the molecule to the minimum; second, it forms pure or substantially pure (over 90% or 95% pure) heterodimer to avoid secondary purification; and lastly, this heterodimerization is compatible with null mutations in the Fc domains if effector function is not needed.

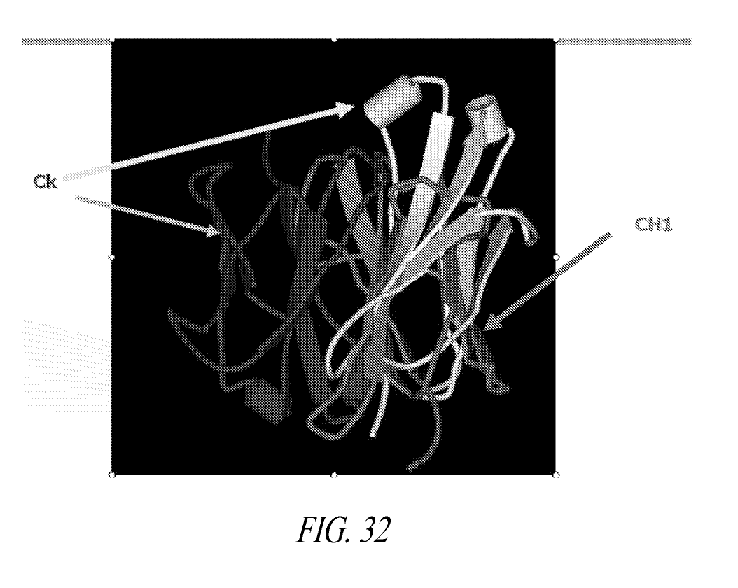

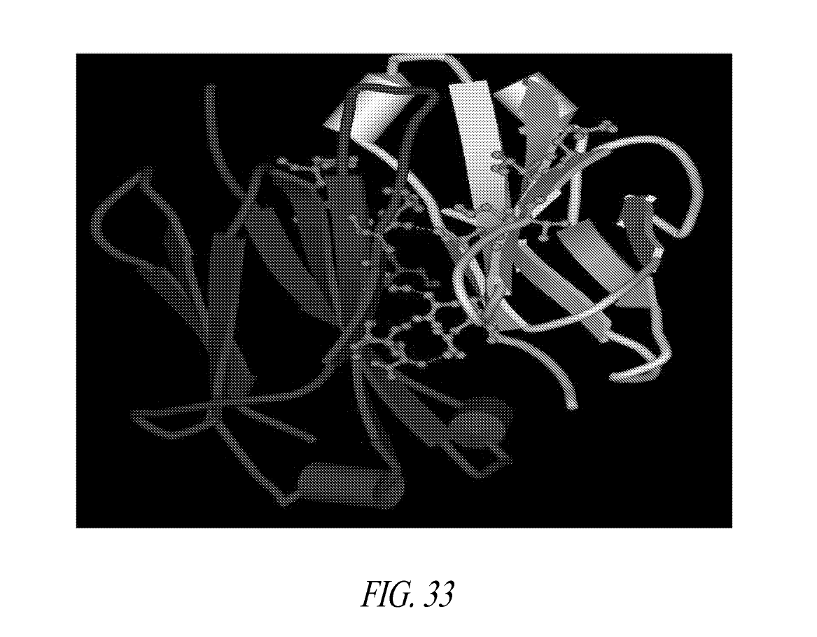

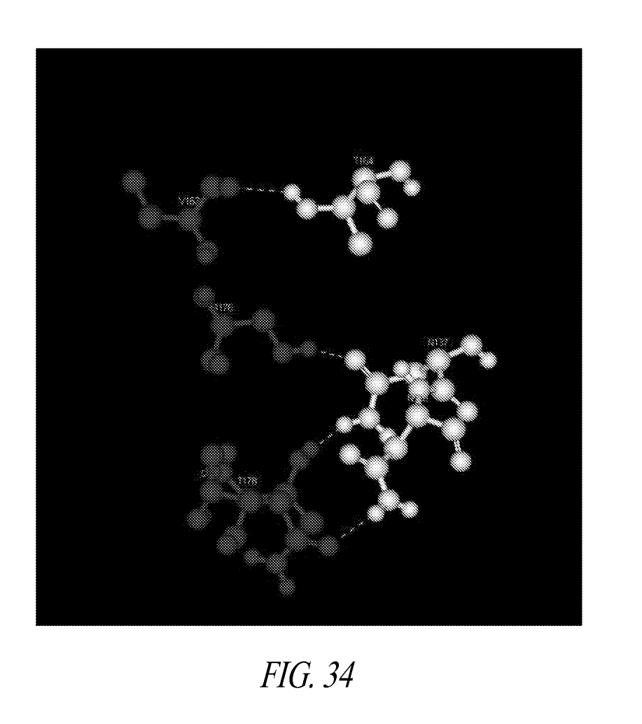

The crystal structure of Ck-Ck versus Ck-CH1 (FIG. 32) shows that the homodimer interface formed by Ck and Ck is different from the heterodimer interface of Ck-CH1 and that the inter-hydrogen network at the Ck-Ck interface (FIGS. 33 and 34) is not present in the Ck-CH1 interface. The differences were used to introduce rational mutations that could destabilize the Ck-Ck interface without affecting the Ck-CH1 interface.

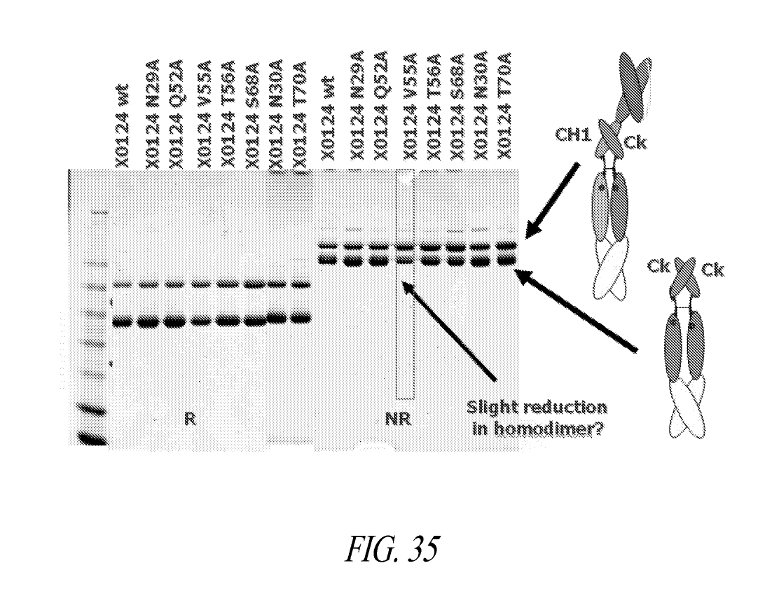

Seven residues that were identified as forming the inter-hydrogen bonding network at the Ck-Ck interface (N29, N30, Q52, V55, T56, S68 and T70) were mutated to alanine residues on the X0113 chain. X0112 and X0113 were co-expressed and proteins examined on SDS-PAGE to determine relative ratio of heterodimer and short chain homodimer. FIG. 35 shows that the expression level for all the mutants seemed to be comparable to the wild type X0124 and that the V55A mutation seemed to slightly favor the heterodimer species.

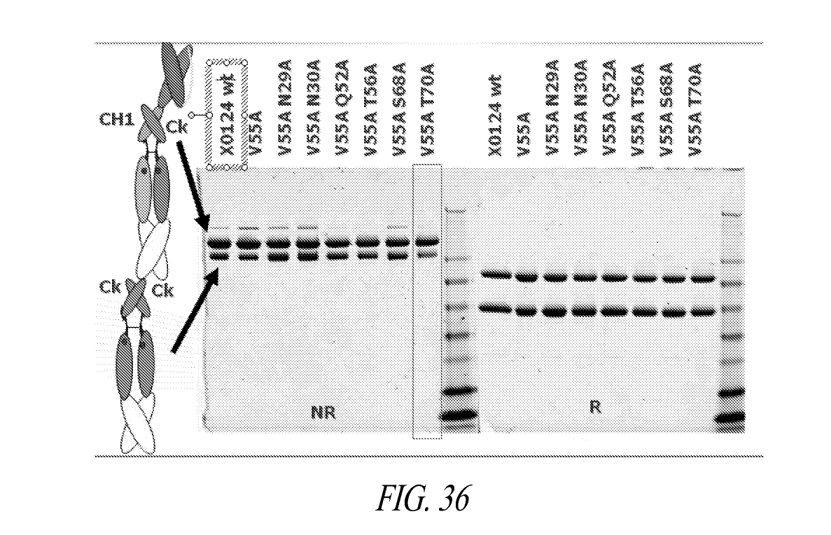

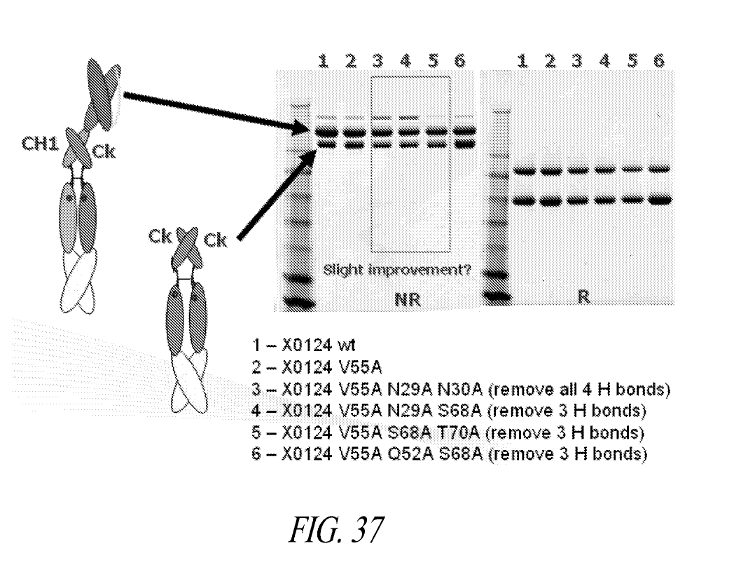

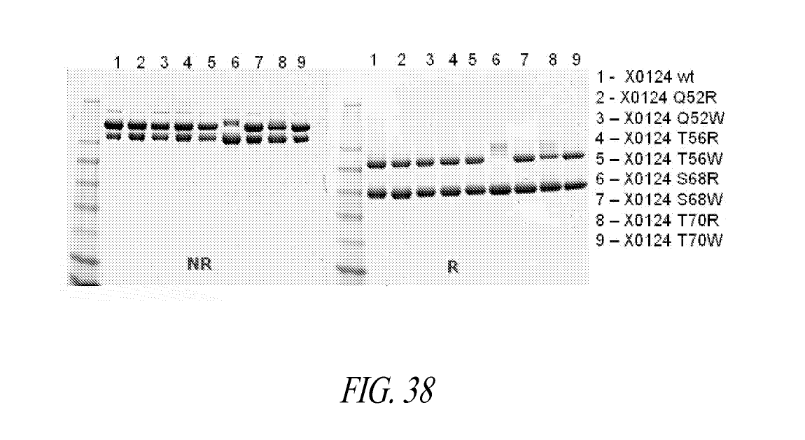

Double alanine mutations were next introduced on X0113 with V55A being one of the fixed mutations. FIG. 36 shows that the X0124 V55A T70A mutants seemed to have improved heterodimerization. Triple alanine mutations were also introduced which would remove 3 or 4 of the hydrogen bonds in the interface. As shown in FIG. 37, there was slight improvement in the heterodimerization for three of four triple mutants tested.

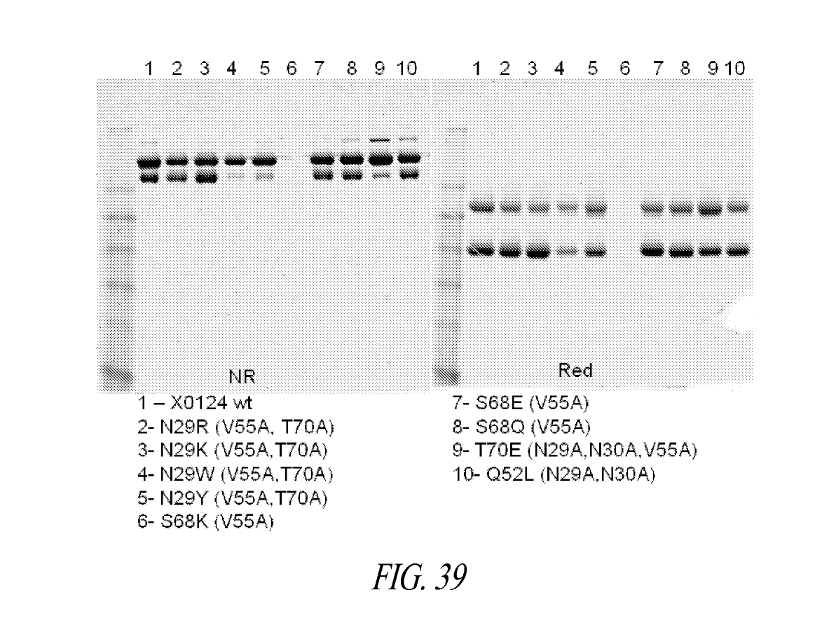

Bulky side chain amino acid mutations were also introduced individually at four of the interface residues: Q52, T56, S68 and T70, with arginine (R) or trytophan (W) replacing the wild type residues at these positions. FIG. 38 shows that a couple of these single point mutants might help heterodimerization, but some may unexpectedly promote homodimerization.

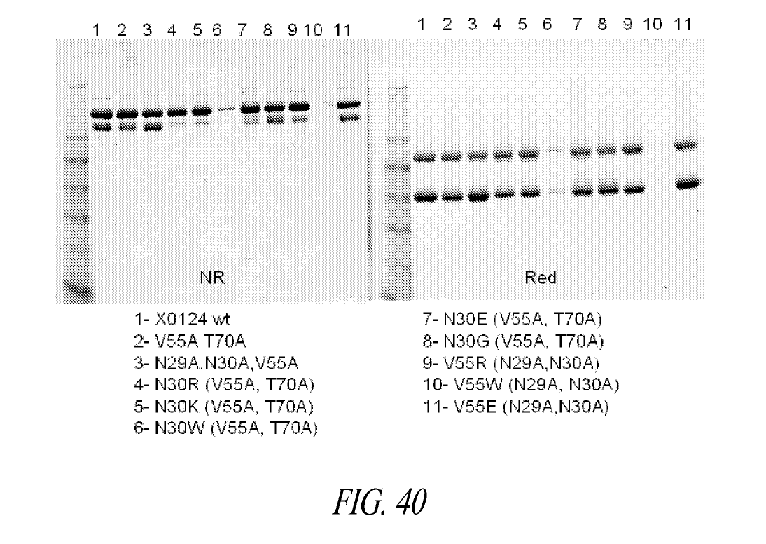

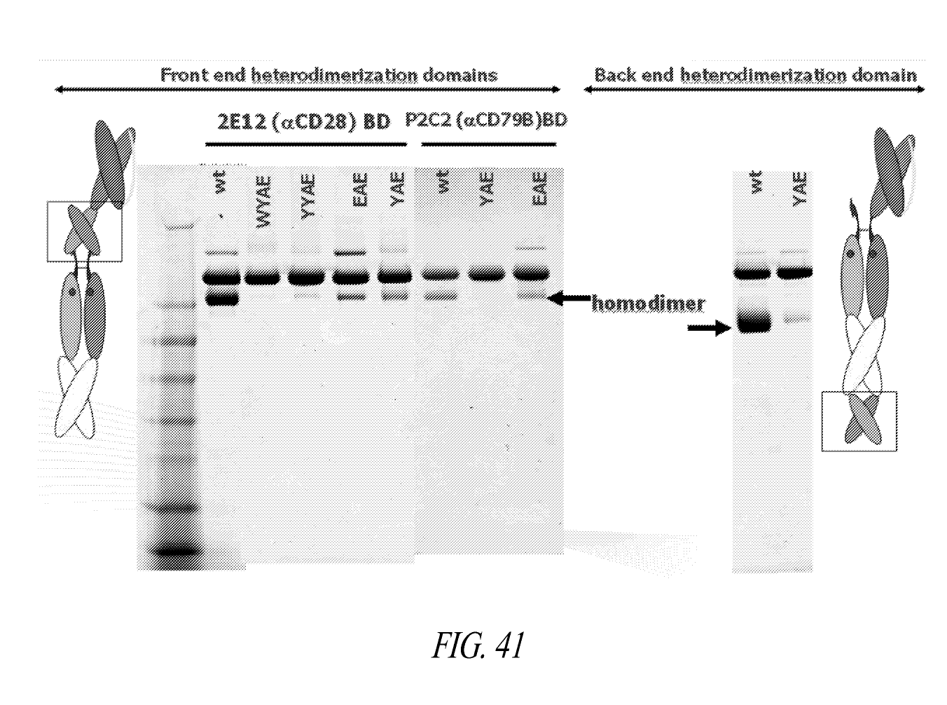

A wider range of bulky side chain amino acid mutations were also introduced into these positions (N29, N30, Q52, V55, S68, and T70) and combined that with alanine mutations elsewhere. FIGS. 39 and 40 show that seven of the mutants appeared to be beneficial in destabilizing homodimer formation and resulting in a significantly higher percentage of heterodimer formation (greater than 90% heterodimer in some cases). These mutations are as follows: (1) N29W,V55A,T70A (SEQ ID NO:91), (2) N29Y,V55A,T70A (SEQ ID NO:92), (3) T70E, N29A,N30A,V55A (SEQ ID NO:193), (4) N30R,V55A,T70A (SEQ ID NO:98), (5) N30K,V55A,T70A (SEQ ID NO:99), (6) N30E, V55A,T70A (SEQ ID NO:101) and (7) V55R,N29A,N30A (SEQ ID NO:103). These results show that beneficial mutations can include bulky amino acids at positions 29, 30, 55 or 70 in combination with other mutations that disrupt CL homodimerization.

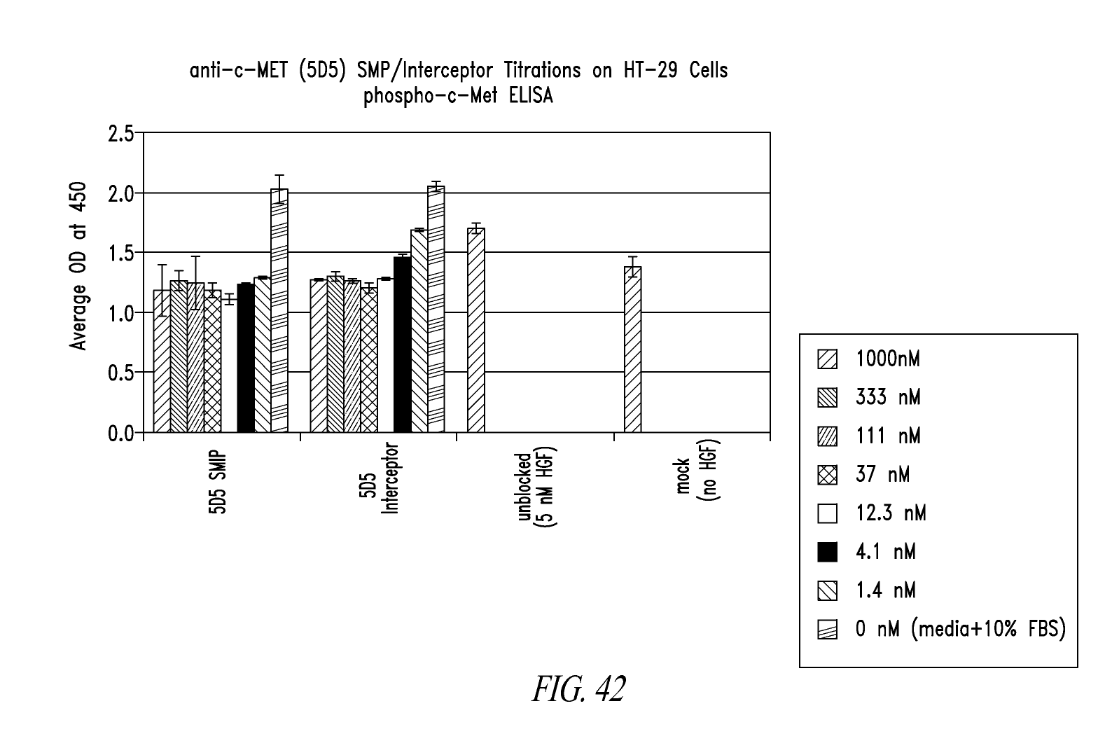

Additional bulky side chain amino acid mutations in combination with alanine mutations at positions 29, 30, 55 or 70 were further investigated (see, Table 6). The SDS-PAGE (non-reducing condition) results (FIG. 41, left panel) shows that for 2E12 binding domain, 4 mutations [WYAE (N29W N30Y V55A T70E) and YYAE (N29Y N30Y V55A T70E)] on the Ck domain can result in near 100% heterodimerization, whereas triple mutations [EAE (N30E V55A T70E) and YAE (N30Y V55A T70E))] result in over 90% heterodimerization. For the P2C2 binding domain (FIG. 41, left panel), it appeared that a triple mutation (particularly YAE) is sufficient to achieve close to 100% heterodimerization. Constructs containing the mutated Ck heterodimerization domain at the C-terminus of the CH2 and CH3 domains achieved over 90% heterodimerization (FIG. 41, right panel).

TABLE 6
Mutated Interceptors

| Interceptors | Name | Chain 1 | Chain 2 |
|---|---|---|---|
| Front end 2E12 wt | X0124 | X0112 (SEQ ID NO: 22) | X0113 (SEQ ID NO: 24) |
| WYAE (N29W N30Y V55A | X0232 | X0112 (SEQ ID NO: 22) | X0229 (SEQ ID NO: 127) |
| T70E) | | | |
| YYAE (N29Y N30Y V55A | X0233 | X0112 (SEQ ID NO: 22) | X0231 (SEQ ID NO: 129) |
| T70E) | | | |
| EAE (N30E V55A T70E) | X0211 | X0112 (SEQ ID NO: 22) | X0193 (SEQ ID NO: 131) |
| YAE (N30Y V55A T70E) | X0224 | X0112 (SEQ ID NO: 22) | X0220 (SEQ ID NO: 133) |
| Front End P2C2 wt | X0235 | X0234 (SEQ ID NO: 135) | X0113 (SEQ ID NO: 24) |
| YAE (N30Y V55A T70E) | X0236 | X0234 (SEQ ID NO: 135) | X0220 (SEQ ID NO: 133) |
| EAE (N30E V55A T70E) | X0237 | X0234 (SEQ ID NO: 135) | X0193 (SEQ ID NO: 131) |
| Back end 2E12 wt | X0126 | X0115 (SEQ ID NO: 26) | X0114 (SEQ ID NO: 28) |
| Back end YAE (N30Y | X0238 | X0115 (SEQ ID NO: 26) | X0225 (SEQ ID NO: 137) |
| V55A T70E) | | | |

### Example 2

### C-Met Specific Interceptor Blocks HGF-Induced Phosphorylation of C-Met

The 5D5 binding domain inhibits the activation of the human c-Met receptor tyrosine kinase by its ligand, known as hepatocyte growth factor or scatter factor (HGF) (Jin et al. (2008) Cancer Research 68:4360-4368). The 5D5 hybridoma was converted to the corresponding SMIP and Interceptor scaffolds, and tested for the ability to inhibit HGF-induced receptor activation. The 5D5 Interceptor was formed by coexpressing a first single chain polypeptide that comprises from its amino-terminus to carboxy-terminus, 5D5scFv, human IgG1 CH1, human IgG1 CH2, human IgG1 CH3, and human Cκ as set forth in SEQ ID NO:139 and a second single chain polypeptide, X0131, that comprises from its amino-terminus to carboxy-terminus, human IgG1 Cκ, human IgG1 CH2, human IgG1 CH3, and human CH1 as set forth in SEQ ID NO:48.

To measure the ability of our molecules to block HGF-induced phosphorylation of c-MET, approximately 30,000 HT-29 cells were plated per well in a 96-well plate in RPMI 1640 +10% Fetal Bovine Serum (FBS). The following day, media was aspirated, and cells were treated with 50 μl of blocking solution diluted in RPMI 1640 without FBS for 1 hour at 37° C. Aspirated blocking treatments were aspirated, and 50 μl of mock treatment (RPMI 1640, 1 mM activated sodium orthovanadate) or rhHGF treatment (RPMI 1640, 1 mM activated sodium orthovanadate, 5 nM rhHGF) was added. The resulting mixture was incubated for 10 min. at room temperature. Media was aspirated again and cells were lysed in ice-cold 1X Sample Diluent Concentrate 2 supplemented with 1 mM activated sodium orthovanadate, 1X Halt™ Protease Inhibitors and 1X Halt™ Phosphatase Inhibitors. Lysates were frozen at −20° C. before analysis on the DuoSet IC Human Phospho-HGF R/c-MET ELISA, according to manufacturer's instructions. The 1 mM activated sodium orthovanadate included in the mock and rhHGF treatments prevents dephosphorylation of c-MET in the absence of rhHGF, leading to higher levels of background phosphorylation on the c-MET receptor than would be observed if a lower concentration of activated sodium orthovanadate had been used in the treatments.

rhHGF, Sample Diluent Concentrate 2, and the DuoSet IC Human Phospho-HGF R/c-MET ELISA were purchased from R&D Systems (Minneapolis, Minn.). Halt™ Protease and Phosphatase Inhibitor Cocktails were purchased from Thermo Fisher Scientific (Rockford, Ill.). The HT-29 cell line was obtained from the American Type Culture Collection (ATCC, Manassas, Va.).

Both the 5D5 SMIP and the 5D5 Interceptor showed dose-dependent inhibition of c-Met phosphorylation in response to HGF treatment, with the (bivalent) 5D5 SMIP showing efficient suppression of c-Met phosphorylation at a concentration of 1.4 nM, and the (monovalent) 5D5 Interceptor showing efficient suppression of phosphorylation at a concentration of 12 nM (FIG. 42).

### Example 3

### Polypeptide Heterodimers having Mutated Ck Domains

Several additional polypeptide heterodimers having mutated Ck domains were made.

Polypeptide heterodimer X0306 comprises single chain polypeptides X0303 and X0294. Single chain polypeptide X0303 comprises from its amino to carboxyl terminus: humanized Cris-7 (anti-CD3) (VH3-VL1) scFv, human IgG1 SCC-P hinge, mutated human IgG1 CH2 having alanine at positions 234, 235, 237, 318, 320, and 322, human IgG1 CH3, and human CH1. The nucleotide and amino acid sequences of X0303 are set forth in SEQ ID NOS: 764 and 769, respectively. Single chain X0294 comprises from its amino to carboxyl terminus: human IgG1 SCC-P hinge, mutated human IgG1 CH2 having alanine at positions 234, 235, 237, 318, 320, and 322, human IgG1 CH3, and mutated human Ck that does not contain its carboxyl-terminal cysteine and contains N30D V55A T70E substitutions (DAE). The nucleotide and amino acid sequences of X0294 are set forth in SEQ ID NOS:760 and 765, respectively.

Polypeptide heterodimer X0308 comprises single chain polypeptides X0303 and X0296. Single chain polypeptide X0296 comprises its amino to carboxyl terminus: human IgG1 SCC-P hinge, mutated human IgG1 CH2 having alanine at positions 234, 235, 237, 318, 320, and 322, human IgG1 CH3, and mutated human Ck that does not contain its carboxyl-terminal cysteine and contains N30M V55A T70E substitutions (MAE). The nucleotide and amino acid sequences of X0296 are set forth in SEQ ID NOS:761 and 766, respectively.

Polypeptide heterodimer X0309 comprises single chain polypeptides X0303 and X0297. Single chain polypeptide X0297 comprises its amino to carboxyl terminus: human IgG1 SCC-P hinge, mutated human IgG1 CH2 having alanine at positions 234, 235, 237, 318, 320, and 322, human IgG1 CH3, and mutated human Ck that does not contain its carboxyl-terminal cysteine and contains N30S V55A T70E substitutions (SAE). The nucleotide and amino acid sequences of X0297 are set forth in SEQ ID NOS:762 and 767, respectively.

Polypeptide heterodimer X0308 comprises single chain polypeptides X0303 and X0298. Single chain polypeptide X0298 comprises its amino to carboxyl terminus: human IgG1 SCC-P hinge, mutated human IgG1 CH2 having alanine at positions 234, 235, 237, 318, 320, and 322, human IgG1 CH3, and mutated human Ck that does not contain its carboxyl-terminal cysteine and contains N30F V55A T70E substitutions (FAE). The nucleotide and amino acid sequences of X0298 are set forth in SEQ ID NOS:763 and 768, respectively.

Polypeptide heterodimers X0306, X0308, X0309 and X0310 were expressed according to Example 1. The following expression levels were obtained: 26.8 μg protein/mL of culture for heterodimer X0306, 13.3 μg protein/mL of culture for heterodimer X0308, 18.9 μg protein/mL of culture for heterodimer X0309, and 5.9 μg protein/mL of culture for heterodimer X0310.

### Example 4

### Polypeptide Heterodimers Having Mutated CH1 and Ck Domains for Forming Salt Bridges

Several additional polypeptide heterodimers having mutated CH1 and Ck domains for forming salt bridges were made.

Polypeptide heterodimer X0311 comprises single chain polypeptides X0299 and X0302. Single chain polypeptide X0299 comprises from its amino to carboxyl terminus: human IgG1 SCC-P hinge, mutated human IgG1 CH2 having alanine at positions 234, 235, 237, 318, 320, and 322, human IgG1 CH3, and mutated human Ck that does not contain its carboxyl-terminal cysteine and contains an L29E substitution. The nucleotide and amino acid sequences of X0299 are set forth in SEQ ID NOS:774 and 778, respectively. Single chain polypeptide X0302 comprises from its amino to carboxyl terminus: humanized Cris-7 (anti-CD3) (VH3-VL1) scFv, human IgG1 SCC-P hinge, mutated human IgG1 CH2 having alanine at positions 234, 235, 237, 318, 320, and 322, human IgG1 CH3, and mutated human CH1 having a V68K substitution. The nucleotide and amino acid sequences of X0302 are set forth in SEQ ID NOS:777 and 781, respectively.

Polypeptide heterodimer X0312 comprises single chain polypeptides X0300 and X0301. Single chain polypeptide X0300 comprises from its amino to carboxyl terminus: human IgG1 SCC-P hinge, mutated human IgG1 CH2 having alanine at positions 234, 235, 237, 318, 320, and 322, human IgG1 CH3, and mutated human Ck that does not contain its carboxyl-terminal cysteine and contains an L29K substitution. The nucleotide and amino acid sequences of X0300 are set forth in SEQ ID NOS:775 and 779, respectively. Single chain polypeptide X0301 comprises from its amino to carboxyl terminus: humanized Cris-7 (anti-CD3) (VH3-VL1) scFv, human IgG1 SCC-P hinge, mutated human IgG1 CH2 having alanine at positions 234, 235, 237, 318, 320, and 322, human IgG1 CH3, and mutated human CH1 having a V68E substitution. The nucleotide and amino acid sequences of X0301 are set forth in SEQ ID NOS:776 and 780, respectively.

Polypeptide heterodimers X0311 and X0312 were expressed according to Example 1. The following expression levels were obtained: 32 μg protein/mL of culture for heterodimer X0311 and 38 μg protein/mL of culture for heterodimer X0312.

The various embodiments described above can be combined to provide further embodiments. All of the patents, patent application publications, patent applications, and non-patent publications referred to in this specification and/or listed in the Application Data Sheet, are incorporated herein by reference, in their entirety. Aspects of the embodiments can be modified to employ concepts of the various patents, applications and publications to provide yet further embodiments.

These and other changes can be made to the embodiments in light of the above-detailed description. In general, the terms used in the claims should not be construed to limit the claims to the specific embodiments disclosed in the specification or recited in the claims, but should be construed to include all possible embodiments and the full scope of equivalents to which such claims are entitled. Accordingly, the claims are not limited by this disclosure.

1. A polypeptide heterodimer, comprising:
(a) a first single chain polypeptide comprising a binding domain that specifically binds a target, a hinge, a first immunoglobulin heterodimerization domain, and an Fc region portion; and
(b) a second single chain polypeptide comprising a hinge, a second immunoglobulin heterodimerization domain that is not the same as the first immunoglobulin heterodimerization domain of the first single chain polypeptide, and an Fc region portion;
wherein the first and second immunoglobulin heterodimerization domains associate with each other to form a polypeptide heterodimer comprised of the first and the second single chain polypeptides, and wherein
(i) the first immunoglobulin heterodimerization domain comprises a first immunoglobulin CH1 region and the second immunoglobulin heterodimerization domain comprises a first immunoglobulin CL region, or,
(ii) the first immunoglobulin heterodimerization domain comprises a first immunoglobulin CL region and the second immunoglobulin heterodimerization domain comprises a first immunoglobulin CH1 region, and
wherein the Fc region portion comprises an immunoglobulin CH2 domain of IgG1, IgG2, IgG3, IgG4, IgA1, IgA2, IgD, or any combination thereof; an immunoglobulin CH2 domain and CH3 domain of IgG1, IgG2, IgG3, IgG4, IgA1, IgA2, IgD, or any combination thereof; an immunoglobulin CH3 domain of IgG1, IgG2, IgG3, IgG4, IgA1, IgA2, IgD, IgE, IgM, or any combination thereof; or an immunoglobulin CH3CH4 domain of IgE, IgM, or a combination thereof.

2. The polypeptide heterodimer of claim 1, wherein the binding domain is a single chain Fv (scFv).

3. The polypeptide heterodimer of claim 1, wherein the binding domain is amino terminal to the Fe region portion.

4. The polypeptide heterodimer of claim 1, wherein the binding domain is carboxyl terminal to the Fe region portion.

5. The polypeptide heterodimer of claim 1, wherein the binding domain specifically binds to c-Met, RON, CD3, CEACAM6, EGFR, ErbB3, ErbB4, EphA2, GITR, IGFIR, GHRHR, GHR, FLT1, KDR, FLT4, CD44v6, CD151, TGFBR2, TGFBR1, IL6R, gp130, TNFR1, TNFR2, PD1, PD-L1, PD-L2, BTLA, HVEM, RANK, TNFRSF4, CD40, CD137, TWEAK-R, LTfβR, LIFRβ, OSMRβ, TCRα, TCRβ, CD19, CD28, CD80, CD81, CD86, TLR7, TLR9, PTCH1, LRP5, Frizzled-1, or Robo1.

6. The polypeptide heterodimer of claim 1, wherein the first immunoglobulin heterodimerization domain comprises the first immunoglobulin CH1 region and the second immunoglobulin heterodimerization domain comprises the first immunoglobulin CL region.

7. (canceled)

8. (canceled)

9. The polypeptide heterodimer of claim 6 wherein the first single chain polypeptide further comprises a second CH1 region and the second single chair polypeptide further comprises a second CL region, and wherein the second CH1 region of the first single chain polypeptide and the second CL region of the second single chain polypeptide associate with each other in the polypeptide heterodimer.

10-12. (canceled)

13. The polypeptide heterodimer of claim 1, wherein the first immunoglobulin heterodimerization domain comprises a first immunoglobulin CL region and the second immunoglobulin heterodimerization domain comprises a first immunoglobulin CH1 region.

14. (canceled)

15. (canceled)

16. The polypeptide heterodimer of claim 13 wherein the first single chain polypeptide further comprises a second CL region and the second single chain polypeptide further comprises a second CH1 region, and wherein the second CL region of the first single chain polypeptide and the second CH1 region of the second single chain polypeptide associate with each other in the polypeptide heterodimer.

17-19. (canceled)

20. The polypeptide heterodimer of claim 6 wherein the first single chain polypeptide further comprises a second CL region and the second single chain polypeptide further comprises a second CH1 region, and wherein the second CL region of the first single chain polypeptide and the second CH1 region of the second single chain polypeptide associate with each other in the polypeptide heterodimer.

21-26. (canceled)

27. The polypeptide heterodimer of claim 1, wherein the e first CL region is a 78 region or a Cλ region.

28-30. (canceled)

31. The polypeptide heterodimer of claim 27, wherein the Cκ region is a wild type human immunoglobulin 78 region.

32. The polypeptide heterodimer of claim 27, wherein the Cκ region is an altered human immunoglobulin Cκ region in which one or more amino acids of a wild type human Cκ region are substituted at N29, N30, Q52, V55, T56, S68, or T70.

33. (canceled)

34. The polypeptide heterodimer of claim 27, wherein the CH1 region is an altered human immunoglobulin CH1 region comprising an amino acid substitution by which Val (V) at position 68 is substituted by Lys (K), Arg (R) or His (H), and wherein the Cκ region is an altered human immunoglobulin Cκ region comprising an amino acid substitution by which Leu (L) at position 27 is substituted by Asp (D) or Glu (E).

35. The polypeptide heterodimer of claim 27, wherein the CH1 region is an altered human immunoglobulin CH1 region comprising an amino acid substitution by which Val (V) at position 68 is changed to Asp (D) or Glu (F), and wherein the Cκ region is an altered human immunoglobulin Cκ region comprising an amino acid substitution by which Leu (L) at position 2729 is changed to Lys (K), Arg (R) or His (H).

36. (canceled)

37. (canceled)

38. The polypeptide heterodimer of claim 1, wherein the first CH 1 region an altered human immunoglobulin CH1 region with the cysteine of a wild type human immunoglobulin CH1 region that is involved in forming a disulfide bond with a wild type human immunoglobulin CL region is deleted or substituted.

39. (canceled)

40. (canceled)

41. The polypeptide heterodimer of claim 38, wherein the first CH1 region is a polypeptide comprising SEQ ID NO: 114.

42. The polypeptide heterodimer of claim 27, wherein the Cκ region is selected from polypeptides comprising SEQ ID NOS:141-178 and 202.

43. The polypeptide heterodimer of claim 27, wherein the Cλregion is a polypeptide comprising SEQ ID NO: 140.

44. The polypeptide heterodimer of claim 1, wherein the Fc region portion comprises an immunoglobulin CH2 domain.

45-49. (canceled)

50. The polypeptide heterodimer of claim 1, wherein the Fc region portion comprises an immunoglobulin CH12 domain and an immunoglobulin CH3 domain.

51-57. (canceled)

58. The polypeptide heterodimer of claim 1, wherein the hinge of both the first and second single chain polypeptides is an immunoglobulin hinge region.

59-61. (canceled)

62. The polypeptide heterodimer of claim 58, wherein the immunoglobulin hinge region is
(a) present amino terminal to the Fe region portion,
disposed between the binding domain and the immunoglobulin heterodimerization domain.
(c) disposed between the immunoglobulin heterodimerization domain and the Fc region portion, or
(d) at the amino terminus of the first or second single chain polypeptide.

63-67. (canceled)

68. The polypeptide heterodimer of claim 1, wherein the hinges of the first and second single chain polypeptides are different.

69. The heterodimer of claim 1, wherein the first single chain polypeptide comprises amino acids 21-609 of SEQ ID NO:26, and the second single chain polypeptide comprises amino acids 21-363 of SEQ ID NO: 137; the first single chain polypeptide comprises amino acids 21-716 of SEQ ID NO:46, and the second single chain polypeptide comprises amino acids 21-461 of SEQ ID NO:48; the first single chain polypeptide comprises amino acids 21-716 of SEQ ID NO:46 and the second single chain polypeptide comprises amino acids 21-461 of SEQ ID NO:64, the first single chain polypeptide comprises amino acids 21-716 of SEQ ID NO:62, and the second single chain polypeptide comprises amino acids 21-461 of SEQ ID NO:48; or the first single chain polypeptide comprises amino acids 21-716 of SEQ ID NO:62, and the second single chain polypeptide comprises amino acids 21-461 of SEQ ID NO:64; the first single chain polypeptide comprises SEQ ID NO: 139, and the second single chain polypeptide comprises amino acids of 21-461 of SEQ ID NO:48; the first single chain polypeptide comprises SEQ ID NO: 263, and the second single chain polypeptide comprises amino acids of 21-461 of SEQ ID NO:48; the first single chain polypeptide comprises SEQ ID NO:267, and the second single chain polypeptide comprises amino acids of 21-461 of SEQ ID NO:48, the first single chain polypeptide comprises SEQ ID NO:769, and the second single chain polypeptide comprises SEQ ID NO:765; the first single chain polypeptide comprises SEQ ID NO:769, and the second single chain polypeptide comprises SEQ ID NO:766; the first single chain polypeptide comprises SEQ ID NO:769, and the second single chain polypeptide comprises SEQ ID NO:767; the first single chain polypeptide comprises SEQ ID NO:769, and the second single chain polypeptide comprises SEQ ID NO:768; the first single chain polypeptide comprises SEQ ID NO:781, and the second single chain polypeptide comprises SEQ ID NO:778; and the first single chain polypeptide comprises SEQ ID NO:780; and the second single chain polypeptide comprises SEQ ID NO: 779.

70. The heterodimer of claim 1, wherein the first single chain polypeptide comprises amino acids 21-609 of SEQ ID NO:22 or 21-595 of SEQ ID NO: 135, and the second single chain polypeptide comprises SEQ ID NO:91, 92, 193. 98, 99, 101, or 103, or amino acids 21-361 of SEQ ID NO: 129, 131 or 133.

71. (canceled)

72. A composition comprising a polypeptide heterodimer of claim 1 and a pharmaceutically acceptable excipient.

73. An expression vector capable of expressing the polypeptide heterodimer of claim 1, comprising a first polynucleotide encoding the first single chain polypeptide and a second polynucleotide encoding the second single chain polypeptide.

74. A host cell comprising the expression vector of claim 73.

75. A host cell comprising first and second expression vectors capable of expressing the first and second single chain polypeptides, respectively, of the polypeptide heterodimer of claim 1.

76. A method for making a polypeptide heterodimer, comprising
(a) culturing a host cell of claim 74 under conditions suitable to express first and second single chain polypeptides, and
(b) optionally isolating or purifying the heterodimers formed from the first and second single chain polypeptides from the culture.

77. A method for reducing T cell activation, comprising administering to a patient in need thereof an effective amount of a heterodimer of claim 1, wherein the binding domain specifically binds CD28.

78. A method for inhibiting growth of a solid malignancy, comprising administering to a patient in need thereof an effective amount of a heterodimer of claim 1, wherein the binding domain specifically binds EGFR, ErbB3, ErbB4, c-Met, RON, CEACAM6, EphA2, IGF1R, GHRHR, GHR, VEGFR1, VEGFR2, VEGFR3, CD44v6, CD151, TGFBR2, IL6R, gp130, TNFR2, PD1, TWEAK-R, OSMRbeta, Patched-1, Frizzled, or Robo1.

79. A method for treating an autoimmune or inflammatory condition, comprising administering to a patient in need thereof an effective amount of a heterodimer according to claim 1, wherein the binding domain specifically binds TGFBR2, TGFBR1, IL6R, gp130, TNFR1, TNFR2, PD1, HVEM, OX40, CD40, CD137, TWEAK-R, LTβR, LIFRβ, OSMRβ, CD3, TCRα, TCRβ, CD19, CD28, CD80, CD81, CD86, TLR7, or TLR9.

80-83. (canceled)
